SEQUENCE LISTING (52 sequences)

SEQ ID NO: 1 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 2 - protein, 189 aa, artificial
  mutant of H-Ras
MTEYKLVVVGAGGVGKSAATIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - protein, 189 aa, artificial
  mutant of H-Ras
MTEYKLVVVGAGGVGKSAGTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 4 - protein, 189 aa, artificial
  mutant of H-Ras
MTEYKLVVVGAGGVGKSALTIQLIQNHAVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 5 - protein, 189 aa, artificial
  mutant of H-Ras
MTEYKLVVVGAGGVGKSALTIQLIQNHGVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 6 - protein, 189 aa, artificial
  mutant of H-Ras
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGAKCDLAARTVESRQAQDLARSYGIPYIETSAKTRQGVEDAFYTLVREIRQHKLRKLNPPDESGPGCMSCKCVLS

SEQ ID NO: 7 - protein, 189 aa, artificial
  mutant of H-Ras
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGGKCDLAARTVESRQAQDLARSYGIPYIETSAKTRQGVEDAFYTLVREIRQHKLRKLNPPDESGPGCMSCKCVLS

SEQ ID NO: 8 - protein, 189 aa, artificial
  mutant of H-Ras
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNACDLAARTVESRQAQDLARSYGIPYIETSAKTRQGVEDAFYTLVREIRQHKLRKLNPPDESGPGCMSCKCVLS

SEQ ID NO: 9 - protein, 189 aa, artificial
  mutant of H-Ras
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNGCDLAARTVESRQAQDLARSYGIPYIETSAKTRQGVEDAFYTLVREIRQHKLRKLNPPDESGPGCMSCKCVLS

SEQ ID NO: 10 - protein, 189 aa, artificial
  mutant of H-Ras
MTEYKLVVVGAGGVGKSAATIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGAKCDLAARTVESRQAQDLARSYGIPYIETSAKTRQGVEDAFYTLVREIRQHKLRKLNPPDESGPGCMSCKCVLS

SEQ ID NO: 11 - protein, 189 aa, artificial
  mutant of H-Ras
MTEYKLVVVGAGGVGKSAATIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGGKCDLAARTVESRQAQDLARSYGIPYIETSAKTRQGVEDAFYTLVREIRQHKLRKLNPPDESGPGCMSCKCVLS

SEQ ID NO: 12 - protein, 189 aa, artificial
  mutant of H-Ras
MTEYKLVVVGAGGVGKSAGTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGAKCDLAARTVESRQAQDLARSYGIPYIETSAKTRQGVEDAFYTLVREIRQHKLRKLNPPDESGPGCMSCKCVLS

SEQ ID NO: 13 - protein, 189 aa, artificial
  mutant of H-Ras
MTEYKLVVVGAGGVGKSAGTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGGKCDLAARTVESRQAQDLARSYGIPYIETSAKTRQGVEDAFYTLVREIRQHKLRKLNPPDESGPGCMSCKCVLS

SEQ ID NO: 14 - protein, 189 aa, artificial
  mutant of H-Ras
MTEYKLVVVGAGGVGKSACTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGAKCDLAARTVESRQAQDLARSYGIPYIETSAKTRQGVEDAFYTLVREIRQHKLRKLNPPDESGPGCMSCKCVLS

SEQ ID NO: 15 - protein, 189 aa, artificial
  mutant of H-Ras
MTEYKLVVVGAGGVGKSACTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGGKCDLAARTVESRQAQDLARSYGIPYIETSAKTRQGVEDAFYTLVREIRQHKLRKLNPPDESGPGCMSCKCVLS

SEQ ID NO: 16 - protein, 189 aa, artificial
  mutant of H-Ras
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGCKCDLAARTVESRQAQDLARSYGIPYIETSAKTRQGVEDAFYTLVREIRQHKLRKLNPPDESGPGCMSCKCVLS

SEQ ID NO: 17 - protein, 189 aa, artificial
  mutant of H-Ras
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGGKCDLAARTVESRQAQDLARSYGIPYIECSAKTRQGVEDAFYTLVREIRQHKLRKLNPPDESGPGCMSCKCVLS

SEQ ID NO: 18 - DNA, 570 bp, Homo sapiens
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 19 - DNA, 570 bp, artificial
  mutant of H-Ras
atgacggaatataagctggtggtggtgggcgccggcggtgtgggcaagagtgcggcgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 20 - DNA, 570 bp, artificial
  mutant of H-Ras
atgacggaatataagctggtggtggtgggcgccggcggtgtgggcaagagtgcggggaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 21 - DNA, 570 bp, artificial
  mutant of H-Ras
atgacggaatataagctggtggtggtgggcgccggcggtgtgggcaagagtgcgctgaccatccagctgatccagaaccatgctgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 22 - DNA, 570 bp, artificial
  mutant of H-Ras
atgacggaatataagctggtggtggtgggcgccggcggtgtgggcaagagtgcgctgaccatccagctgatccagaaccatggtgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 23 - DNA, 570 bp, artificial
  mutant of H-Ras
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtgggggccaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 24 - DNA, 504 bp, artificial
  mutant of H-Ras
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggggcaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctg

SEQ ID NO: 25 - DNA, 504 bp, artificial
  mutant of H-Ras
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacgcgtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctg

SEQ ID NO: 26 - DNA, 504 bp, artificial
  mutant of H-Ras
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacgggtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctg

SEQ ID NO: 27 - DNA, 570 bp, artificial
  mutant of H-Ras
atgacggaatataagctggtggtggtgggcgccggcggtgtgggcaagagtgcggcgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtgggggccaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 28 - DNA, 570 bp, artificial
  mutant of H-Ras
atgacggaatataagctggtggtggtgggcgccggcggtgtgggcaagagtgcggcgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggggcaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 29 - DNA, 570 bp, artificial
  mutant of H-Ras
atgacggaatataagctggtggtggtgggcgccggcggtgtgggcaagagtgcggggaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtgggggccaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 30 - DNA, 570 bp, artificial
  mutant of H-Ras
atgacggaatataagctggtggtggtgggcgccggcggtgtgggcaagagtgcggggaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggggcaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 31 - DNA, 570 bp, artificial
  mutant of H-Ras
atgacggaatataagctggtggtggtgggcgccggcggtgtgggcaagagtgcgtgtaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtgggggccaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 32 - DNA, 570 bp, artificial
  mutant of H-Ras
atgacggaatataagctggtggtggtgggcgccggcggtgtgggcaagagtgcgtgtaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggggcaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 33 - DNA, 570 bp, artificial
  mutant of H-Ras
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggtgcaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 34 - DNA, 570 bp, artificial
  mutant of H-Ras
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggggcaagtgtgacctggctgcacgcactgtggaatctcggcaggctcaggacctcgcccgaagctacggcatcccctacatcgagtgctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 35 - protein, 12 aa, Homo sapiens
GAGGVGKSALTI

SEQ ID NO: 36 - protein, 7 aa, Homo sapiens
LVGNKCD

SEQ ID NO: 37 - protein, 12 aa, Unknown
  unknown species
GDSGVGKTSLMN

SEQ ID NO: 38 - protein, 7 aa, Unknown
  unknown species
VLGNKID

SEQ ID NO: 39 - protein, 12 aa, Unknown
  unknown species
GDGGTGKTTFVK

SEQ ID NO: 40 - protein, 7 aa, Unknown
  unknown species
LCGNKVD

SEQ ID NO: 41 - protein, 12 aa, Unknown
  unknown species
GSGGVGKSALTL

SEQ ID NO: 42 - protein, 7 aa, Unknown
  unknown species
VVGNKSD

SEQ ID NO: 43 - protein, 12 aa, Unknown
  unknown species
GSGGVGKSALTV

SEQ ID NO: 44 - protein, 7 aa, Unknown
  unknown species
LVGNKCD

SEQ ID NO: 45 - protein, 12 aa, Unknown
  unknown species
GDGACGKTCLLI

SEQ ID NO: 46 - protein, 7 aa, Unknown
  unknown species
LVGNKKD

SEQ ID NO: 47 - protein, 12 aa, Unknown
  unknown species
GLDCAGKTTVLY

SEQ ID NO: 48 - protein, 7 aa, Unknown
  unknown species
IVANKQD

SEQ ID NO: 49 - protein, 12 aa, Unknown
  unknown species
GAGESGKSTIVK

SEQ ID NO: 50 - protein, 7 aa, Unknown
  Unknown species
LFLNKKD

SEQ ID NO: 51 - protein, 12 aa, Unknown
  unknown species
GHVDSGKSTTTG

SEQ ID NO: 52 - protein, 7 aa, Unknown
  unknown species
VGVNKMD

(19) United States
(12) Patent Application Publication
(10) Pub. No.: US 2004/0241706 A1
(43) Pub. Date: Dec. 2, 2004

(54) Highly specific modulators of GTPases for target validation

(75) Inventors: Kavita Shah, San Diego, CA (US); Fabien Vincent, San Diego, CA (US); Maria A. Cueto, Weehawken, NJ (US)

Correspondence Address: QUINE INTELLECTUAL PROPERTY LAW GROUP, P.C., P O BOX 458, ALAMEDA, CA, 94501, US

(73) Assignees: IRM, LLC; Novartis Pharmaceuticals Corporation

(21) Appl. No.: 10/660,113

(22) Filed: Sep. 10, 2003

Related U.S. Application Data
(60) Provisional application No. 60/410,536, filed on Sep. 13, 2002.
(60) Provisional application No. 60/461,755, filed on Apr. 9, 2003.

(51) Int. Cl. C12Q 1/68; C07H 21/04; A61K 48/00

(52) U.S. Cl. 435/6; 435/69.1; 435/375; 514/44; 536/23.2

(57) ABSTRACT

This invention provides methods and compositions related to modified GTPase sequences and GTPase modulators.

### CROSS REFERENCE TO RELATED APPLICATION

This application is related to U.S. provisional patent applications U.S. Ser. No. 60/410,536 filed Sep. 13, 2002 and U.S. Ser. No. 60/461,755 filed Apr. 9, 2003. The present application claims priority to, and benefit of, these applications, pursuant to 35 U.S. C. § 119(e) and any other applicable statute or rule.

### FIELD OF THE INVENTION

The present invention is in the fields of biomolecular engineering and enzyme activity modulation.

### BACKGROUND OF THE INVENTION

The human genome contains more than 160 GTPases (Venter et al. (2001). “The sequence of the human genome” Science 291:1304-51) encompassing Ras family members, translation elongation factors and the alpha subunits of heterotrimeric G proteins. Most cellular processes such as protein biosynthesis, translocation processes in the endoplasmic reticulum, vesicle trafficking, control of cell differentiation, proliferation and oncogenesis, T-cell activation, and transmembrane signal transduction are known to depend on GTPases. With the exception of elongation factors, they act as precisely engineered molecular switches and are deeply woven in signal transduction networks (Vetter & Wittinghofer (2001) “The guanine nucleotide-binding switch in three dimensions” Science 294: 1299-304). Binding of GTP or GDP locks these proteins into dissimilar “on” and “off” conformations, respectively; and thereby regulates their affinities to other macromolecules (effectors) which transmit the signal downstream (FIG. 32). A wide variety of diseases take their roots in deregulated GTPase activities or have harmful signals conveyed through them (Benard et al. (1999) “Potential drug targets: small GTPases that regulate leukocyte function” Trends Pharmacol. Sci. 20:365-70; Boquet et al. (1999) “Rho GTP-binding proteins as targets for microbial pathogens” Prog. Mol. Subcell. Biol. 22:183-99; de Gunzburg (1999) “Proteins of the Ras pathway as novel potential anticancer therapeutic targets” Cell. Biol. Toxicol. 15:345-58). Notably, their susceptibility to microbial toxins and mutations has implicated them in infectious diseases and tumorigenesis. For example, deregulated Ras activity is present in as much as 60% of all cancers and is considered a driving force of cellular proliferation (de Gunzburg (1999) “Proteins of the Ras pathway as novel potential anticancer therapeutic targets” Cell Biol. Toxicol. 15:345-58). Despite their great potential as drug targets, no active site-directed inhibitors are known to date. Traditionally, the surprising affinity of GTPases for their substrates (Kₐ˜10 pM) and the high intracellular concentration of GTP (˜1 mM) have been blamed for this failure (Downward (2003) “Targeting Ras signaling pathways in cancer therapy” Nat. Rev. Cancer 3:11-22). Equally important, this lack of specific and temporal control over the activities of GTPases has impeded the elucidation of their pathways. This is in sharp contrast with another family of signal transduction proteins, kinases, where the availability of even partly selective inhibitors greatly facilitated their functional study in cellular systems.

The convergent engineering of small molecule/protein interfaces to address biological questions has emerged as a powerful new tool (Schreiber (1998) “Chemical genetics resulting from a passion for synthetic organic chemistry” Bioorg. Med. Chem. 6: 1127-52; Bishop et al. (2000) “Unnatural ligands for engineered proteins: new tools for chemical genetics” Annu. Rev. Biophys. Biomol. Struct. 29, 577-606). This strategy allows tight control over biological activities by combining the advantages of approaches based on chemistry (speed of action, e.g., of small molecules) and genetics (absolute specificity). Diverse systems have been studied using this methodology including 7-transmembrane receptors (Coward et al. (1998) “Controlling signaling with a specifically designed Gi-coupled receptor” Proc Natl Acad Sci U S A 95: 352-7), nuclear hormone receptors (Peet et al. (1998) “Engineering novel specificities for ligand-activated transcription in the nuclear hormone receptor RXR” Chem Biol 5: 13-21), G proteins (Hwang & Miller (1987) “A mutation that alters the nucleotide specificity of elongation factor Tu, a GTP regulatory protein” J Biol Chem 262: 13081-5, kinases (Shah et al. (1997) “Engineering unnatural nucleotide specificity for Rous sarcoma virus tyrosine kinase to uniquely label its direct substrates” Proc Natl Acad Sci U S A 94: 3565-70) and methyltransferases (Lin et al. (2001) “Design of allele-specific protein methyltransferase inhibitors” J Am Chem Soc 123: 11608-13). Additionally, chemical inducers of dimerization (CIDs) were used successfully to unravel various signaling pathways (Spencer et al. (1993) “Controlling signal transduction with synthetic ligands” Science 262: 1019-24). In particular, we and others have applied the chemical-genetic strategy to the study of kinases, by designing substrates and inhibitors specific for the mutated enzymes (Shah et al. (1997) “Engineering unnatural nucleotide specificity for Rous sarcoma virus tyrosine kinase to uniquely label its direct substrates” Proc Natl Acad Sci U S A 94: 3565-70; Bishop et al. (2000) “A chemical switch for inhibitor-sensitive alleles of any protein kinase”, Nature 407: 395-401; Shah & Shokat (2002) “A chemical genetic screen for direct v-Src substrates reveals ordered assembly of a retrograde signaling pathway” Chem Biol 9: 35-47; Holt et al. (2002) “A chemical-genetic strategy implicates myosin-1c in adaptation by hair cells” Cell 108: 371-81). Functional assignment of kinases can then be achieved through identification of novel protein substrates and DNA microarray studies. Importantly, the conserved character of the ATP binding pocket in kinases means that translation of this method to other kinases is easily achieved Bishop et al. (2000) “A chemical switch for inhibitor-sensitive alleles of any protein kinase” Nature 407: 395-401.

GTPases themselves were the object of pioneering chemical-genetic studies Hwang & Miller (1987) “A mutation that alters the nucleotide specificity of elongation factor Tu, a GTP regulatory protein” J Biol Chem 262: 13081-5. A change in substrate specificity from GTP to XTP (xanthosine 5′-triphosphate) was achieved by mutating a conserved aspartate residue (D119 in H-Ras) interacting with the exocyclic C(2) amine of GTP to asparagine. The protein-nucleotide hydrogen bond thus abolished can be reestablished if XTP is used rather than GTP. This system was transposed to multiple GTPases and enabled their study using in vitro systems (Weijland et al. (1994) “Elongation factor Tu D138N, a mutant with modified substrate specificity, as a tool to study energy consumption in protein biosynthesis” Biochemistry 33: 10711-7; Bishop et al. (2000) “Unnatural ligands for engineered proteins: new tools for chemical genetics” Annu Rev Biophys Biomol Struct 29: 577-606). However, the intracellular levels of xanthosine, XDP and XTP are tightly regulated and appear difficult to manipulate. Since the mutant enzymes do not bind to their natural substrates GDP and GTP properly, this limitation rendered the application of this approach to in vivo studies almost impracticable (Cool et al. (1999) “The Ras mutant D119N is both dominant negative and activated” Mol Cell Biol 19: 6297-305; Shokat & Velleca (2002) “Novel chemical genetic approaches to the discovery of signal transduction inhibitors” Drug Discov Today 7: 872-9). As a result, the design of a mutant GTPase retaining its natural substrate selectivity (GDP and GTP binding) but susceptible to a uniquely specific inhibitor or activator is highly desirable. Such a system would greatly facilitate the functional study of a GTPase of interest by allowing its activity to be specifically and temporally controlled. In addition, identification of proteins capable of binding to a specific GTPase, either in the GTP- or GDP-bound form, is of significant interest in elucidating the functional pathways in which GTPases act.

The present invention addresses this need in the art by providing engineered GTPases and GTPase activity modulators that can be used together to analyze and/or modulate GTPase activity and biological pathways, and to identify upstream and downstream effectors of GTPase function. In addition, the mutant GTPases and activity modulators of the present invention satisfy a need in the art for compositions useful in the diagnosis, treatment and prevention of GTPase-involved diseases and disorders.

### SUMMARY OF THE INVENTION

A “chemical genetic” approach to analysis of protein function and design of activity modulators (“modulators”), e.g., inhibitors and activators, was taken towards the generation of novel modulators for GTPase enzymatic systems (for an overview of “chemical genetic” approaches to protein design, see, for example, Bishop et al. (2000) Annual Rev. Biophys. Biomol. Struct. 29:577-606). The GTPase enzymes were engineered to accept orthogonal activity modulators (i.e., compounds modified to no longer interact with the original target molecule, in this case, the corresponding wild-type GTPase molecule). The changes in chemical structure of the modulator alter, and potentially provide additional, molecular interactions (as compared to the GTP and GDP ligands). In addition, the mutations in protein sequence serve to modify the substrate recognition and/or binding characteristics, possibly decreasing the affinity of the mutant enzyme for the natural substrate (e.g., GTP or GDP). Optionally, the changes in protein structure also create a large enough “hole” in or near the GTP binding site to accommodate bulkier modulator structures, which have a greater likelihood of not interacting with the wild-type protein.

Accordingly, the present invention provides mutant GTPases having a non-native amino acid at one, two, or more amino acid positions that correspond to, for example, N116, T144 and L19 of the H-Ras sequence. The mutant GTPases are capable of binding to one or more orthogonal GTPase modulators, e.g., compounds that inhibit or activate the mutant GTPase but do not substantially inhibit or activate a corresponding wild-type GTPase. Optionally, the mutant GTPase is a mutant Ras GTPase.

In one embodiment, the non-native amino acid(s) provided by the mutation is alanine, glycine, or cysteine (e.g., one or more substitutions corresponding to L19A, L19C, L19G, N116A, N116C, N116G, and T144C are present in the mutant protein). In a preferred embodiment, the mutant GTPase includes non-native amino acids at both positions L19 and N116. Optionally, the presence of the non-native amino acid cysteine can be used to form a covalent bond (for example, a disulfide linkage) with an appropriate substituent (e.g., a sulfhydryl group or an electrophilic moiety) of the GTPase modulator. Exemplary amino acid sequences embodying the mutant GTPases of the present invention are provided by SEQ ID No. 2 to 17; corresponding nucleic acid sequences encoding the exemplary GTPases are provided by SEQ ID No. 19 to 34.

The GTPase activity modulators of the present invention include, but are not limited to, chemical structures selected from the group consisting of a) a guanine ring modified at one or more of a C-6, N-7 and/or N-9 position of the purine ring; b) a guanosine ring modified at the C-2, C-6 and/or N-7 of the purine ring; or c) a phosphorylated guanosine composition (e.g., a GMP, GDP or GTP structure) modified at a C-6 and/or N-7 position of the purine ring. In the modulator embodiments involving substitution at the N-7 position of the purine ring, the nitrogen atom is optionally replaced by a carbon atom. The present invention also provides methods of synthesizing the GTPase modulators. In some embodiments, the GTPase modulator includes an electrophilic moiety, sulfhydryl group, or other moiety capable of forming a covalent bond with an element of the mutant GTPase molecule. Optionally, the GTPase modulator is a cell permeable compound.

In one embodiment, the GTPase modulators of the present invention comprise a substituted guanine ring having a) an O-propyl group, an O-isopropyl group, an O-isobutyl group, an O-sec-butyl group, an O-methyl-t-butyl group, an O-(2,2-dimethyl)propyl group, an O-cyclohexyl group, an O-methyl-cyclohexyl group, an O-(2-cyclohexyl)ethyl group, an O-(3-cyclohexyl)propyl group, an O-benzyl group, an O-(2-phenyl)ethyl group, an O-[2-(1-naphthyl)ethyl group, an O-[2-(2-naphthyl)]ethyl group, or the corresponding N-linked structures at the C-6 position of the purine ring; and b) a benzyl or a (2,2-dimethyl) propyl substituent at the N-9 position.

In another embodiment, the GTPase modulators of the present invention comprise a substituted mono-phosphorylated, di-phosphorylated, or tri-phosphorylated guanosine nucleotide. The substituted nucleotide includes substitutions at C-6, N-7, or both positions of the purine ring; the phosphorylated ribose moiety is coupled at the N-9 position. Optionally, the phosphate groups can be “caged,” e.g. covalently bound to substituents that can be released at a selected moment, for example, after entry of the compound into the cytosol of a cell.

In a further embodiment, the GTPase modulators of the present invention comprise a substituted guanosine nucleoside structure (e.g., the guanine ring and ribose constituents, without any phosphate groups) having substituents at the C-6, N-7, or both C-6 and N-7 positions of the purine ring.

In a further aspect, the present invention also provides complexes comprising a mutant GTPase of the present invention bound to a GTPase modulator that inhibits the mutant GTPase but does not substantially inhibit a corresponding wild-type GTPase (i.e., an orthogonal inhibitor). Alternatively, the present invention provides complexes comprising a mutant GTPase bound to a GTPase modulator that activates the mutant GTPase but does not substantially activate a corresponding wild-type GTPase (i.e., an orthogonal activator). In one embodiment, the GTPase-modulator complex is covalently coupled.

In addition, the present invention provides host cells comprising a mutant GTPase, which mutant GTPase comprises a non-native amino acid at one or more amino acid positions that correspond to N116, T144 and L19 of Ras, which mutant GTPase binds to a GTPase activity modulator that inhibits the mutant GTPase but does not substantially inhibit a corresponding wild-type GTPase, or that activates the mutant GTPase but does not substantially activate a corresponding wild-type GTPase. Optionally, the host cell is a plant or animal cell; preferably, the host cell is a mammal cell. Exemplary cells include, but are not limited to, fibroblasts, epithelial cells, endothelial cells, myeloid leukemia cells, Raji cells, pancreatic cells, human glioma/glioblastoma cell lines, and the like. Optionally, the host cells of the present invention do not express the wild-type GTPase that corresponds to the mutant GTPase. For example, the gene(s) in the host cell that encodes the wild-type GTPase can be disrupted, such that the host cell does not synthesize the protein. One mechanism of achieving such disruption is by contacting the host cell with an antisense nucleic acid or a small interfering RNA (siRNA) that inhibits expression of the corresponding wild-type GTPase but not the mutant GTPase.

The present invention also provides methods of determining a GTPase function. The methods include the steps of a) expressing at least one mutant GTPase in one or more host cells; b) contacting the mutant GTPase with at least one GTPase modulator that binds to the mutant GTPase but does not substantially modulate, i.e., inhibit or activate, a corresponding wild-type GTPase; and c) detecting at least one result of applying the GTPase modulator to the cell, thereby determining the function of the GTPase.

In another embodiment, the present invention provides methods of determining one or more GTPase binding proteins, or effector molecules, which interact with a selected GTPase protein. The methods include the steps of a) providing a mutant GTPase, which retains the effector specificity (binding properties) of a corresponding wild-type GTPase; b) contacting the mutant GTPase with at least one GTPase modulator that binds to the mutant GTPase but does not substantially inhibit or activate the corresponding wild-type GTPase; c) contacting the mutant GTPase complex including the orthogonal modulator with at least one GTPase binding protein or effector molecule; and d) detecting the GTPase effector molecule, thereby determining one or more effector molecules that bind the GTPase. Detection can be performed, for example, by isolation and sequencing of a bound effector molecule. In an embodiment, the method involves expressing the mutant GTPase, e.g., a mutant GTPase comprising a sequence tag, in vivo, and purifying the mutant GTPase on a solid substrate. Following contact and binding with an effector molecule, the GTPase can be eluted and the effector detected by, e.g., two dimensional gel electrophoresis, mass spectrometry, and the like. See, for example, PCT publication WO 03/054772 to Brock et al.

Typically, the mutant GTPase employed in the methods of the present invention has a decreased affinity for GTP and/or GDP as compared to the wild-type enzyme. Optionally, the mutant GTPase is a modified form of a Ras-type GTPase. In some embodiments of the methods, the mutant GTPase employed in the method includes a cysteine residue (at, for example, amino acid position L19, N116, or T144), and the GTPase modulator comprises a sulfhydryl group. In an alternate embodiment, the GTPase modulator includes an affinity label, such as an electrophilic moiety. In these embodiments, contact between the mutant GTPase and modulator optionally leads to the formation of a covalent linkage between the two moieties, altering the GTPase activity. One useful class of affinity labels are cysteine affinity labels, including, but not limited to, reagents containing a maleimide, alkyl halide or acrylamide moiety. Reaction of the affinity label with a cysteine residue in the GTPase protein sequence can lead to formation of a covalent linkage, potentially reducing or altering the GTPase activity.

In some embodiments, the methods employ a guanosine-containing moiety (e.g., guanosine, GMP, GDP or GTP) having substituents at one or more of the C-6 and N-7 positions as the GTPase modulator. Optionally, the GTPase modulator is capable of competing with GTP and/or GDP for the active site of the mutant GTPase.

Detecting the result of applying the GTPase modulator to the cell includes, but is not limited to, performing one or more assays to detect one or more functions of the mutant GTPase (e.g., binding of regulatory or effector molecules, binding or release of GTP, GDP, or phosphate), or examining one or more downstream response pathways affected by modulating the GTPase. Optionally, the method further includes collecting data regarding the downstream response pathways and storing the data in at least one database. In some embodiments, a gene expression profile of the cell is generated both in the presence and absence of the GTPase modulator, thereby identifying genes that are upregulated or downregulated in the presence of the GTPase modulator.

In addition, the present invention also provides methods of reducing activity of a GTPase in a cell. The methods include, but are not limited to, the steps of a) introducing a mutant GTPase into the cell, wherein the mutant GTPase comprises a non-native amino acid at one or more amino acid positions that correspond to N116, T144 and L19 of H-Ras, which mutant GTPase binds to a GTPase modulator that alters the activity of the mutant GTPase but does not substantially alter the activity, e.g., inhibit or activate a corresponding wild-type GTPase; and b) contacting the mutant GTPase with the GTPase modulator, thereby competing with the wild-type GTPase for binding to one or more cellular effector molecules and reducing the activity of the GTPase in the cell. Optionally, the cell is present in an animal, and the GTPase modulator comprises a cell permeable compound. In one embodiment, the method further comprises the step of administering an antisense nucleic acid or an siRNA that inhibits expression of the corresponding wild-type GTPase but not the mutant GTPase, thereby reducing (or further reducing) the GTPase activity in the cell.

Thus, the present invention provides mutant (engineered) GTPases and GTPase modulators that can be used together to analyze and/or modulate GTPase activity. The compositions and methods of the present invention can be used to investigate the roles that specific GTPases perform, an examination previously unrealized due to the lack of suitable specific small molecule modulators. In addition, the mutant GTPases and modulators of the present invention are also useful in the diagnosis, treatment and prevention of GTPase-involved diseases and disorders.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a modulator” includes combinations of modulators; reference to “a GTPase” includes families of GTPases, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

The term “GTPase” as used herein refers to a guanosine triphosphatase, e.g., an enzyme (or portion thereof) capable of binding GTP and GDP and/or hydrolyzing GTP to GDP. A GTPase protein is capable of binding a “substrate” molecule (e.g., GTP, GDP, modulator, inhibitor, activator, analogue) as well as an “effector” molecule (adenylate cyclase, etc).

A “non-native” amino acid is an amino acid differing in identity from the amino acid found in a corresponding position in a wild-type polypeptide sequence.

A “mutant” or “engineered” GTPase is a GTPase having one or more substitutions in the amino acid sequence (e.g., non-native amino acids) as compared to the corresponding reference sequence. Each substitution in sequence is denoted as “XnnnY,” where X represents the original amino acid, nnn represents the position of the amino acid in a reference (e.g., wild-type or parental) sequence, and Y represents the replacement amino acid.

The term “orthogonal” typically refers to one or more changes, modifications and/or mutations in a composition which can be used to confer different or additional properties to a system. Orthogonal activity modulators, or orthogonal modulators, include compounds having one or more chemical moieties or structures that render the modulator unable to bind to a wild-type target protein, while still retaining discriminatory binding and/or modulatory activity versus a mutant protein. From another perspective, the structural changes present in the orthogonal modulators provide an additional property of “not binding” to a wild-type protein, while retaining their primary ability (binding to the mutant protein). In the context of the present invention, the “orthogonal GTPase modulators” of the present invention includes any number of GTPase inhibitor and/or activator compositions modified in a manner that substantially reduces or eliminates their ability to bind to the wild-type target GTPase molecule.

As used herein, the term “effector molecule” refers to a component, such as protein, enzyme or cofactor, which propagates a GTPase-dependent activity or signal upon activation of the GTPase (e.g., upon GTP binding or dephosphorylation). For example, effector molecules such as adenylate cyclase and phospholipase C generate chemical messengers (e.g., cyclic AMP and inositol phosphate) via interaction with a GTPase-GTP complex, while effector molecules such as the kinase c-Raf-1 propagate a signal through phosphorylation of proteins further downstream in the signaling pathway. Other GTPase binding molecules, such as GDI and GEF act as upstream effectors of GTPases.

The term “gene expression profile” refers to a data set generated for a plurality of genes in a cell or tissue; the data set typically reflects changes in genetic expression with varying conditions.

### BRIEF DESCRIPTION OF THE DRAWINGS

FIG. 1 provides a three-dimensional depiction of wild-type H-Ras, an exemplary GTPase.

FIG. 2 depicts exemplary scaffolds used in the design of the GTPase modulators of the present invention.

FIG. 3 depicts exemplary modulators based upon the purine ring structure of guanine.

FIG. 4 depicts exemplary modulators based upon a guanosine scaffold.

FIG. 5 depicts exemplary modulators based upon a GTP scaffold.

FIG. 6 depicts exemplary modulators based upon a GDP scaffold.

FIG. 7 depicts exemplary modulators based upon a GMP scaffold.

FIG. 8 depicts one embodiment of a Ras modulator (inhibitor) assay.

FIG. 9 is a schematic representation of the relative positions of the amino acid sidechains proximal to the GTP binding site of H-Ras, including the gatekeeper amino acids L19 and N116 (panel A) and the L19A-N116A double mutant (panel B).

FIG. 10 provides a bar chart showing the ability of various guanine-scaffold modulators and control molecules to replace radiolabeled GDP bound to the double mutant L19G-N116G.

FIG. 11 provides a bar chart showing the ability of various GTP-scaffold modulators and control molecules to replace radiolabeled GDP bound to the double mutant L19A-N116A.

FIG. 12 provides a bar chart showing the ability of various GTP-scaffold modulators and control molecules to replace radiolabeled GDP bound to wild-type (panel A) and N116G mutant GTPase (panel B).

FIG. 13 provides a bar chart showing the ability of various GDP-scaffold modulators to replace radiolabeled GDP bound to wild-type (panel A) and L19A-N116A mutant GTPase (panel B).

FIG. 14 panels A-C provide a schematic representation of the position of GTP binding site substitutions N116C (FIG. 9A), T144C (FIG. 9B) and L19C (FIG. 9C) with respect to the purine ring of a modulator structure.

FIG. 15 provides a bar chart showing the ability of various guanosine-scaffold modulators to replace radiolabeled GDP bound to the mutant N116C.

FIG. 16 provides a bar chart showing the ability of various GMP-scaffold modulators to replace radiolabeled GDP bound to the double mutant N116G-T144C.

FIG. 17 provides a bar chart showing the ability of various GMP and GTP-scaffold modulators to replace radiolabeled GDP bound to the mutant N116C.

FIGS. 18 through 31 provide various synthetic schemes for preparation of the GTPase modulators of the present invention or related synthetic intermediates.

FIG. 32 schematically illustrates the “switching” on and off of a GTPase by substrate.

FIGS. 33A and 33B are bar graphs depicting the selective binding of compounds 52 and 60 to mutant Ras and Rap1B proteins, respectively.

FIG. 34 is a line graph illustrating inhibition curves (IC₅₀) for GDP and Compound 60 binding to mutant (19A-116A) Ras.

FIG. 35 is a western blot illustrating inhibition and activation of mutant (19A-116A) Ras by modulators as measured by effector binding.

FIGS. 36A and 36B schematically illustrate the modified binding sites of wild type H-Ras bound to GDP and of mutant (19A-116A) Ras bound to compound 60, respectively.

FIG. 37 provides a three-dimensional depiction the interactions between a pyridine-containing modulator compound of the present invention (compound 96) and select Ras GTPase amino acid residues.

### DETAILED DESCRIPTION

The present invention provides modified GTPase sequences, cell lines expressing mutants GTPases, and GTPase activity modulators (“GTPase modulators” or “modulators”), e.g., inhibitors and/or activators, that bind preferentially to the modified GTPase, optionally altering (i.e., modulating) the activity of the GTPase. The mutant GTPase proteins and activity modulators of the present invention offer considerable value in the analysis and modulation of GTPase activity.

GTPase proteins can be engineered with modifications in the GTP binding site, such as changes in access to the substrate cavity, enlarged cavities, changes in the electrostatic or hydrophobic nature of the cavity, substitution or insertion of a nucleophilic residue (e.g., cysteine), and the like. Furthermore, specific GTPase modulators can be synthesized to modulate any suitably-engineered GTPase. Due to the highly conserved nature of GTPase sequences coding for the active site, the substrate pocket engineering scheme (e.g., the mutational approach) of the present invention can be applied to any GTPase of interest. These activity modulators can be used, for example, to elucidate the signaling response pathway(s) controlled by the modulated GTPase in, e.g., a global gene expression monitoring system exposed to various different stimuli, in in vitro effector binding assays, etc. Alternatively, the engineered proteins can be used as pharmaceutical products to remedy the deleterious effects of one or more malfunctioning or disease-producing wild-type GTPases. Thus, the GTPase modulators and engineered proteins of the present invention can be an extremely potent tool for deciphering and manipulating a desired GTPase activity.

### GTPase Families

GTPases are enzymes that use GTP binding and hydrolysis to drive the sequential binding of molecules to a series of reaction partners. Numerous GTPases are known in the art (see, e.g., Venter et al. (2001) Science 291: 1304-51, Bourne et al. (1991) Nature 349:117-127, which are incorporated herein by reference in their entirety), any of which provide a suitable basis for the compositions and methods of the present invention. There are three main families of GTPases: the G, subunits of the heterotrimeric G proteins (e.g., G_{S}, Gᵢ; see, for example, accession number pf00503 in the pfam database), the translation elongation factors involved in protein synthesis (e.g., EF-Tu; see, for example, accession number pf00009 in the pfam database), and the low molecular weight GTPases (Ras, Rho, Rab, Ran, Kir, Gem, Sar1/Arf, and the like; see, for example, accession number pf00071 in the pfam database).

GTPases have been shown to be involved in a number of important cellular processes, including, but not limited to, protein biosynthesis, translocation processes in the endoplasmic reticulum, vesicle traffic, control of cell differentiation, proliferation and oncogenesis, T-cell activation, and transmembrane signal transduction. GTPase activity is also associated with dynamin and tubulin, structural proteins involved in movement of intracellular components. For example various members of the Ras superfamily (Accession number pf00071 in the pfam database) including the Ras, Rab, Rac, Ral, Ran Rap and Ypt1 sub-families, among others, reflect a variety of these functions in vivo. Members of the Ras subfamily (Accession no. smart00173), e.g., p21 Ras, are involved in coupling GTPases to receptor tyrosine kinases. Members of the Rab subfamily (Acc. Nos. cd00154, smart00175), of which at least 60 have been identified in the human genome, are implicated in vesicle trafficking. In the GTP-bound form, the Rab GTPases recruit specific sets of effector proteins onto membranes, thereby regulating vesicle formation, actin- and tubulin-dependent vesicle movement, and membrane fusion. The Rho subfamily (Acc. Nos. cd00157, smart00174), including Cdc42 and Rac, as well as Rho isoforms are involved in the reorganization of the actin cytoskeleton in response to external stimuli. Members of the Rho subfamily have also been shown to play a role in cell transformation by Ras, in cytokinesis, in focal adhesion formation and in the stimulation of stress-activated kinase. Each of these functions is controlled through distinct effector proteins and mediated through a GTP-binding/GTPase cycle involving three classes of regulating proteins: 1) GTPase-activating proteins (GAPs); 2) guanine nucleotide exchange factors (GEFs); and 3) guanine nucleotide dissociation inhibitors (GDIs). The Ran GTPase, a member of the Ran/TC4 subfamily of small GTPases (Acc. No. smart00176) has been shown to be involved in the active transport of proteins through nuclear pores. In addition, the GTPase SAR 1 (Acc. No. COG1100) and related proteins are predicted to have GTPase function. Full length GTPase sequences (e.g., selected from among P01115, P23175, P01113, P01114, CAA25322.1, AAA46570.1, NP_{—}0005334.1, AAA72806.1, NP_{—}032310.1, P20171, P08642, A43816, AAB21190.1, 1604384A, AAK64517.1, BAB61870.1, BAB61869.1, 2Q21, 1Q21, 821P, 421P, P08556, NP_{—}035067.1, 1PLK, 1AGP, BAB27790.1, NP_{—}002515.1, P12825, 1LFD, NP_{—}5429441, 1RVD, 5P21, AAD56718.2, 621P, AAK84038.1, 1LF0, 0909261A, 1IAQ, 221P, 721P, 521P, 1NVV, 2105181A, NP_{—}203524.1, P32883, Q91806, P01117, CAA73253.1, AAB41942.1, AAB97888.1, CAA80675.1, P05774, S31720, NP_{—}004976.2, P79800, AAP36888.1, AAH13572.1, CAA76678.1, A37355, O42277, Q9YH38, Q07983, NP_{—}113703.1, A54321, NP_{—}571220.1, NP_{—}067259.2, Q05147, AAC25633.1, S34138, AAC35360.1, AAH50837.1, CAA76779.1, AAF17287.1, AAH48875.1, AAF17286.1, AAH06499.1, NP_{—}476699.1, 1306284A, XP307965.1, 180324, P18262, AAK14389.1, S35097, AAB190641, P22981, NP_{—}502213.2, I59431, AAB09439.1, XP_{—}215123.1, AAA83022.1, CAD24769.1, AAA49429.1, 1707302A, P15064, P34729, P34726, P51539, JC6328, S58220, and P03967, among many others readily ascertainable, e.g., via the world wide web at ncbi.nlm.nih.gov, to those of skill in the art), polypeptide fragments, and fusion constructs can all function as GTPase sequences in the present invention. Notably, compositions and methods of the present invention can be extended to these or any other GTPase of interest.

Although the GTPases are employed in a variety of diverse functions, they are characterized by highly conserved sequence motifs (i.e., shared structural characteristics that define the genus) that encode the guanine nucleotide-binding domain (see, for example, Bourne et al. (1991) “The GTPase superfamily: conserved structure and molecular mechanism” Nature 349:117-27). For example, Table 1 illustrates a partial alignment of 9 exemplary GTPases showing amino acid subsequences which comprise part of the substrate binding site.

TABLE 1
Partial amino acid sequence alignment of exemplary GTPases.

| | | amino acid | | amino acid |
|---|---|---|---|---|
| GTPase | position | subsequence | position | subsequence |
| H-Ras | 10 | GAGGVGKSALTI | 113 | LVGNKCD |
| Rab7 | 15 | GDSGVGKTSLMN | 122 | VLGNKID |
| Ran | 17 | GDGGTGKTTFVK | 119 | LCGNKVD |
| Ra1B | 21 | GSGGVGKSALTL | 125 | VVGNKSD |
| Rap1B | 10 | GSGGVGKSALTV | 113 | LVGNKCD |
| RhoA | 12 | GDGACGKTCLLI | 114 | LVGNKKD |
| Arf4 | 27 | GLDCAGKTTVLY | 131 | IVANKQD |
| Gₜ | 40 | GAGESGKSTIVK | 266 | LFLNKKD |
| EF1A | 14 | GHVDSGKSTTTG | 150 | VGVNKMD |

Alignment of additional members of the GTPase superfamily can be found, e.g., in Bourne et al. (1991) Nature 349:117-127, and on the world wide web. For example, an alignment of all currently known members (1554) of the Ras family of GTPases is available on the world wide web at pfam.wustl.edu/cgi-bin/getdesc?acc=PF00071. Extension of these partial alignments as well as (partial or complete) alignment of additional members of the GTPase superfamily can be performed by one of skill in the art, without undue effort or experimentation, using available search and alignment algorithms, such as the BLAST algorithm (described in Altschul et al. (1990) J. Mol. Biol. 215:403-410). BLAST is publicly available along with extensive databases of nucleotide and amino acid sequences, including the complete human genome sequence, e.g., through the National Center for Biotechnology Information on the world wide web at ncbi.nlm.nih.gov. Alternatively, one of skill in the art can perform manual or computer assisted alignments using the local homology algorithm of Smith and Waterman (1981) Adv. Appl. Math. 2:482; the homology alignment algorithm of Needleman and Wunsch (1970) J. Mol. Biol. 48:443; the search for similarity method of Pearson and Lipman (1988) Proc. Natl. Acad. Sci. (USA) 85:2444; and/or by computerized implementations (e.g., GAP, BESTFIT, FASTA, and TFASTA in the Wisconsin Genetics Software Package Release 7.0, Genetics Computer Group, 575 Science Dr., Madison, Wis.). Another example of a useful sequence alignment algorithm is PILEUP. PILEUP creates a multiple sequence alignment from a group of related sequences using progressive, pairwise alignments. PILEUP uses a simplification of the progressive alignment method of Feng & Doolittle (1987) J. Mol. Evol. 35:351-360. The method used is similar to the method described by Higgins & Sharp (1989) CABIOS5:151-153. An additional example of an algorithm that is suitable for sequence alignments is the CLUSTALW program (Thompson, J. D. et al. (1994) Nucl. Acids. Res. 22: 4673-4680).

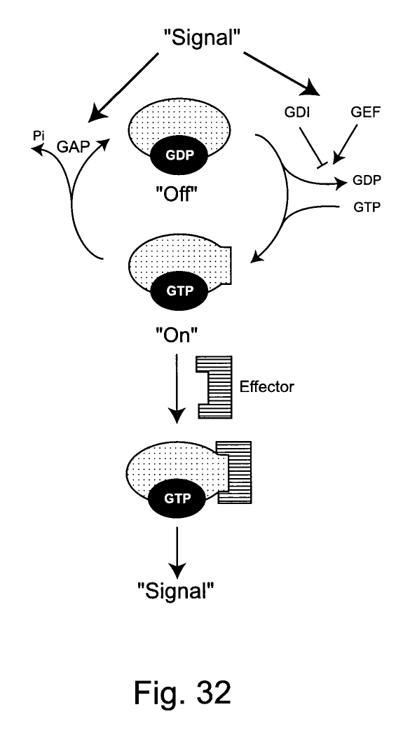

The GTPases have picomolar binding affinities for GTP and GDP. Generally, nucleotide binding acts as a “switch” for protein activity (such as recruiting one or more downstream effectors, thereby turning “on” or “off” a signaling cascade). Generally, the protein is turned “on” by the binding of GTP and turned “off” again by the hydrolysis of GTP to GDP, as illustrated schematically in FIG. 32. Cycling between these two states is tightly regulated in the cell, by both GTPase activating proteins (GAPs) that enhance the rate of GTP-hydrolysis, guanosine exchange/release factors (GEFs and GRFs) that accelerate the GDP/GTP exchange, and guanosine dissociation inhibitors (GDIs) that slow the GDP/GTP exchange. However, if the switch control mechanisms of the GTPase molecule are impaired (by being “frozen” in either the ON or OFF position), a number of different diseases, including cancers, can ensue.

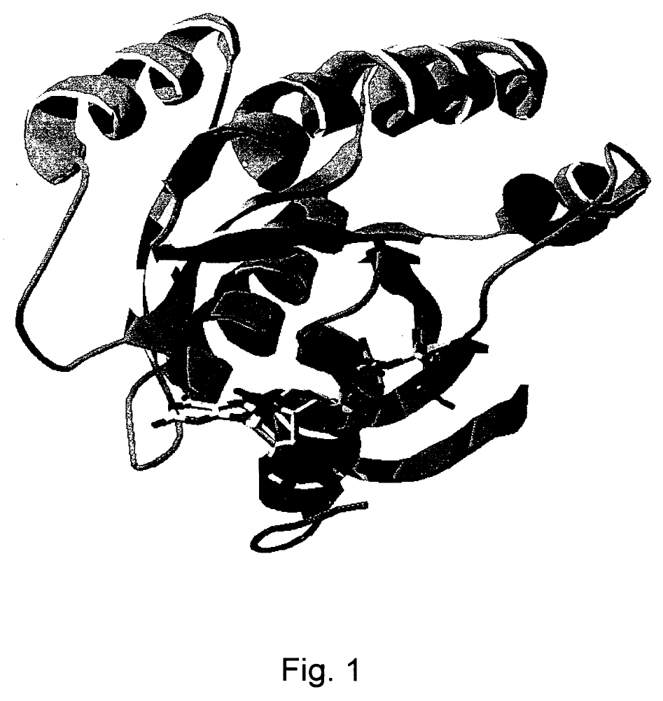

The prototypical GTPase protein to which most putative or newly-discovered GTPase sequences are compared is Ras (FIG. 1). Ras acts to transduce information from the cell surface to the nucleus. For example, Ras couples growth signals from receptor tyrosine kinases to the mitogen activated protein kinase cascade, one mechanism of cell proliferation control (or, when misregulated, a means for malignant transformation).

The mammalian Ras genes encode a set of relatively small (19-21 kD) proteins typically having between 170-189 amino acids. There are at least four classes, or types, of Ras protein: H-Ras, K-(A)-Ras, K-(B)-Ras, and N-Ras, each corresponding to a different gene and whose protein products may have different roles in cells, see, for example, Wolfman (2001) “Ras Isoform-Specific Signaling: Location, Location, Location” Science STKE 96:PE2. The three dimensional structure of H-Ras has been elucidated in the presence of both GDP and GTP analogues; as such, H-Ras provides an excellent base upon which to rationally design mutant GTPases.

Ras genes were first identified as the genes responsible for cellular transformation (e.g., oncogenesis) in murine sarcoma viruses. Deregulated Ras GTPase activity has been found to be present in 30 to 60% of all tumor cell types. However promising it could be, cancer treatment through direct Ras inhibition has proved out of reach due to the near impossibility of synthesizing small molecule inhibitors able to compete with GDP and GTP (See, for example, Downward (2003) “Targeting Ras signaling pathways in cancer therapy” Nat. Rev. Cancer 3:11-22; Bos (1989) “Ras oncogenes in human cancer: a review” Cancer Res. 49:4682-4689; Gunzburg (1999) “Proteins of the ras pathway as novel potential anticancer therapeutic targets” Cell Biol. Toxicol. 15:345-358 and references cited therein).

GTPases have maintained a highly conserved sequence encoding the guanine nucleotide binding domain (also referred to as “ligand binding site” or “substrate pocket”). Typically, the substrate pocket is a relatively elongated and narrow cavity along the protein surface. In the GTPase H-Ras, the back of the substrate pocket is delineated by gatekeeper residues L19 and N116. The side chains of these residues are proximal to the substrate binding cavity, and concomitantly impede access of the ligand (i.e., GTP, GDP, or a modulator molecule) to an hydrophobic pocket located near the binding site. Mutational analysis has shown that point mutations in a number of amino acids (e.g., residues 12, 13, 28, 59, 61, 63, 116, 117, 119, 144 and 146) lead to constitutive activation of the Ras protein along with either reduced phosphatase activity (i.e., longer bound times for GTP) or decreased nucleotide affinity (i.e., increased exchange of bound GDP for cytosolic GTP). As might be expected, these amino acids are positioned proximal to the guanine nucleotide binding site. Molecular modeling studies indicate that residues 10-16 and 56-69 interact with the phosphate moieties of the ligand, while residues 116-119 and 152-165 interact with the guanine base portion of the molecule. (See, for example, Bourne et al., supra; Barbacid (1990) “Ras oncogenes: their role in neoplasia” Eur. J. Clin. Invest. 20:225-235; Polakis and McCormick (1993) “Structural requirements for the interaction of p21^{ras} with GAP, exchange factors, and its biological effector target” J. Biol. Chem. 268:9157-9160; Walter et al (1986) “The oncogenic activation of human p₂₁^{ras} by a novel mechanism” Science 233:649-652; Sprang (1997) “G proteins mechanisms: insights from structural analysis” Annual Rev. Biochem. 66:639-678, and references cited therein).

### Modification of Wild-Type GTPases

In one aspect, the present invention provides engineered, or “mutant” GTPases, as well as the amino acid and nucleic acid sequences encoding the mutant proteins. GTPase sequences which can be modified to generate the mutant GTPases of the present invention include, but are not limited to, members of the Ras (H-Ras human: AF493916, mouse: Z50013), Rho (RhoA human: AF498970, mouse: AF014371), Rab (Rab7 human: X93499, mouse: X8950), Ran (human: M31469, mouse: AAA64248), and Sar1/Arf (Arf4 human: U73960, mouse: U76546) families. Members of the heterotrimeric G protein alpha subunit category such as Gₛ (human: X04408, mouse: Y00703), Gₜ (human: D10384, mouse: L10666), Gᵢ (human: X04828, mouse: M13963), Gₒ (human: M60162, mouse: M36778) are also included. Indeed, as indicated above, the compositions and methods of the present invention are applicable to any GTPase. For example any human GTPase, such as those enumerated in Bourne et al. (1991) Nature 349:117-127, or readily ascertainable by entering any of the above accession numbers into an available web based search tool, e.g., using ENTREZ at ncbi.nlm.nih.gov), is a suitable starting point for making the mutant GTPases of the present invention. It should be noted that, in addition to complete protein sequence, peptide fragments having GTPase activity (e.g., partial protein sequences), are also contemplated in the present invention.

Standard analytical and molecular modeling techniques can be utilized to identify and/or confirm amino acids proximal to the GTP binding site in the wild-type GTPase selected for modification. For example, three-dimensional configurations can be determined by any of a number of different analytical techniques, including X-ray crystallography, nuclear magnetic resonance (NMR), and homology-based modeling programs such as INSIGHTII (Biosym Technologies, San Diego, Calif.). Alternatively, the mutant form of the wild-type GTPase can be designed by reference to a 3-dimensional model or crystal structure of the wild-type protein (if available), or of a second GTPase protein similar in sequence (e.g. having some sequence identity with) or homologous (having a similar genetic ancestry) to the wild-type protein.

Once the GTPase of interest is selected, and the amino acids proximal to the GTP binding site are identified or confirmed, mutant GTPase proteins can be generated and assessed for their ability to interact with one or more activity modulators. The mutation(s) in the GTPase amino acid sequence are generated by any of a variety of synthetic methodologies well known to those skilled in the art, including, but not limited to: site directed mutagenesis of a nucleic acid sequence encoding the wild-type protein, various PCR methodologies such as “error prone” PCR and “sexual” PCR, recursive recombination (such as described by Stemmer in U.S. Pat. Ser. No. 6,372,497, “Methods for generating polynucleotides having desired characteristics by iterative selection and recombination”), and other combinatorial methodologies. Either single amino acids or combinations of amino acids can be altered in the wild-type (or mutant) GTPase, thereby modifying (or further modifying) the ligand binding site.

Preferably, the selected wild-type GTPase sequence is modified to create mutant GTPases with modified GTP/GDP binding pockets. In some embodiments of the present invention, the binding pocket is enlarged (e.g., by substituting amino acids having smaller side chains), allowing the mutant GTPase to accommodate modulator molecules having one or more bulky substituents. Alternatively, the polarity or hydrophobicity of the binding site can be altered by substituting appropriate amino acids. Optionally, the amino acids proximal to the back side of the substrate pocket (i.e., the gatekeeper residues) are modified, thus altering the depth of the substrate pocket. In an embodiment, the mutant GTPase is created having one or more amino acids positioned in or near the substrate binding pocket that are capable of forming a covalent bond with a GTPase modulator of the present invention.

In one embodiment of the present invention, a single amino acid is altered in the GTPase sequence. For example, an amino acid correlating in position to L19, F28, N116, K117 or T144 of the H-Ras sequence is mutated to a different amino acid. In other embodiments, combinations of these amino acids (e.g., sets of two, three, or more amino acids) are modified to produce the mutant GTPase. While the amino acid to be altered is preferably mutated to an alanine, glycine, or cysteine residue, other amino acid substitutions are also considered. The amino acid and nucleic acid sequences of H-Ras are provided as SEQ ID Nos. 1 and 18, respectively. Exemplary mutant amino acid sequences are provided in SEQ ID No. 2-17; the corresponding nucleic acid sequences are provided in SEQ ID No. 19-34.

The present invention provides both mutant amino acid sequences as well as the corresponding nucleic acid sequence. Due to the inherent degeneracy of the genetic code, other nucleic acid sequences that encode substantially the same or a functionally equivalent amino acid sequence can also be used to generate the GTPase polypeptides. As will be understood by one of skill in the art, it can be advantageous to modify a coding sequence to enhance its expression in a particular host cell. While the genetic code is redundant with 64 possible codons, most organisms preferentially use a subset of these codons. The codons that are utilized most often in a species are called optimal codons, and those not utilized very often are classified as rare or low-usage codons (see, for example, Zhang et al. (1991) Gene 105:61-72). Codon optimization can be used, for example, to increase the rate of translation, or to produce recombinant RNA transcripts having desirable properties (such as a longer half-life). Translation stop codons can also be modified to reflect host preference. The engineered GTPase-encoding sequences of the present invention also include silent codon substitutions reflecting the preferred codon usage of the host (e.g. mutated sequences having undergone “codon optimization” are also contemplated in the present invention).

The mutant GTPases of the present invention can be produced from the corresponding mutated nucleic acid sequences using any of a number of different approaches known to one of skill in the art, including, but not limited to, in vitro translation methodologies, as well as cloning and expression technologies. The nucleic acid sequences encoding the mutant GTPase can be various forms of deoxyribonucleic acid (for example, genomic DNA or cDNA) or ribonucleic acid (e.g., messenger RNA). Numerous cloning and in vitro amplification methods suitable for the construction of recombinant nucleic acids sequences are well-known to persons of skill. General texts which describe molecular biological techniques useful in the production of the mutant GTPase-encoding sequences include Berger and Kimmel, “Guide to Molecular Cloning Techniques”, Methods in Enzymology, volume 152 Academic Press, Inc., San Diego, Calif.; Sambrook et al., Molecular Cloning—A Laboratory Manual (2nd Ed.), Vol. 1-3 (1989, Cold Spring Harbor Laboratory, Cold Spring Harbor, N.Y.); F. M. Ausubel et al., eds., Current Protocols in Molecular Biology (a joint venture between Greene Publishing Associates, Inc. and John Wiley & Sons, Inc., supplemented through 2000); and Innis et al., eds. PCR Protocols A Guide to Methods and Applications, Academic Press Inc. San Diego, Calif. (1990).

### Generation of Exemplary Mutant Enzymes

In one embodiment of the present invention, Ras protein was selected as the GTPase prototype. The nucleic acid sequence of wild-type H-Ras (AC number J00277) was cloned into a pGEX vector (Pharmacia Corporation, New Jersey) having a glutathione-S-transferase (GST) gene fusion sequence under a tac promoter. Mutations in the GTPase nucleic acid sequence were generated using the QuikChange® site-directed mutagenesis protocol from Stratagene (La Jolla, Calif.).

In brief, primers having the desired codon change were used to PCR amplify the pGEX vector containing the wild-type sequence, giving rise to plasmids that incorporate the changed base. DNA polymerase was used to extend the mutagenic primers under high-fidelity, non-strand displacing conditions. While the wild-type-containing plasmid will contain methylated DNA, the newly synthesized sequences will not be methylated, providing a mechanism for removal of the original plasmid. The thermal cycling reaction products were treated with restriction endonuclease Dpn I to digest the parental DNA template. This mixture was then transformed into E. coli cells, where the single stranded mutant DNA sequences were converted into duplex form in vivo. Double stranded plasmid DNA was prepared from the transformants and analyzed to identify clones bearing each of the desired mutations.

The expression vector pGEX contains a strong, inducible tac promoter that follows the lac operon regulation mechanism. Mutant Ras cDNAs were transformed individually in BL21-Gold Competent Cells™ from Stratagene according to the manufacturer's instructions. An overnight culture of BL21-Gold cells containing one of the GST-H-Ras plasmids described above was diluted 1:50 in Superbroth™ medium (Q-Biogene, Carlsbad, Calif.) supplemented with 50 mg/L carbenicillin (Sigma-Aldrich, St. Louis, Mo.). After it had grown to an OD=0.6, synthesis of the GST-H-Ras protein was induced by the addition of 50 μM isopropyl-β-D-thiogalactopyranoside (Promega, Madison, Wis.) and by further incubation at 30° C. for 12 h. After centrifugation at 3,500 g for 15 min at 4° C., the cell pellet was frozen at −80° C. for 1 h. The bacterial cell pellet was then resuspended in lysis buffer (20 mM HEPES pH 7.5, 75 mM KCl, 25 mM MgCl₂, 5 mM DTT, 0.1 mM EDTA, 0.05% Triton X-100, 5 mM benzamidine, 10 mg/L aprotinin, 10 mg/L antipain, 10 mg/L pepstatin, 10 mg/L leupeptin and 1 mM phenylmethylsulfonyl chloride), chilled on ice and sonicated (Sonics, Vibracell) for 60 sec. The resulting lysate was centrifuged at 30,000 g for 30 min at 4° C., and the supernatant was added to glutathione sepharose beads (Pharmacia, #274574-01) for a 30 min incubation at 4° C. on a rotating wheel. The beads were washed with lysis buffer once, and then with wash buffer (50 mM Tris-HCl pH 7.5, 50 mM NaCl, 1 mM DTT) three times. The protein was eluted using 10 mM glutathione in wash buffer, concentrated and dialyzed overnight against dialysis buffer (50 mM Tris-HCl pH 7.5, 50 mM NaCl, 0.2 mM DTT, 0.1 mM EDTA). Protein concentration was determined by a Bradford assay and the protein purity was assessed by gel electrophoresis. The GTPase products were then tested for activity using ³H-GDP as substrate. Proteins kept at 4° C. conserved their activity for several weeks.

### Activity Modulators Specific to Modified GTPases

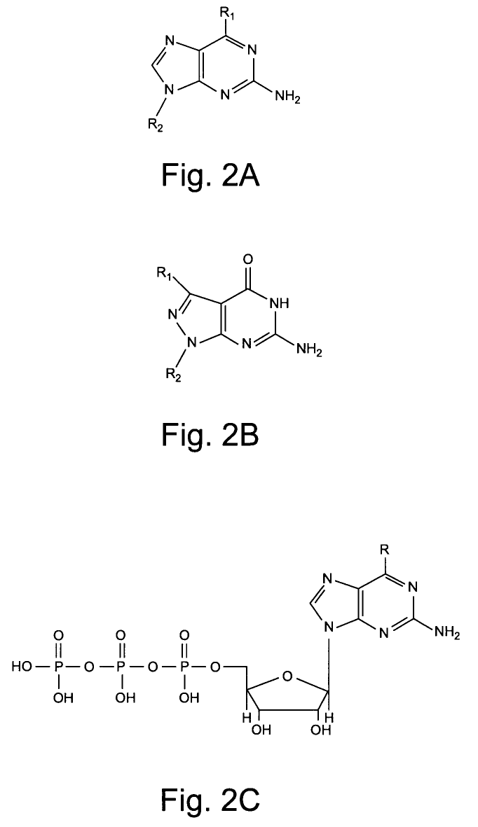

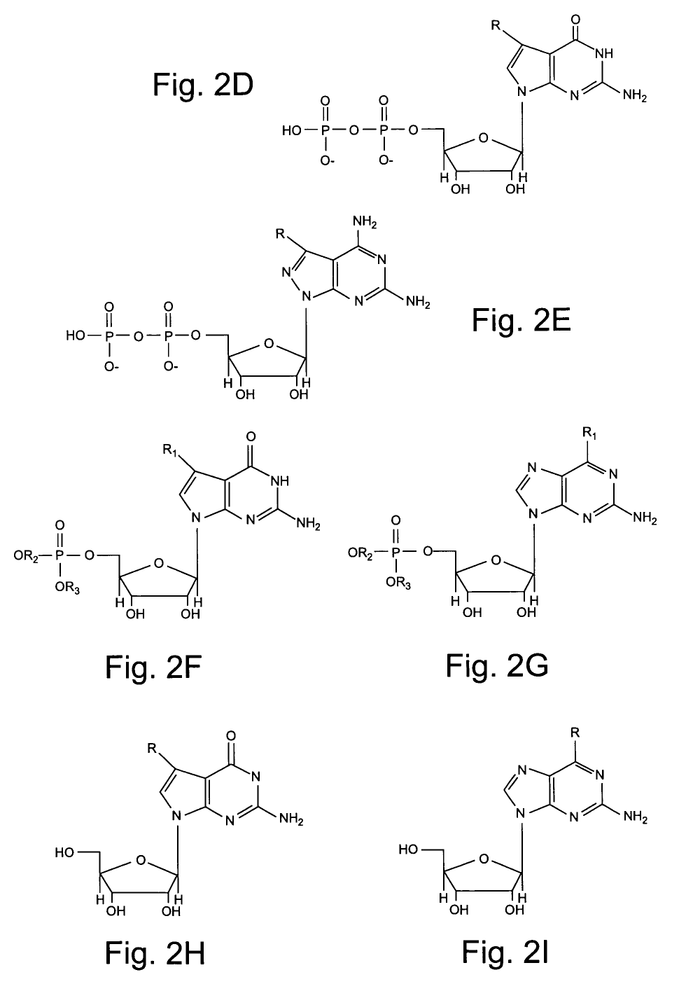

The present invention also provides inhibitors and activators of the modified GTPases and methods for their synthesis. Preferably, these activity modulators are “orthogonal” GTPase modulators with respect to the wild-type protein (i.e., the modulators are ineffective, or “silent,” in the presence of the wild-type GTPase). During the design of the orthogonal modulators, particular attention was directed to the use of natural ligands (GTP and related compounds) as scaffold structures, upon which a structural “bump” was formed, leading to one or more steric protuberances that affect the interaction with wild-type and/or mutant GTPases. Exemplary scaffold structures are provided in FIG. 2. While the modulator structures listed below are exemplary for each scaffold structure employed, the list is not exhaustive; the substituents listed for a given scaffold can be employed on any scaffold contemplated in the preset invention. In certain embodiments, the modulators, e.g., inhibitors and activators, are cell permeable, permitting selective perturbation of GTPase function in vivo. One of skill in the art will also recognize that additional similar substituents (e.g., isomeric structures, or substituents varying in the number of methyl “linker” units) can be substituted for one or more of those specifically listed. Methods for preparing the modulators of the present invention are disclosed in a following section.

### Structures Based Upon Guanine

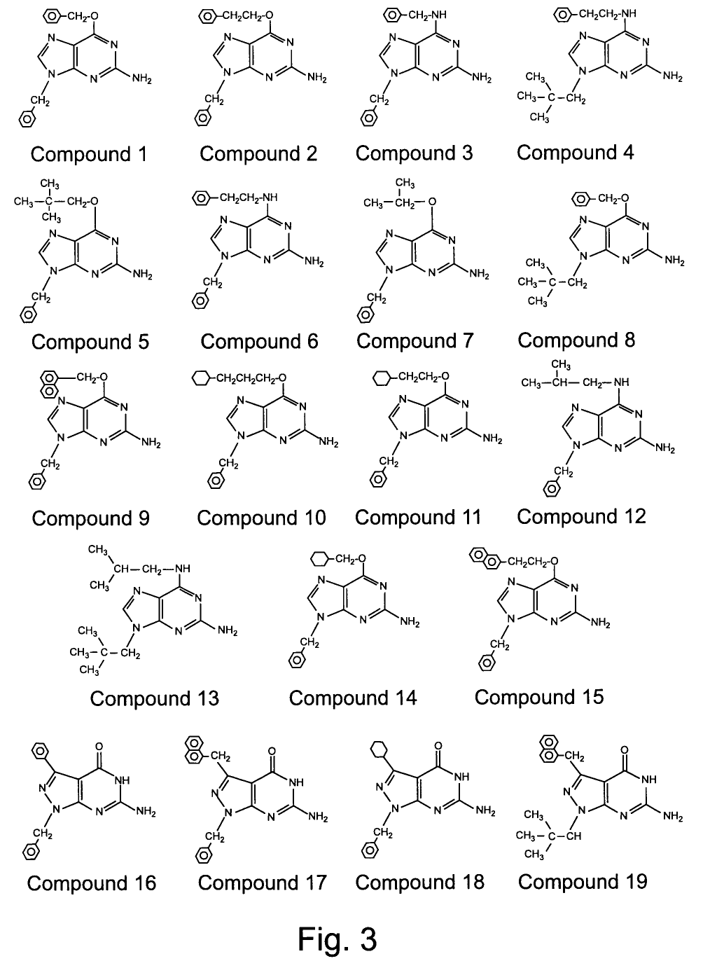

In one embodiment of the present invention, modulators are synthesized based upon guanine, the purine ring component of GTP. The modulators depicted in FIG. 3 are structurally based upon two different guanine scaffolds (FIG. 2, panels A and B). The R2 substituent coupled to the N-9 position (normally occupied by a ribose moiety in the GTP structure) is typically an alkyl group; exemplary R2 groups include, but are not limited to, a benzyl group (—CH₂C₆H₅) and a (2,2-dimethyl)propyl group (—CH₂C(CH₃)₃).

In the first guanine scaffold (panel A), the C-6 position is chemically modified with any of a number of bulky constituents R1, including, but not limited to, an O-propyl group, an O-isopropyl group, an O-isobutyl group, an O-sec-butyl group, an O-methyl-t-butyl group, an O-(2,2-dimethyl)propyl group, an O-cyclohexyl group, an O-methyl-cyclohexyl group, an O-(2-cyclohexyl)ethyl group, an O-(3-cyclohexyl)propyl group, an O-benzyl group, an O-(2-phenyl)ethyl group, an O-[2-(1-naphthyl)]ethyl group, or an O-[2-(2-naphthyl)]ethyl group. Alternatively, the R1 substituent can be linked through an amine linkage instead of an ester linkage (e.g., an N-isobutyl group, an N-benzyl group, an N-(2-phenyl)ethyl group, and the like).

In the second guanine scaffold provided in FIG. 2, the R2 position at N-9 is again typically occupied by an alkyl group such as a benzyl or (2,2-dimethyl)propyl group. However, the purine ring structure is further modified, such that the pentene ring N-7 is converted to a carbon atom. Furthermore, the pentene ring C-8 is optionally changed to a nitrogen atom (e.g., as in a pyrazolo-pyrimidine ring structure). Various chemical substitutions (R1) are present at the new C-7 position as provided for the previous scaffold at the C-6 position. For example, The C-7 position is chemically modified with any of a number of bulky constituents, including, but not limited to: a propyl group, an isopropyl group, an isobutyl group, a sec-butyl group, a t-butyl group, a (2,2-dimethyl)propyl group, a cyclohexyl group, a methyl-cyclohexyl group, a (2-cyclohexyl)ethyl group, a (3-cyclohexyl)propyl group, a phenyl group, a benzyl group, a (2-phenyl)ethyl group, a pyridine or pyridine derivative, a 3-pyrroline derivative, a [2-(1-naphthyl)]ethyl group, or a [2-(2-naphthyl)]ethyl group. Exemplary modulator structures based upon the guanine scaffold are provided in FIG. 3.

### Structures Based Upon Guanosine Scaffold

As an alternate approach, modulators are constructed based upon a guanosine (purine ring+ribose) scaffold. The C-6 position of the purine ring of guanosine is chemically modified with any of a number of bulky or reactive constituents, such as those provided above. In an embodiment, the C-6 substituent is a thiol or an electrophilic moiety capable of forming a covalent bond with the GTPase molecule. Thiol and electrophilic moieties considered for incorporation at the C-6 position include, but are not limited to, a [(3-maleimido)propylamido]ethyl group, a [(3-methyl)maleimido]ethyl group, a [(3,4-dimethyl)maleimido]ethyl group, a [(3,4-dimethyl)maleimido]propyl group, a (2-N-acrylamido)ethyl group, a [N-(4-fluorosulfonyl)benzamido]ethyl group, a sulfhydryl group, a substituted pyrroline moiety, or an alkyl sulfhydryl group (—O(CH₂)ₙ—SH, where n=1-4). Furthermore, the C-6 substituent can be either O-linked or N-linked as provided for the guanine ring scaffold.

In an additional embodiment, the C-6 position of the guanosine ring is in the ketone form, and the C-2 position of the purine ring is modified with one of the C-6 substituents.

The guanosine ring can have either a nitrogen atom at position 7 or be modified to contain a carbon atom at this position. The N-7 or C-7 is optionally substituted with any of a number of alkyl or aryl constituents, such as an isopropyl group, a tert-butyl group, a cyclohexyl group, a phenyl group, a p-fluorophenyl group, a benzyl group, a naphthyl group, an alkyl maleimide group (e.g., a (3-methyl)maleimide group, a (3,4-dimethyl)maleimide group, and the like), an acrylamide group (—NHCOCH═CH₂), an N-(4-fluorosulfonyl)benzamide group (—NHCOC₆H₄SO₂F), a pyridine moiety, a 3-pyrroline moiety, or other bulky substituents such as those contemplated for the C-6 position. In the embodiments having a carbon at position 7, the C-7 substituent is preferably coupled to the purine ring via an ethyl (—CH₂CH₂—) linkage or a sulfanyl (—CH₂S—) group.

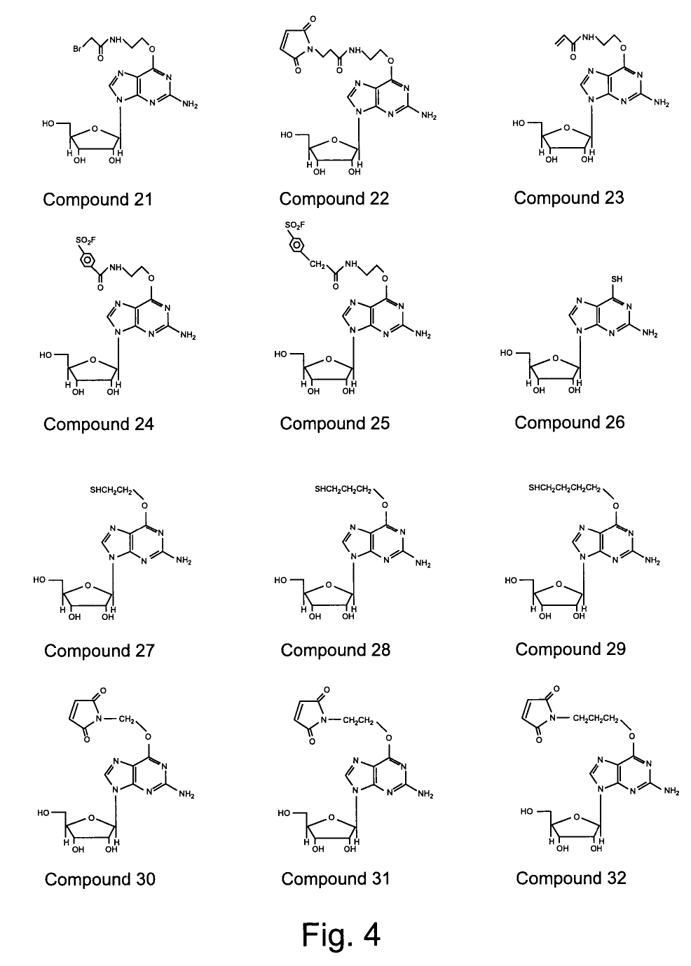

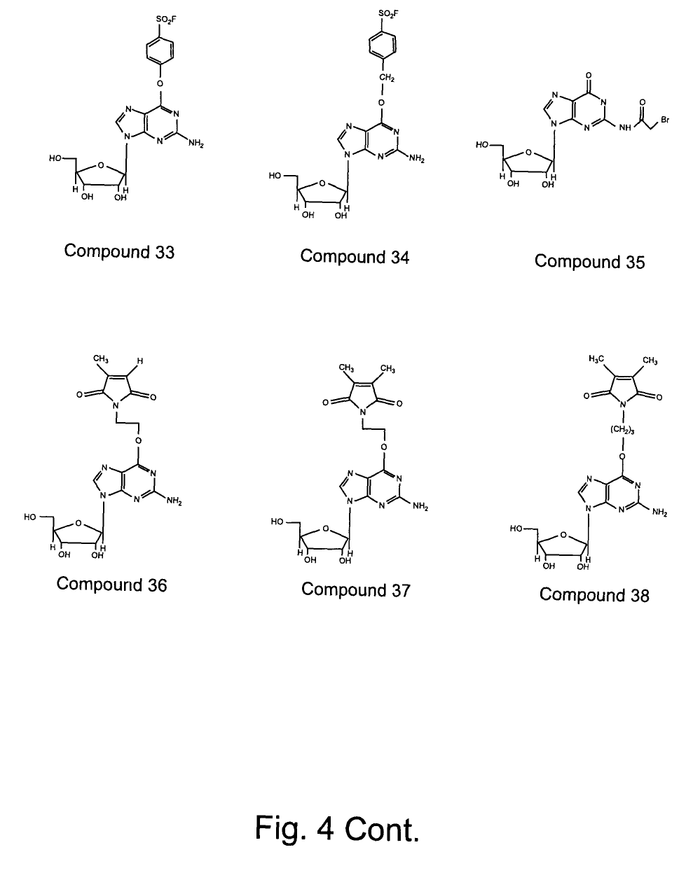

Exemplary modulator structures based upon the guanosine scaffold are provided in FIG. 4.

### Structures Based Upon Phosphorylated Guanosine Scaffolds

In another embodiment of the present invention, modulators are constructed based upon a phosphorylated guanosine scaffold (e.g., GTP, GDP or GMP). One advantage of retaining the ribose and phosphate groups lies in the additional binding affinity they can provide to the modulators. Since Ras binding of GTP and GDP is 10⁴ and 10⁵ better than the interaction with GMP and guanosine, respectively, retention of the ribose and phosphate groups is expected to improve the binding characteristics of the compound and provide highly potent modulators. The phosphate groups are engaged in multiple hydrogen bonds with the protein and a magnesium ion, explaining the much greater affinity of GTP or GDP versus GMP and guanosine, 10⁴ and 10⁵ respectively.

Any of the chemical modifications previously listed as C-6 or N-7/C-7 substituents of the guanine and guanosine scaffolds are also considered for use in generating phosphorylated guanosine-based modulators. For example, the C-6 position of the GTP scaffold can be chemically modified with any of a number of bulky O-linked or N-linked constituents listed herein. In an embodiment, at least one substituent at the 6-position or 7-position of the purine ring includes an electrophilic moiety (e.g., such as an O-linked [(3-maleimido)propylamido]ethyl group, an O-linked [(3-methyl)maleimido]ethyl group, an O-linked [(3,4-dimethyl)maleimido]ethyl group, an O-linked [(3,4-dimethyl)maleimido]propyl group, an O-linked (2-N-acrylamido)ethyl group, an O-linked (n-N-acrylamido)alkyl group, a pyridine moiety, a 3-pyrroline moiety, a thiol group, an alkyl thiol group, an alkyl halide group and the like). Typically, the (acrylamido)alkyl and alkyl thiol groups contain between one and three carbon atoms.

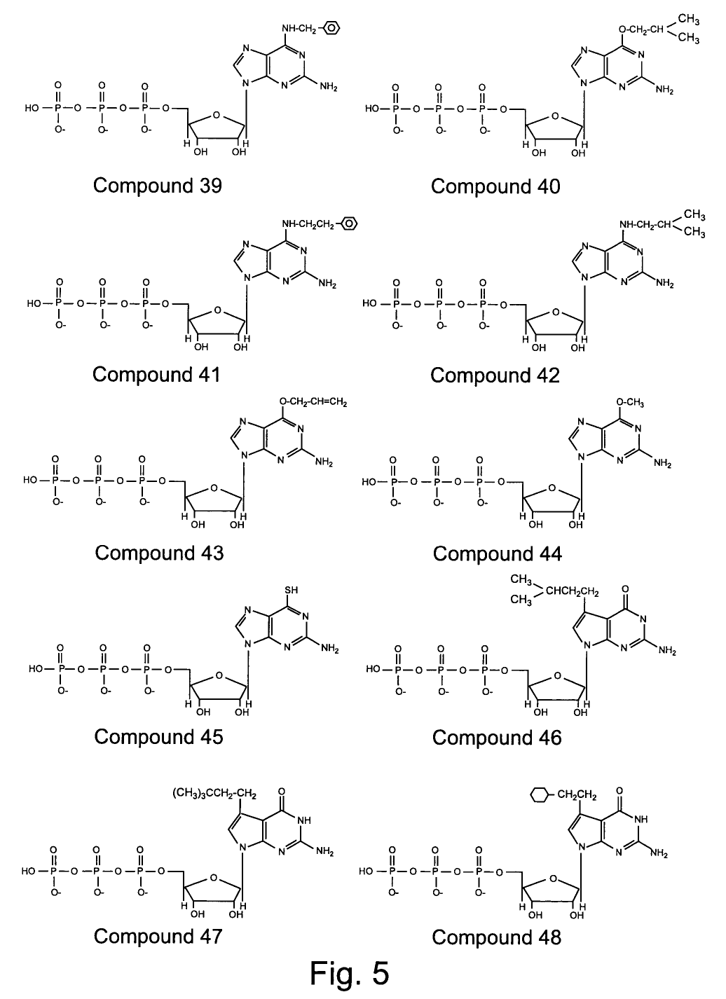

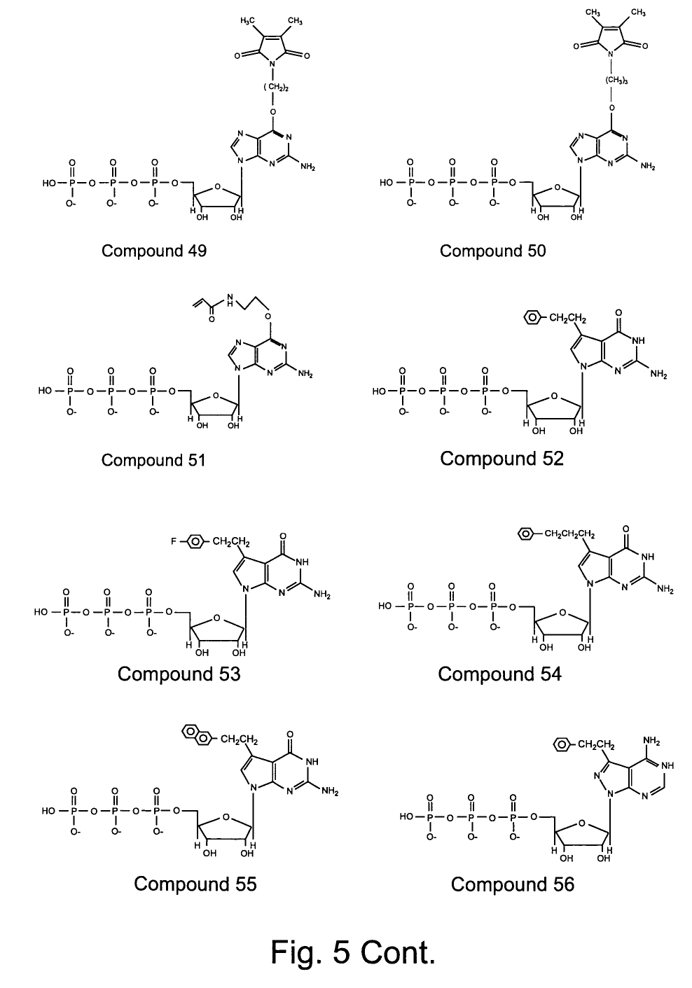

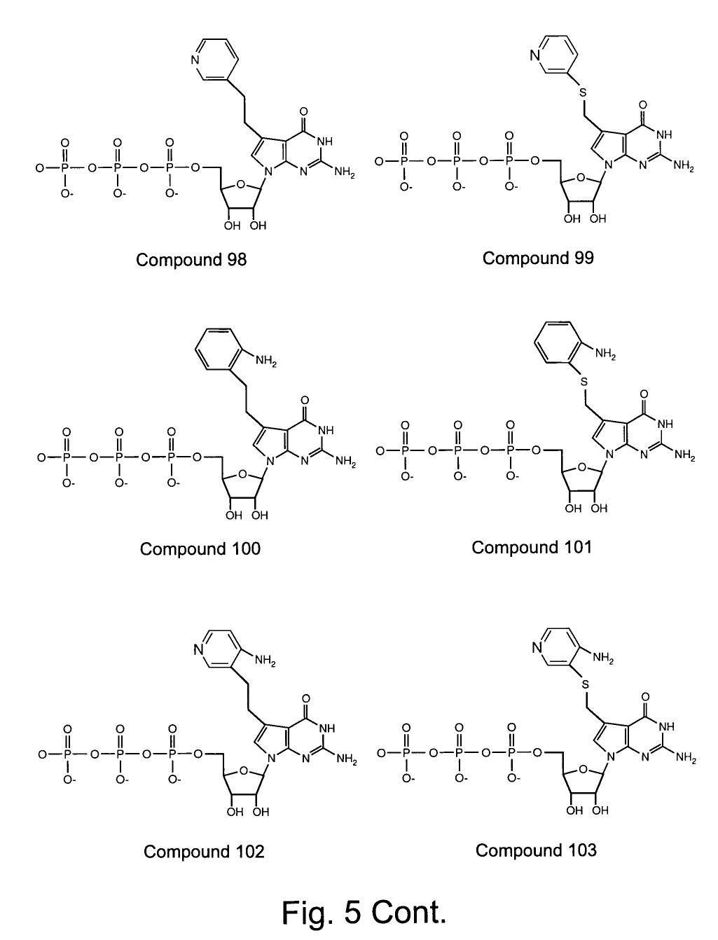

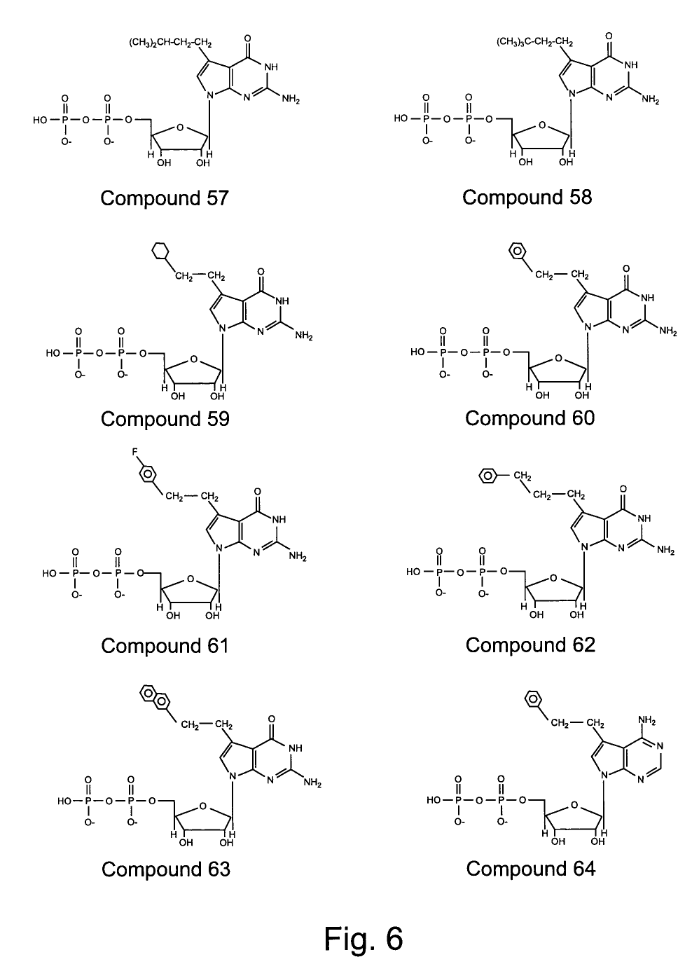

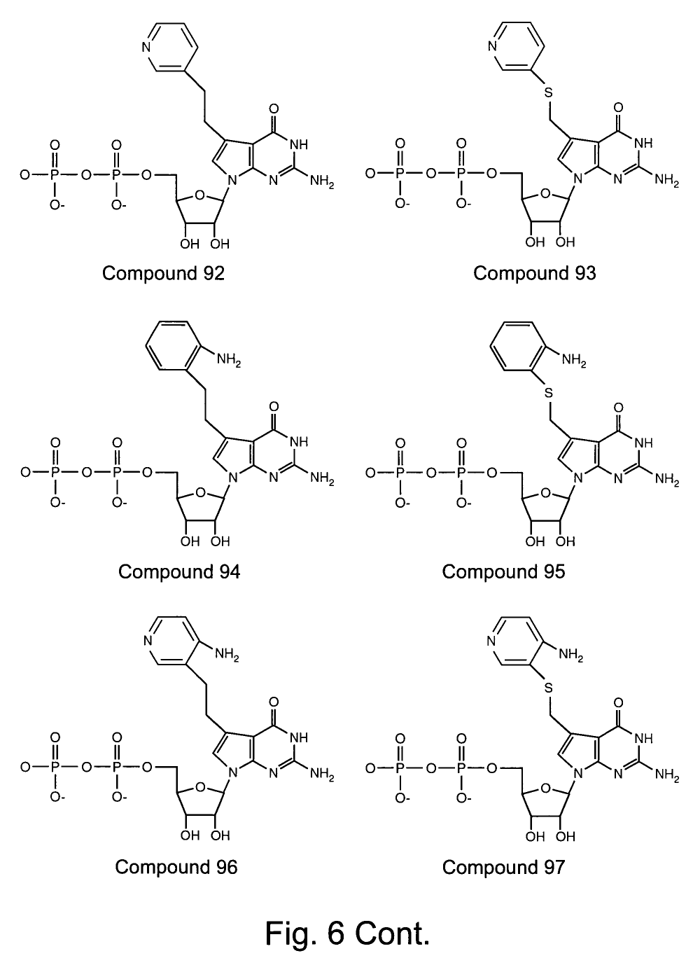

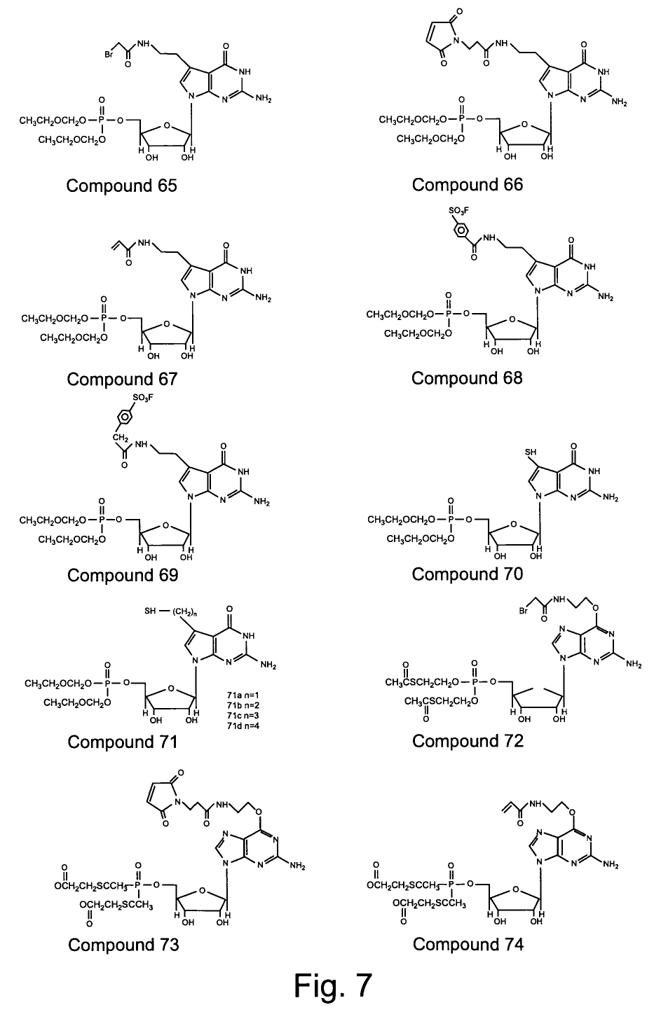

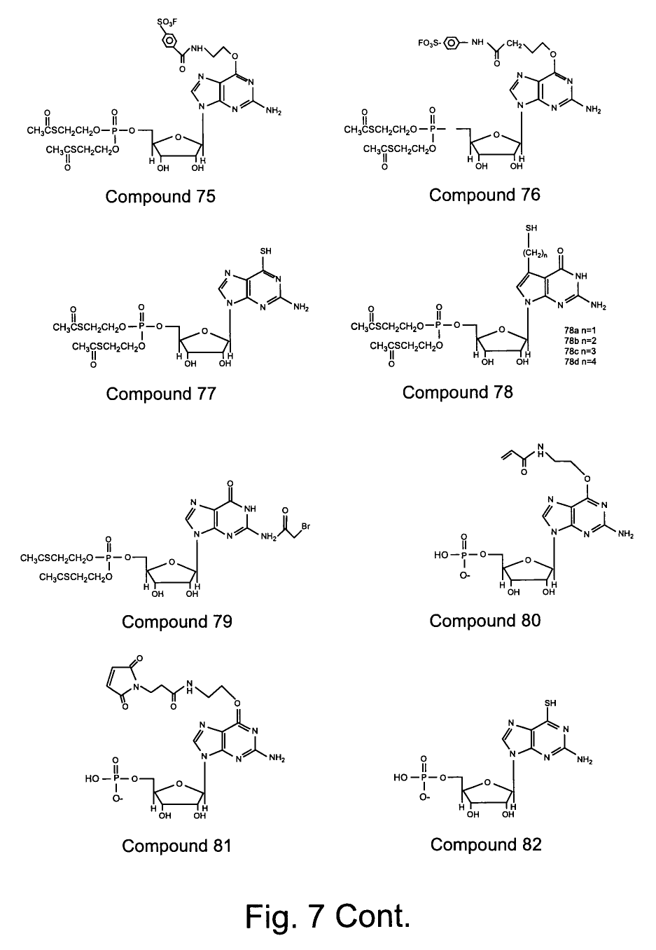

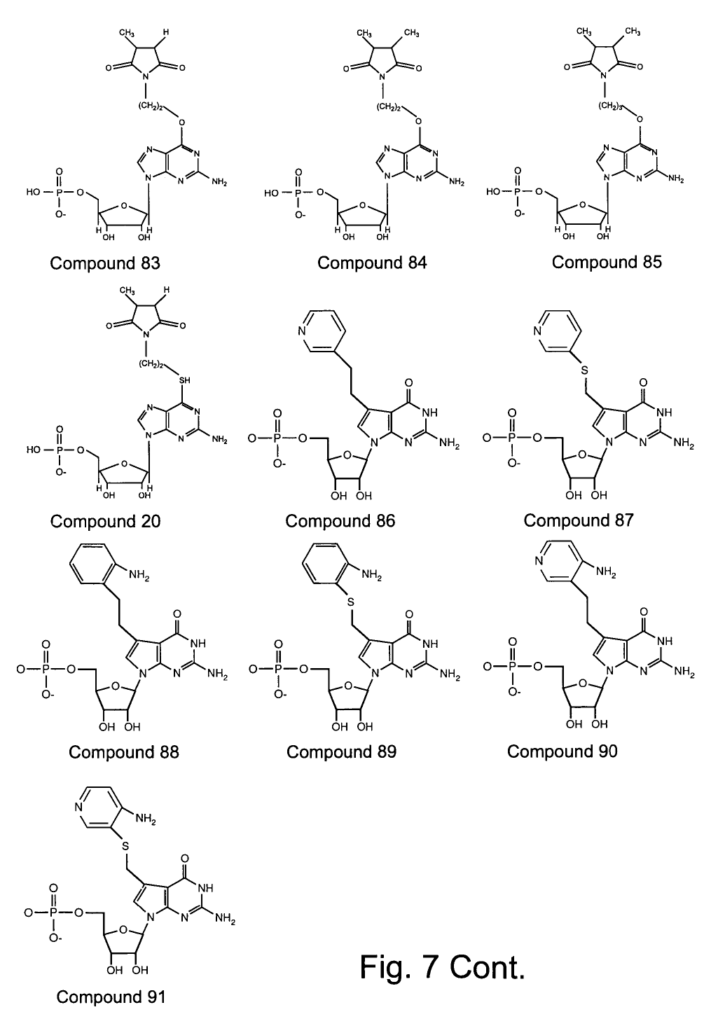

Exemplary modulator structures based upon the GTP scaffolds are provided in FIG. 5. In a similar manner, modulators based upon either a GDP or GMP scaffold are also contemplated (see FIGS. 6 and 7, respectively).

For example, (2-phenyl)ethyl-GDP (compound 60, FIG. 6) is an exemplary orthogonal modulator of the present invention that acts as a selective inhibitor of a mutant GTPase having alanine substitutions at positions corresponding to amino acid residues 19 and 116 of Ras (relative to the wild type GTPase). Similarly, (2-phenyl)ethyl-GTP (compound 52, FIG. 5) is an exemplary orthogonal modulator that acts as an activator of the same GTPase.

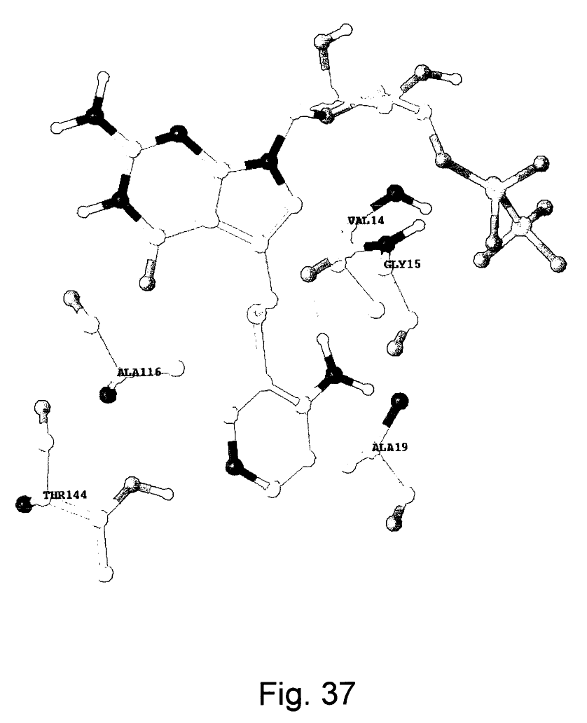

The newly-created cavity of the mutated GTPases, particularly the double mutants, provides additional possibilities for hydrogen bonding with the modulatory compounds of the present invention. An exemplary modulator:GTPase interaction is depicted in FIG. 37. Since a hydrogen bond donor group (such as the hydroxyl group in threonine) is typically found at position 144 in many GTPases, a hydrogen bond acceptor (in this example, a pyridine derivative) is coupled to the purine ring to provide to take advantage of this potential interaction. As modeled in FIG. 37, the new interaction is close to ideal with a distance of 1.86 Ångstroms. Optionally, a sulfanyl group can be included as a linker portion to minimize any potential steric clash, e.g., between the amine hydrogens and methylene hydrogens.

In a further embodiment of the modulators of the present invention, an amine is included at the ortho position of an aromatic ring substituent coupled to the C-7 position (see, for example, compounds 88-91, 94-97 and 100-103). The amine moiety can participate in a hydrogen bond, e.g., with the carbonyl groups of (invariant) amino acids Val14 or Gly15. The aromatic ring can be linked to the purine C-7 position by a number of linker moieties, including, but not limited to, an ethyl linker or a sulfanyl (—CH₂S—) linker.

### Electrophilic Substituents

In one useful embodiment, at least one substituent of the GTPase modulator contains a thiol or an electrophilic moiety capable of forming a covalent bond with an amino acid in the active site of the mutant GTPase. Electrophilic moieties are contemplated for inclusion at any of position 2, 6, 7 or 9 of the purine ring. Exemplary electrophilic moieties that are capable of chemically interacting with the GTPase molecule and forming a covalent bond include, but are not limited to a [(3-maleimido)propylamido]ethyl group, a [(3-methyl)maleimido]ethyl group, a [(3,4-dimethyl)maleimido]ethyl group, a [(3,4-dimethyl)maleimido]propyl group, a (2-N-acrylamido)ethyl group, a (n-N-acrylamido)alkyl group (wherein the alkyl group has between 1-3 carbon atoms), and an alkyl halide. Optionally, the electrophilic substitution is a substituted 3-pyrroline moiety, such as a 2,4-keto(1,5-dimethyl)3-pyrroline (see, e.g., compounds 37, 38, 49, 50, 84 and 85) or a 2,4-keto(1-methyl)3-pyrroline (see, e.g., compounds 36 and 83). Preferably, the pyrroline moiety is coupled (via the N-3 position) to the C-7 position of the guanine ring via an ethyl or propyl linker. However, any of a number of additional reactive groups are known in the art and can be used to form covalent bonds between a small molecule and an amino acid of a protein (see for example Handbook of Fluorescent Probes and Research Products, ninth edition (2003), by Molecular Probes).

### Additional Scaffold Modifications

In addition to the modifications listed herein, alterations at other purine ring positions, and in other components of the scaffold molecules are also considered. For example, alternative five or six-membered sugar ring structures can be coupled to the guanine or otherwise employed as part of the modulator scaffold, as well as the deoxy and dideoxy forms of the sugar rings. The purine scaffold may also be modified, for example with the use of a pyrazolo[3,4-d]pyrimidine scaffold. Other scaffolds that mimic guanosine nucleotides, or scaffolds that retain key interactions established between the GTP/GDP molecules and the protein, are also considered. Modifications at the purine C-2 position are also contemplated, an example of which is shown in FIG. 4, compound 35.

In addition, the phosphate groups optionally further include substituents to “cage” the charged elements or otherwise assist in increasing the cell permeability of the phosphorylated modulator compounds. Caging substituents which can be coupled to the phosphate elements of the modulator include, but are not limited to, —OCH₂OCH₂CH₃, —OCH₂CH₂SCOCH₃, and —O(CH₂)₅SCOCH₃) or other substituents to assist in increasing the cell permeability of the phosphorylated modulator compounds. See, for example, the compounds provided in FIG. 7.

In a further preferred embodiment, derivatized or modified phosphate groups are contemplated for use in the compositions of the present invention. The substitution of an oxygen atom on the terminal phosphate of triphosphate-containing compounds, e.g., with a sulfur atom, is envisioned as a way to prevent the hydrolysis of this terminal phosphate by the mutant enzyme and therefore potentially keep the mutant GTPase in the “on” position for an extended period of time. Additional phosphate modifications are described in Hermanson Bioconjugate Techniques (Elsevier Science/Academic Press, San Diego Calif.).

Methods for the synthesis of the GTPase modulators of the present invention are provided herein. Additional information regarding synthesis of the modulators can be found in, for example, Fessendon and Fessendon, (1982) Organic Chemistry, 2nd Edition, Willard Grant Press, Boston Mass; and Carey & Sundberg, (1990) Advanced Organic Chemistry, 3rd Edition, Parts A and B, Plenum Press, New York. Optionally, the standard chemical reactions described therein are modified to enhance reaction efficiency, yield, and/or convenience.

### Preparation of Modulator Compounds

The present invention also provides methods for the preparation of GTPase modulators, e.g., inhibitors and activators. The methods of the present invention include the steps of a) providing a scaffold structure comprising a purine moiety; b) derivatizing the purine moiety at a first position; and c) optionally derivatizing the purine moiety at a second position. In one embodiment, the derivatizing the purine ring at the first position includes coupling a substituent at the C-6 position of the purine ring; in an alternate embodiment, derivatizing at the first position includes coupling a substituent at N-7. For the ribose-containing modulator compounds, the method further includes the step of ribosylating the derivatized purine moiety at the N-9 position.

The coupled substituent can be either O-linked or N-linked to the purine ring. Optionally, providing the scaffold structure further includes modifying the purine ring at position 7 to contain a carbon atom instead of a nitrogen. Thus, a substituent at position 7 on the purine ring can include an N-linked moiety, or if the purine ring has been altered to contain a carbon at this position, an C-linked derivative. In a preferred C-linked derivative embodiment, coupling at a C-7 position on the purine ring is performed via a Sonogashira coupling reaction, leading to derivatives linked to the purine ring via an ethyl linkage.

Optionally, derivatizing the purine moiety at a second position provides substituents at both the C-6 and N-7 (or C-7) positions.

Preferred scaffold structures for use in the synthesis methods of the present invention are guanine, guanosine and 7-deazaguanosine. Optionally, GTPase modulators based on the guanine scaffold are further derivatized at the N-9 position of the purine ring with, for example, benzyl or (dimethyl)propyl moieties.

### Guanine-Based GTPase Modulators

In one embodiment, the present invention provides GTPase modulators having a guanine cores structure. Typically, the purine ring of the guanine scaffold is modified at one or more of the C-6, N-7 or N-9 positions. Optionally, the nitrogen atom present at position-7 of the purine ring is replaced with a carbon atom, thereby providing modulators having C-linked substituents at position 7. Exemplary GTPase modulators having a guanine-type scaffold are provided, for example, in Table 1.

### Synthesis of C-6-Modified Guanine Derivatives

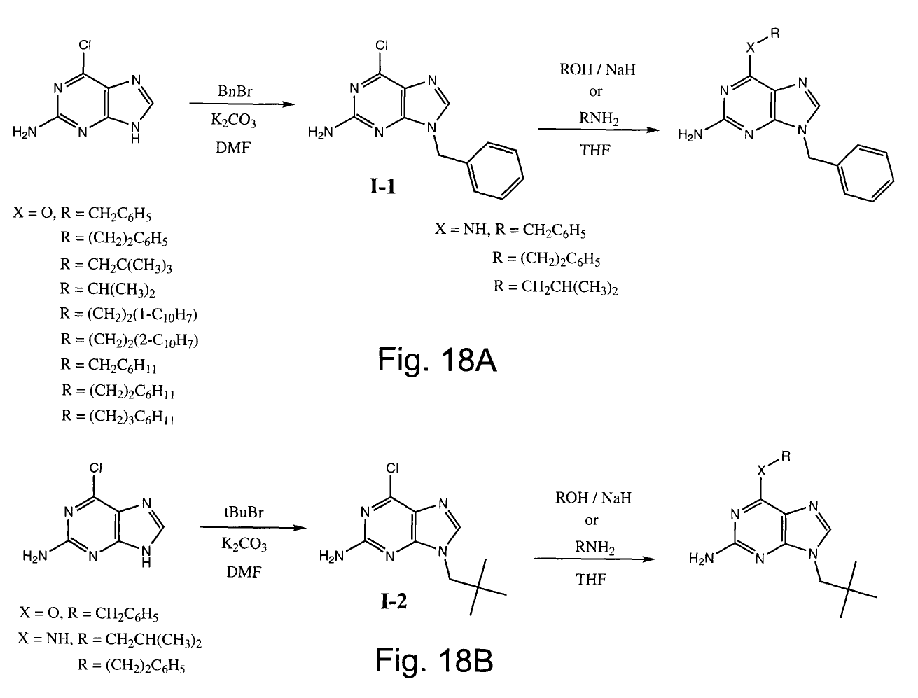

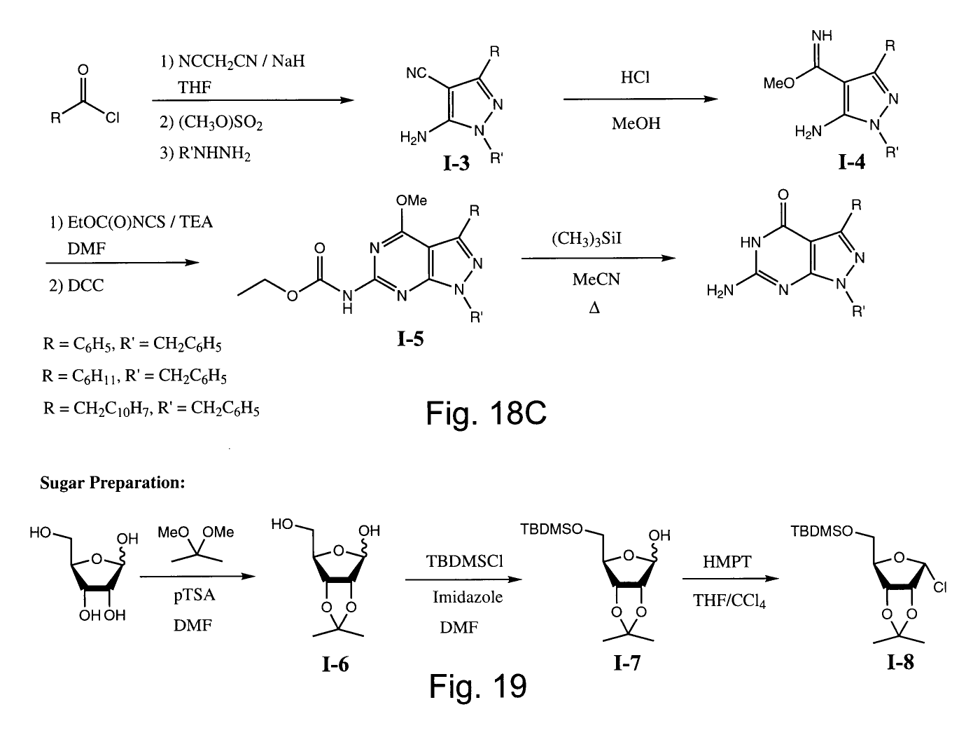

The modulators, e.g., inhibitors and activators, of the present invention include compounds having a purine moiety derivatized at the C-6 position of the purine ring. Optionally, the N-9 position of the purine ring is also derivatized. For the GTPase modulators lacking the ribose moiety at N-9 (e.g., modulators based on a guanine scaffold), one preferred substituent for use at this position is a benzyl group. The corresponding guanine-based modulators can be synthesized using a common synthetic intermediate, 2-amino-9-N-benzyl-6-chloropurine (intermediate I-1), which can be prepared as follows (see also FIG. 18, synthesis scheme A).

### Exemplary Synthesis: Preparation of synthetic intermediate 2-amino-9-N-benzyl-6-chloropurine(I-1)

2-Amino-6-chloropurine (152 mg, 0.896 mmol) and potassium carbonate (184 mg, 1.33 mmol) were suspended in anhydrous DMF (3 mL). Benzyl bromide (101 μL, 0.0849 mmol) was then added, and the resulting reaction mixture was stirred at room temperature for 5 h. Upon completion of the reaction, the mixture was filtered through a cotton plug. The solvent was removed in vacuo and the residue was extracted with EtOAc (3×20 mL)/NaHCO₃. Column chromatography purification (EtOAc/hexanes) afforded 124 mg of the halogenated intermediate 2-amino-9-N-benzyl-6-chloropurine (intermediate I-1, 53% yield) as a white solid. Rf: 0.20 (50% EtOAc/hexanes). ¹H NMR (NMF_{d7}): δ 5.40 (2H, s), 6.98 (2H, s), 7.32-7.42 (5H, m), 8.31 (1H, s); MS (M+H⁺): 260.1.

### Exemplary Synthesis: Preparation of 6-O-alkyl-2-amino-9-N-benzylpurines

The 2-amino-9-N-benzyl-6-chloropurine compound (I-1) can be used for the preparation of a number of C-6 modified modulator compounds. In one embodiment, this intermediate is used to prepare 6-O-alkyl-2-amino-9-N-benzylpurine GTPase modulators as follows.

Sodium hydride (23 mg, 0.96 mmol) was suspended in anhydrous THF (10 mL). The selected alkyl alcohol ROH (0.96 mmol) was then added and the resulting mixture was stirred at room temperature for 30 min to preform the alkoxide. A solution of 2-amino-9-N-benzyl-6-chloropurine (50 mg, 0.19 mmol) in THF (2 mL) was added to the mixture and the reaction was allowed to proceed at 65° C. for 5 h. After removal of the solvents in vacuo, the residue was extracted with EtOAc (3×20 mL)/NaHCO₃. Column chromatography purification (EtOAc/hexanes) afforded the corresponding 6-O-alkylated modulator compound (7-85% yield). Exemplary GTPase modulator compounds prepared by this method include:

2-amino-6-O-benzyl-9-N-benzylpurine (1). (52% yield). Rf: 0.48 (EtOAc). ¹H NMR (CDCl₃): δ 4.63 (2H, d, J=6.0 Hz), 5.18 (2H, s), 5.52 (2H, s), 7.21-7.46 (10H, m), 7.52 (1H, s); MS (M+H⁺): 331.2.

2-amino-6-O-(2′-phenyl)ethyl-9-N-benzylpurine (2). (7% yield). Rf: 0.72 (5% MeOH/CHCl₃). ¹H NMR (CDCl₃): δ 3.09 (2H, t, J=7.2 Hz), 4.59 (2H, t, J=7.2 Hz), 5.24 (2H, s), 6.43 (2H, brs), 7.20-7.35 (10H, m), 7.94 (1H, s); ¹³C NMR (CD₃OD): δ 36.3, 47.7, 68.1, 116.2, 127.8, 128.8, 129.2, 129.8, 130.2, 130.4, 138.5, 141.0, 161.2, 162.3; MS (M+H⁺): 346.2.

2-amino-6-O-(2′,2′-dimethyl)propyl-9-N-benzylpurine (5). (85% yield). Rf: 0.54 (EtOAc). ¹H NMR (CDCl₃): δ 1.07 (9H, s), 4.25 (2H, s), 5.05 (2H, brs), 5.21 (2H, s), 7.12-7.41 (5H, m), 7.52 (1H, s); ¹³C NMR (CDCl₃): δ 27.0, 32.1, 47.1, 76.6, 115.7, 127.9, 128.5, 129.3, 136.4, 139.5, 154.6, 160.0, 162.3; MS (M+H⁺): 312.2.

2-amino-6-O-(2′-methyl)ethyl-9-N-benzylpurine (7). (25% yield). Rf: 0.16 (50% EtOAc/hexanes). ¹H NMR (CDCl₃): δ 1.43 (6H, d, J=6.2 Hz), 4.81 (2H, brs), 5.23 (2H, s), 4.59 (1H, sep, J=6.2 Hz), 7.23-7.34 (5H, m), 7.53 (1H, s); ¹³C NMR (CDCl₃): δ 22.5, 47.1, 70.2, 116.0, 128.0, 128.6, 129.4, 136.3, 139.4, 154.5, 159.9, 161.5; MS (M+H⁺): 284.2.

2-amino-6-O-(2′-naphtyl)methyl-9-N-benzylpurine (9). (36% yield). Rf: 0.77 (50% EtOAc/hexanes). ¹H NMR (CDCl₃): δ 4.94 (2H, brs), 5.20 (2H, s), 5.72 (2H, s), 7.20-7.82 (12H, m), 7.95 (1H, s); MS (M+H⁺): 382.2.

2-amino-6-O-(3′-cyclohexyl)propyl-9-N-benzylpurine (10). (74% yield). Rf: 0.67 (10% MeOH/CHCl₃). ¹H NMR (CDCl₃): δ 0.87-0.92 (2H, m), 1.16-1.37 (6H, m), 1.66-1.73 (5H, m), 1.84 (2H, qu, J=7.2 Hz), 4.46 (2H, t, J=6.7 Hz), 5.00 (2H, brs), 5.21 (2H, s) 7.20-7.31 (5H, m), 7.54 (1H, s); ¹³C NMR (CDCl₃): δ 26.7, 26.8, 27.1, 33.7, 33.9, 37.8, 47.1, 115.8, 127.9, 128.6, 129.3, 136.3, 139.5, 154.5, 160.0, 160.0, 162.0; MS (M+H⁺): 366.3.

2-amino-6-O-(2′-cyclohexyl)ethyl-9-N-benzylpurine (11). (73% yield). Rf: 0.76 (10% MeOH/CHCl₃). ¹H NMR (CDCl₃): δ 0.92-0.98 (2H, m), 1.16-1.25 (3H, m), 1.45-1.60 (1H, m), 1.62-1.79 (7H, m), 4.52 (2H, t, J=6.8 Hz), 4.96 (2H, brs), 5.21 (2H, s), 7.21-7.34 (5H, m), 7.54 (1H, s); ¹³C NMR (CDCl₃): δ 26.6, 27.0, 33.6, 34.7, 36.7, 47.1, 65.5, 115.9, 128.0, 128.6, 129.3, 136.3, 139.5, 154.5, 160.0, 162.0; MS (M+H⁺): 352.3.

2-amino-6-O-methylcyclohexyl-9-N-benzylpurine (14). (73% yield). Rf: 0.74 (EtOAc). ¹H NMR (CDCl₃): δ 1.06-1.29 (6H, m), 1.66-1.91 (7H, m), 4.29 (2H, d, J=5.9 Hz), 4.96 (2H, brs), 5.21 (2H, s), 7.21-7.32 (5H, m), 7.54 (1H, s); MS (M+H⁺): 338.3.

2-amino-6-O-(2′-(2″-naphtyl)ethyl-9-N-benzylpurine (15). (36% yield). Rf: 0.12 (50% EtOAc/hexanes). ¹H NMR (DMF_{d7}): δ 3.33 (2H, t, J=7.2 Hz), 4.76 (2H, t, J=7.2 Hz), 5.34 (2H, s), 6.50 (2H, brs), 7.31-7.92 (12H, m), 8.01 (1H, s); ¹³C NMR (DMF_{d7}): δ 35.6, 46.4, 70.0, 114.8, 125.9, 126.5, 127.9, 128.0, 128.0, 128.1, 128.2, 128.4, 129.2, 132.8, 136.6, 138.1, 140.1, 155.3, 161.0, 161.3; MS (M+H⁺): 396.2.

### Exemplary synthesis methods for preparation of 6-N-alkyl-2-amino-9-N-benzylpurines

### Preparation of 6-N-alkyl-2-amino-9-N-benzylpurines

In addition, GTPase modulators having 6-N-linked alkyl substituents are provided in the present invention. Optionally, these modulators are also prepared using the 6-chloropurine intermediate A as follows.

To a solution of 2-amino-9-N-benzyl-6-chloropurine (I-1, 50 mg, 0.19 mmol) in THF (10 mL) was added the selected alkylamine RNH₂ (4 mmol) and the solution was stirred at 65° C. for 24 h. After removal of the solvents in vacuo, the residue was extracted with EtOAc (3×20 mL)/NaHCO₃. Column chromatography purification (EtOAc/hexanes) afforded the corresponding 6-N-alkylated modulator compound (60-78% yield). Exemplary modulator compounds prepared by this method include:

2-amino-6-N-benzyl-9-N-benzylpurine (3). (60% yield). Rf: 0.13 (EtOAc). ¹H NMR (CDCl₃): δ 4.77 (2H, brs), 5.19 (2H, s), 5.87 (2H, s), 7.17-7.52 (10H, m), 7.79 (2H, s); MS (M+H⁺): 331.2.

2-amino-6-N-(2′-phenyl)ethyl-9-N-benzylpurine (6). (78% yield). Rf: 0.05 (EtOAc). ¹H NMR (CDCl₃): δ 2.88 (2H, t, J=7.2 Hz), 3.81 (2H, brs), 4.98 (2H, s), 5.16 (2H, s), 6.02 (2H, brs), 7.17-7.32 (11H, m); MS (M+H⁺): 345.2.

2-amino-6-N-(2′-methyl)propyl-9-N-benzylpurine (12). (60% yield). Rf: 0.52 (10% MeOH/CHCl₃). ¹H NMR (CDCl₃): δ 0.92 (6H, d, J=6.7 Hz), 2.00 (1H, m), 3.56 (2H, brs), 5.27 (2H, s), 5.86 (2H, brs), 7.08 (1H, brs), 7.26-7.54 (5H, m), 7.81 (1H, s).

### Preparation of synthetic intermediate 2-amino-9-N-(2′,2′-dimethyl)propyl-6-chloropurine (I-2)

In an alternative embodiment, the present invention provides methods for preparing guanine ring scaffolds having a (dimethyl)propyl substituent at the N-9 position. The corresponding modulators can be synthesized using the common intermediate substrate, 2-amino-9-N-(2′,2′-dimethyl)propyl-6-chloropurine (intermediate I-2), which can be prepared as follows (see also FIG. 18, synthesis scheme B).

### Exemplary Synthesis: Preparation of 2-amino-9-N-(2′,2′-dimethyl)propyl-6-chloropurine

2-amino-6-chloropurine (1.01 g, 5.95 mmol) and potassium carbonate (1.4 g, 10.1 mmol) were suspended in anhydrous DMF (5 mL). 1-bromo-2,2-dimethylpropane (tBuCH₂Br, 1.6 mL, 12.7 mmol) was then added, and the resulting reaction mixture was stirred at 100° C. for 40 h. Upon completion of the reaction, the mixture was filtered through a cotton plug. The solvent was removed in vacuo and the residue was extracted with EtOAc (3×20 mL)/NaHCO₃. Column chromatography purification (EtOAc/hexanes) afforded 68 mg of the halogenated intermediate 2-amino-9-N-(2′,2′-dimethyl)propyl-6-chloropurine (I-2, 5% yield) as a white solid. Rf: 0.80 (10% MeOH/CHCl₃). ¹H NMR (CDCl₃): δ 0.93 (9H, s), 3.83 (2H, s), 5.42 (2H, s), 7.70 (1H, s); ¹³C NMR (CDCl₃): 28.0, 33.7, 55.2, 125.1, 143.7, 151.5, 155.1, 159.6; MS (M+H⁺): 240.2.

### Synthetic method for preparation of 6-O-alkyl-2-amino-9-N-(2′,2′-dimethyl)propyl purines

The 9-N-(2′,2′-dimethyl)propyl-containing intermediate I-2 can also be used for the preparation of a number of C-6 modified modulator compounds, e.g., following procedures similar to those described for preparation of the 9-N-benzyl containing C-6 modified modulators. For example, 2-amino-6-O-benzyl-9-N-(2′,2′-dimethyl)propyl purine (compound 8) can be synthesized from this intermediate as follows.

### Exemplary Synthesis: Preparation of 2-amino-6-O-benzyl-9-N-(2′,2′-dimethyl)propyl purine

Sodium hydride (15 mg, 0.62 mmol) was suspended in anhydrous THF (5 mL). Benzyl alcohol (27 μL, 0.067 mmol) was then added and the resulting mixture was stirred at room temperature for 30 min to preform the alkoxide. A solution of 2-amino-9-N-(2′,2′-dimethyl)propyl-6-chloropurine (1-2, 16 mg, 0.07 mmol) in THF (1 mL) was added to the mixture and the reaction was allowed to proceed at 50° C. for 3 h. After removal of the solvents in vacuo, the residue was extracted with EtOAc (3×20 mL)/NaHCO₃. Preparative TLC purification (50% EtOAc/hexanes) afforded 10 mg of 2-amino-6-O-benzyl-9-N-(2′,2′-dimethyl)propyl purine (8) (48% yield). Rf: 0.56 (EtOAc). ¹H NMR (CDCl₃): δ 0.98 (9H, s), 3.85 (2H, s), 4.82 (2H, brs), 5.56 (2H, s), 7.23-7.68 (5H, m), 7.73 (1H, s); MS (M+H⁺): 312.2.

### General synthetic method for preparation of 6-N-alkyl-2-amino-9-N-(2′,2′-dimethyl)propyl purines

In a similar manner, 6-N-alkyl derivatives can be made from intermediate I-2.

### Exemplary Synthesis: for preparation of 6-N-alkyl derivatives

To a solution of 2-amino-9-(2′,2′-dimethyl)propyl-6-chloropurine (I-2, 40 mg, 0.17 mmol) in THF (50 mL) was added the selected alkylamine RNH₂ (1.7 mmol) and the solution was stirred at 65° C. for 18 h. After removal of the solvents in vacuo, the residue was extracted with EtOAc (3×20 mL)/NaHCO₃. Column chromatography purification (MeOH/CHCl₃) afforded the 6-N-alkylated product (60-87% yield). Exemplary modulator compounds prepared by this method include:

2-amino-6-N-(2′-phenyl)ethyl-9-N-(2′,2′-dimethyl)propyl purine (4). (87% yield). Rf: 0.27 (EtOAc). ¹H NMR (CDCl₃): δ 0.98 (9H, s), 2.95 (2H, brs), 3.81 (4H, brs), 4.84 (2H, s), 5.91 (1H, brs), 7.25-7.29 (10H, m), 7.40 (1H, s); MS (M+H⁺): 325.3.

2-amino-6-N-(2′-methyl)propyl-9-N-(2′, 2′-dimethyl)propyl purine (13). (60% yield). Rf: 0.33 (10% MeOR/CHCl₃). ¹H NMR (DMF_{d7}): δ 0.93-0.95 (15H, m), 1.97-2.04 (1H, m), 3.49 (2H, s), 3.85 (2H, s), 5.75 (2H, brs), 6.99 (1H, brs), 7.69 (1H, s); MS (M+H⁺): 277.3.

### Synthesis of position 7-modified guanine derivatives

GTPase modulators (inhibitors, activators) based upon pyrazolo-pyrimidine scaffolds having substituents corresponding to position 7 of the guanine scaffold (position 3 of the pyrazolo-pyrimidine ring structure) are also provided in the present invention. In one embodiment, these modulators are prepared through various intermediate products as shown in FIG. 18, synthetic scheme 3, and as follows.

### Exemplary Synthesis: Preparation of 1-alkyl-3-alkyl-5-amino-4-cyanopyrazoles

Malononitrile (30 mmol) was slowly added to a suspension of sodium hydride (60 mmol) in THF (50 mL). Following the addition of the selected acyl chloride RCOCl (30 mmol), the resulting exothermic reaction was complete after stirring for 1 h. Dimethyl sulfate (40 mmol) was then added and the reaction mixture was stirred at reflux for 5 h and at 25° C. for a further 48 h. After the additions of triethylamine (100 mmol) and the selected alkylhydrazine RNHNH₂ (30 mmol), the reaction mixture was refluxed for another 5 h. After removal of the solvents in vacuo, extraction of the residue was carried out using aqNaHCO₃ (20 mL), brine (20 mL) and EtOAc (3×20 mL). The desired compound was purified by column chromatography (EtOAc/hexanes) and recrystallized from MeOH/water to yield various 1-3 intermediates (6-28% yield). Exemplary intermediate products prepared by this method include:

5-amino-1-benzyl-4-cyano-3-cyclohexylpyrazole (I-3a): 6% yield MS (M+H⁺): 281.2.

5-amino-1-benzyl-4-cyano-3-phenylpyrazole (I-3b): 28% yield, MS (M+H⁺): 275.2.

5-amino-1-benzyl-4-cyano-3-(1′-naphtyl)methylpyrazole (I-3c): 17% yield, MS (M+H⁺): 339.2.

### Exemplary Synthesis: Preparation of 1-alkyl-3-alkyl-4-methoxy-6-(methoxycarbonyl)aminopyrazolo[3,4-d]pyrimidines

The intermediate compounds designated I-3a, I-3b and I-3c (0.70 mmol) were dissolved in MeOH (5 mL) and HCl gas was flowed above the solution for 30 min at 0° C. The resulting solution was then stirred for 18 h at 0° C. After flushing with N₂, the reaction was freeze-dried and the corresponding intermediates I-4 were stored over KOH in vacuo before being used directly in the cyclization step. Crude intermediate I-4 was dissolved in anhydrous DMF (4 mL), and ethoxycarbonyl isothiocyanate (0.70 mmol) was added to the solution. After 1 h of stirring, triethylamine (1.4 mmol) was added to the reaction and stirring was applied for another hour. Dicyclohexylcarbodiimide (0.90 mmol) was then added to the reaction and the resulting solution was stirred for 18 h. After removal of the solvents in vacuo, the residue was purified by column chromatography (EtOAc/hexanes) to yield the corresponding 1-5 intermediates (17-48% yield).

1-benzyl-3-cyclohexyl-4-methoxy-6-(methoxycarbonyl)aminopyrazolo[3,4-d]pyrimidine (I-5a): 41% yield,_MS (M+H⁺): 410.3.

1-benzyl-4-methoxy-6-(methoxycarbonyl)amino-3-phenylpyrazolo[3,4-d]pyrimidine (I-5b): 17% yield, MS (M+H⁺): 404.2.

1-benzyl-4-methoxy-6-(methoxycarbonyl)amino-3-(1′-naphtyl)methylpyrazolo[3,4-d]pyrimidine (I-5c): 48% yield,_{—MS (M+H}⁺): 468.3.

### Exemplary Synthesis: Preparation of 1-alkyl-3-alkyl-6-amino-4-oxopyrazolo[3,4-d]pyrimidines

Iodotrimethylsilane (1 mL) was added to a solution of compounds I-5 (0.07 mmol) in anhydrous MeCN (5 mL). The resulting solution was refluxed for 5 h, and then stirred at 25° C. for 18 h. The reaction was evaporated to dryness in vacuo, and extracted into EtOAc (3×20 mL). The combined organic layers were treated with MgSO₄, dried in vacuo, and the desired products 16-18 were purified by preparative TLC (70% EtOAc/hexanes) in 24-58% yield.

1-benzyl-6-amino-4-oxo-3-phenylpyrazolo[3,4-d]pyrimidine (16): 58% yield, Rf=0.27 (50% EtOAc/hexanes), ¹H NMR (DMSO-d₆): δ 5.35 (2H, s), 6.71 (2H, brs), 7.22-7.42 (8H, m), 8.32 (2H, d, J=8.0 Hz), 10.69 (1H, s); ¹³C NMR (DMSO-d₆): 49.5, 127.2, 127.4, 127.5, 128.1, 128.5, 137.3, 154.7, 156.4, 158.3; MS (M+H⁺): 318.2.

1-benzyl-6-amino-4-oxo-3-(1′-naphtyl)methylpyrazolo[3,4-d]pyrimidine (17): 55% yield, Rf=0.19 (50% EtOAc/hexanes). ¹H NMR (DMSO-d₆): δ 4.53 (2H, s), 5.21 (2H, s), 6.66 (2H, brs), 7.10-8.32 (12H, m), 10.53 (1H, s); ¹³C NMR (DMSO-d₆): δ 30.8, 49.1, 97.6, 124.2, 125.5, 125.8, 126.7, 126.9, 127.0, 127.2, 128.3, 128.4, 128.4, 131.5, 133.3, 135.3, 137.5, 146.8, 154.9, 155.3, 158.3; MS (M+H⁺): 382.2.

1-benzyl-3-cyclohexyl-6-amino-4-oxopyrazolo[3,4-d]pyrimidine (18): 24% yield, Rf=0.26 (50% EtOAc/hexanes). ¹H NMR (DMSO-d₆): δ 1.17-1.32 (3H, m), 1.6-1.99 (7H, m), 2.80 (1H, t, J=4.0 Hz), 5.21 (2H, s), 6.59 (2H, brs), 7.14-7.33 (5H, m), 10.47 (1H, s); ¹³C NMR (DMSO-d₆): δ 25.6, 25.9, 30.9, 37.4, 49.0, 96.4, 127.0, 127.2, 128.4, 137.7, 153.4, 154.7, 155.5, 158.0; MS (M+H⁺): 324.2.

### Guanosine-Based Modulators

The present invention also provides GTPase modulators having a guanosine core structure. Typically, the purine ring of the guanine scaffold is modified at one or more of the C-6 or N-7 positions. Optionally, the nitrogen atom present at position-7 of the purine ring is replaced with a carbon atom, providing modulators having C-linked substituents at position 7. Optionally, the guanosine ring structure is structurally altered at positions 7 and 8 to form a corresponding pyrazolo-pyrimidine structure. Exemplary GTPase modulators having a guanosine-type scaffold (including the phosphorylated versions) are provided, for example, in Tables 2-5.

### Synthesis of C(7)-Modified Guanosine Derivatives

### Preparation of the Functionalized Sugar

For the synthesis of guanosine and phosphorylated guanosine GTPase modulators of the present invention, a functionalized ribose sugar is typically employed, and can be prepared as follows (see also FIG. 19).

### Exemplary Synthesis: Preparation of Functionalized Ribose

To a suspension of D-ribose (20.1 g, 130 mmol) in anhydrous DMF (120 mL) under N₂ was added 2,2-dimethoxypropane (120 mL, 974 mmol) and a catalytic amount of p-toluene sulfonic acid (pTSA, 200 mg). The reaction was stirred at room temperature for 2 hours and then warmed up to 50° C. for 1 h to drive it to completion. After removal of the solvent in vacuo, the residue was purified by column chromatography (7% MeOH/CHCl₃) to yield 2,3-O-(1-methylethylidene)-D-ribose (I-6) in 51% yield as a mixture of alpha and beta anomers, both suitable for use in the synthesis reactions of the present invention. Rf=0.55 (α isomer) and 0.39 (β isomer) (10% MeOH/CHCl₃).

To a solution of 2,3-O-(1-methylethylidene)-D-ribose (I-6, 10.8 g, 56.9 mmol) in anhydrous DMF (30 mL) was added imidazole (6.9 g, 102 mmol) followed by t-butyldimethylsilyl chloride (TBDMSCI, 10.3 g, 68.25 mmol). The reaction solution was stirred at room temperature for 3 hours. Upon completion, the solvent was removed in vacuo and the residue was slurried in CHCl₃ (100 mL) and washed with 1M HCl (2×100 mL). The organic layer was dried over MgSO₄, and purified by column chromatography (3:2 hexane:Et₂O) to give 5-O-(1,1-dimethylethyl)-2,3-O-(1-methylethylidene)-D-ribose (I-7) in 65% yield. Rf=0.80 (3% MeOH/CHCl₃). MS (M+H⁺): 287.2 (—H₂O).

The 5-O-[(1,1-dimethylethyl)-2,3-O-(1-methylethylidene)-D-ribose (1-7, 4.02 g, 13.9 mmol) was dissolved in anhydrous THF (55 mL) and anhydrous CCl₄ (1.98 mL, 21.5 mmol). This solution was cooled to −78° C. before adding HMPT (3.12 mL, 18 mmol). The reaction was allowed to proceed for a total of 2 hours with periodic warming to −45° C. to prevent gelling. For use in the synthesis of the GTPase modulators of the present invention, the functionalized ribose solution resulting from this Appel chlorination (I-8) was typically warmed to room temperature and directly cannulated into the ribosylation reaction mixture (as described below).

### Preparation and Activation of the Purine Ring Structure

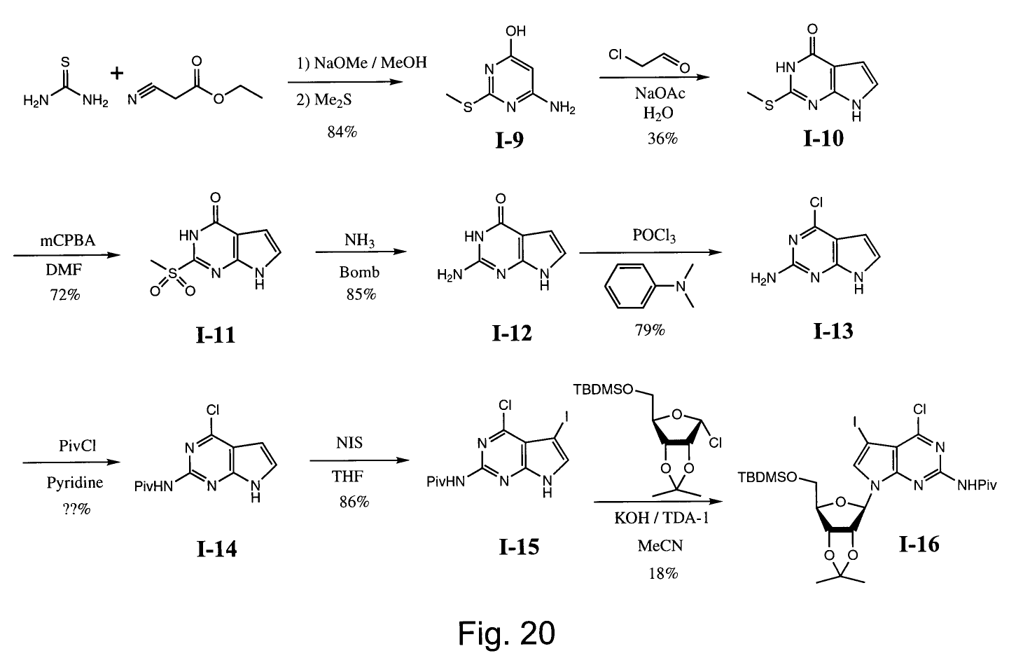

The derivatized purine ring moiety of the guanosine-based modulators of the present invention can be prepared via various intermediate products as follows (FIG. 20).

### Exemplary Synthesis: Preparation of 2-methylthio-4-amino-6-pyrimidone intermediates

The first intermediate in the preparation of the purine ring structure, 2-methylthio-4-amino-6-pyrimidone (intermediate I-9), was prepared as follows. To a solution of sodium (31.6 g, 1.37 mol) in methanol (1 L) was added ethyl cyanoacetate (133 mL, 1.24 mol) and thiourea (102.6 g, 1.35 mol). After refluxing for 2 hours, the heat source was removed and water (240 mL) was added to the yellow suspension. Dimethyl sulfate (116 mL) was then added at a rate to keep the reaction at a gentle reflux. The reaction was then refluxed for an additional 20 minutes before cooling to 4° C. The resultant precipitate was filtered off to yield 110.76 g of pure 2-methylthio-4-amino-6-pyrimidone (1-9, 57% yield). A second crop of crystals could be recovered by solvent removal in vacuo to 500 mL, while this increased the yield to 84% this material proved unsuitable for the subsequent reactions. Rf=0.31 (5% MeOH/CHCl₃). ¹H NMR (DMSO-d₆): δ 2.42 (3H, s), 4.90 (1H, s), 6.45 (2H, brs), 11.45 (1H, brs); ¹³C NMR (DMSO-d₆): 12.6, 81.2, 162.7, 163.5, 164.2; MS (M+H⁺): 158.1.

Sodium acetate (110 g, 1.34 mol) and 2-methylthio-4-amino-6-pyrimidone (I-9,115.5 g, 0.735 mol) were combined in hot water (1.65 L, 80° C.) to yield a milky white suspension. Chloroacetaldehyde (116 mL, 50% in water, 0.913 mol) was then added and the reaction was stirred at 80° C. for 20 minutes. Upon cooling to room temperature, the brown solid was filtered off and washed with water and then acetone to yield 45.3 g of off-white solid 2-methylthio-4-pyrrolo[2,3d]pyrimidone (intermediate I-10, 36% yield). Rf=0.46 (5% MeOH/CHCl₃). ¹H NMR (DMSO-d₆): δ 2.52 (3H, s), 6.46 (1H, d, J=8.0 Hz), 7.91 (1H, t, J=8.0 Hz), 11.86 (1H, brs), 12.15 (1H, brs); ¹³C NMR (DMSO-d₆): 12.8, 102.0, 104.2, 119.3, 148.3, 154.2, 158.9; MS (M+H⁺): 182.1.

To a cooled suspension of I-10 (229.8 g, 1.27 mol) in DMF (4.22 L) was added m-chloroperoxybenzoic acid (mCPBA, 730 g, 3.25 mol) portion-wise over a period of 1 hour. This deep red solution was stirred at room temperature for 13 h. Solvent from the resulting orange suspension was removed in vacuo almost to dryness. The residue was slurried into 1 M HCl (2 L) and the precipitate collected by filtration. Further washes with ethyl ether (5×500 mL) to remove benzoic acid yielded 194.53 g of pure 2-sulfone-4-pyrrolo[2,3d]pyrimidone (intermediate I-11, 72% yield) as an orange solid. Rf=0.27 (5% MeOH/CHCl₃). ¹H NMR (DMSO-d₆): δ 3.39 (3H, s), 6.61 (1H, d, J=8.0 Hz), 7.42 (1H, s), 12.49 (1H, brs), 12.98 (1H, brs); MS (M+H⁺): 214.1.

For the preparation of 2-amino-4-pyrrolo[2,3d]pyrimidone (intermediate I-12, deazaguanine), a portion of intermediate I-11 (20 g, 94 mmol) was placed in a dry bomb under vacuum and the apparatus was cooled to −78° C. The bomb was backfilled with nitrogen before condensing ammonia (200 mL) directly in the steel bomb. The reaction was then stirred at 100° C. for 48 hours. The product was washed with saturated sodium bicarbonate and dried to yield 11.9 g of 2-amino-4-pyrrolo[2,3d]pyrimidone (I-12, 7-deazaguanine, 85% yield).

For the next step in the synthesis, 2-amino-4-chloro-pyrrolo[2,3d]pyrimidine (intermediate I-13) was prepared as follows. 2-Amino-4-pyrrolo[2,3d]pyrimidone (intermediate I-12, 20.4 g, 136 mmol) was refluxed in a mixture of phosphorus oxychloride (300 mL, 3.21 mol) and N,N-dimethylaniline (18 mL, 142 mmol) for several hours. The reaction was cooled down to room temperature before removing the solvent in vacuo. The viscous brown residue was cooled in an ice bath before being quenched by water (250 mL) and filtered. The filtrate was treated with ammonium hydroxide until the pH reached 4. The resulting precipitate was filtered off and washed with water to yield 18.2 g of semi-pure 2-amino-4-chloro-pyrrolo[2,3d]pyrimidine (intermediate 1-13, 79% yield). Rf=0.49 (5% MeOH/CHCl₃). ¹³C NMR (DMSO-d₆): 98.6, 108.5, 123.1, 150.8, 154.5, 159.3; MS (M+H⁺): 169.1.

To a stirring solution of 2-amino-4-chloro-pyrrolo[2,3-d]pyrimidine (I-13, 100 mg, 0.67 mmol) in anhydrous pyridine (2 mL) was added pivaloyl chloride (90 μL, 0.73 mmol). The reaction was stirred at room temperature under nitrogen for 1 hour. The solvent was then removed in vacuo and the residue dissolved in ethyl acetate (30 mL) and washed with 0.1 M HCl (3×50 mL). The organic layer was dried over MgSO₄ and evaporated. The crude material was purified by silica chromatography (30% ethyl acetate/hexane) to yield 97 mg of I-14 as a white crystalline solid (62% yield). MS (M+H⁺): 253.2.

The intermediate I-14 (6.0 g, 24 mmol) was dissolved in anhydrous THF (100 mL) under nitrogen. After the addition of N-iodosuccinimide (NIS, 6.4 g, 28 mmol) the reaction was stirred at room temperature for 1 hour. The solvent was then removed in vacuo and the residue dissolved in ethyl acetate (100 mL) and washed with 1 M sodium thiosulfate (3×100 mL). Column chromatography purification (2% MeOH, isocratic) yielded 7.7 g (86% yield) of 4-chloro-5-iodo-2-trimethylacetamido-pyrrolo[2,3d]pyrimidine (the functionalized purine ring, intermediate I-15). MS (M+H⁺): 379.0.

### Formation of the functionalized Guanosine Scaffold

This di-halogenated purine ring can then be coupled with the functionalized ribose moiety as described herein, to form a functionalized guanosine scaffold (e.g., intermediate I-16: 4-chloro-7-{5′-O-[(1,1-dimethylethyl)dimethylsilyl]-2′,3′-O-(1-methylethylidene)-β-D-ribofuranosyl}-5-iodo-2-trimethylacetamido-pyrrolo[2,3d]pyrimidine), from which the various phosphorylated and nonphosphorylated guanosine-based GTPase modulators of the present invention can be prepared.

### Exemplary Synthesis: Preparation of a Functionalized Guanosine Scaffold

Coupling of the purine ring and ribose moiety to form a functionalized guanosine scaffold for derivatization was performed as follows. Powdered KOH (1.03 g, 25 mmol) and tris[2-(2-methoxyethoxyl)-ethyl]amine (TDA-1, 53 μL, 0.16 mmol) were stirred in anhydrous MeCN (50 mL) for 10 minutes before adding 4-chloro-5-iodo-2-trimethylacetamido-pyrrolo[2,3d]pyrimidine (I-15; 3.059 g, 8 mmol). The reaction was stirred for an additional 10 minutes before adding freshly prepared 5-O-[(1,1-dimethylethyl)-2′,3′-O-(1-methylethylidene)-α-D-ribofuranosyl chloride (I-8, 2.61 g, 8 mmol, calculated on the basis of 100% yield of the Appel chlorination). This reaction was stirred at room temperature for 20-30 hours. Insoluble material was filtered off and the filtrate evaporated in vacuo. The residue was taken up in CH₃Cl₃ (100 mL), washed with H₂O (100 mL), and the organic layer dried by MgSO₄ and evaporated. The residue was chromatographed (8% ethyl acetate/hexane) to yield 2.335 g of yellow amorphous solid containing the desired product mixed with the starting sugar. A further optional purification by preparative HPLC yielded 0.97 g of the functionalized guanosine scaffold (4-chloro-7-{5′-O-[(1,1-dimethylethyl)dimethylsilyl]-2′,3′-O-(1-methylethylidene)-β-D-ribofuranosyl}-5-iodo-2-trimethylacetamido-pyrrolo[2,3d]pyrimidine (intermediate I-16) as a white fluffy powder (18% yield). MS (M+H⁺): 665.2.

### Synthesis of C(7)-Modified Guanosine Derivatives

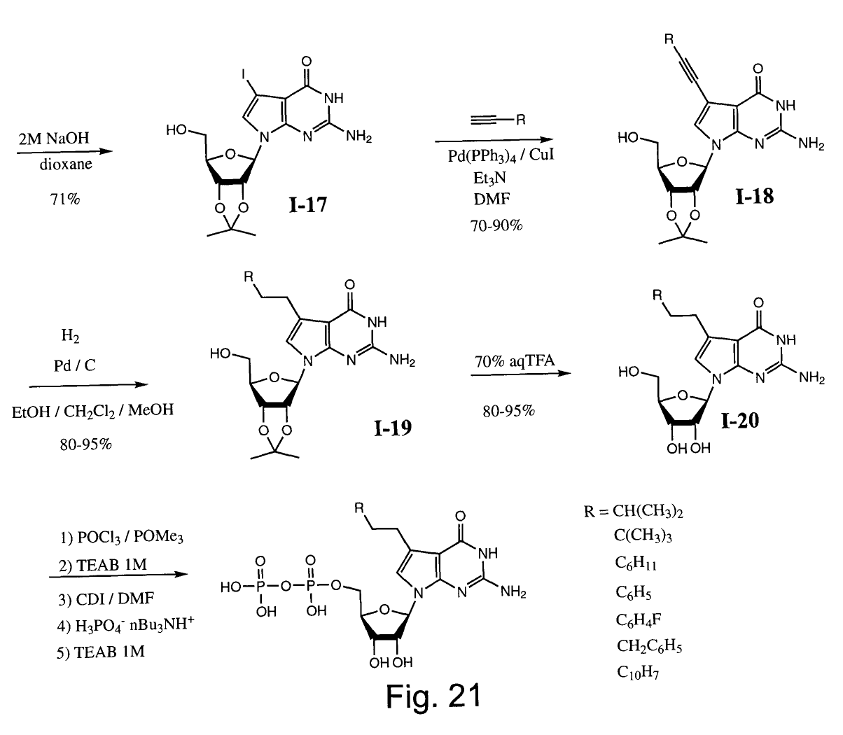

The C(7)-modified guanosine derivatives can be prepared, for example, from a functionalized guanosine scaffold as follows (FIG. 21). Typically, the functionalization at the C(6) position is neutralized to form a ketone moiety prior to coupling a substituent at C(7).

### Exemplary Synthesis: Preparation of C(7)-Modified Guanosine Derivatives

The functionalized guanosine scaffold (I-16, 130 mg, 0.20 mmol) was dissolved in a mixture of 2N NaOH (5 mL) and dioxane (5 mL). The reaction was stirred at reflux for 4 hours before being cooled down, neutralized with AcOH. After removal of the solvents in vacuo, the residue was dissolved in CHCl₃ (75 mL) and washed with H₂O (75 mL). Column chromatography purification (3% MeOH/CHCl₃) afforded 62 mg of 2-amino-5-iodo-7-[2′,3′-O-(1-methylethylidene)-β-D-ribofuranosyl]-4-pyrrolo[2,3d]pyrimidone (I-17, 71% yield). MS (M+H⁺): 449.1.

The C(7) substituent is then coupled to the mono-halogen functionalized intermediate. In a preferred embodiment, the C(7)-halogenated guanosine derivative and a selected R-group having an alkyne linker moiety (R—CCH) are coupled via a Sonogashira coupling reaction (see, for example Sonogashira et al. (1975) Tetrahedron Lett. 4467). 2-Amino-5-iodo-7-[2′,3′-O-(1-methylethylidene)-β-D-ribofuranosyl]-4-pyrrolo[2,3d]pyrimidone (I-17, 0.40 mmol), Cu(I)I (0.05 mmol) and Pd(PPh₃)₄ (0.04 mmol) were combined in a flame-dried 10 mL round-bottom flask before evacuating air and backfilling with N₂. To this mixture was added anhydrous DMF (4 mL), the alkyne (0.48 mmol) and Et₃N (0.80 mmol). The reaction was stirred at room temperature for 4 hours under an N₂ atmosphere. The solvents were removed in vacuo, and the residue dissolved in CHCl₃ (50 mL) and washed with H₂O (2×50 mL). Column chromatography (3% MeOH) yielded the desired alkyne-derivatized guanosine compounds 1-18 (e.g., guanosine-CC—R group) in 72-98% yield. Exemplary modulator intermediates prepared by this method include:

7-deaza-7-[(3″,3″-dimethyl)1″-butynyl]-2′,3′-O-(1-methylethylidene)guanosine (I-18a). (73% yield).

7-deaza-7-[(3″-methyl)1″-butynyl]-2′,3′-O-(1-methylethylidene)guanosine (I-18b). (72% yield).

7-deaza-7-[(2″-cyclohexyl)1″-ethynyl]-2′,3′-O-(1-methylethylidene)guanosine (I-18c). (74% yield).

7-deaza-7-[(2″-phenyl)1″-ethynyl]-2′,3′-O-(1-methylethylidene)guanosine (I-18d). (89% yield).

7-deaza-7-{[2″-(4-fluoro)phenyl]1″-ethynyl}-2′,3′-O-(1-methylethylidene)guanosine (I-18e). (87% yield).

7-deaza-7-[(3″-phenyl)1″-propynyl]-2′,3′-O-(1-methylethylidene)guanosine (I-18f). (73% yield).

7-deaza-7-{[2″-(1-naphtyl)]1″-ethynyl}-2′,3′-O-(1-methylethylidene)guanosine (I-18g). (98% yield).

To reduce the alkyne moiety to an alkane linker group, a palladium catalyst was prepared. Pd/C (125 mg) was added to 10 mL of anhydrous ethyl acetate. Under an H₂ atmosphere the catalyst was pre-reduced for 1 hour. To this was added the alkyne-derivatized guanosine compound (I-18, 0.25 mmol) dissolved in anhydrous CH₂Cl₂ (10 mL) and anhydrous MeOH (1.5 mL). The reaction was stirred at room temperature with H₂ bubbling into the solution until full reduction was achieved (approximately 1 to 8 hours). The catalyst was then filtered off and the solvents removed in vacuo to yield the further guanosine intermediate having an ethyl-linked R-group (I-19, 44-97% yield). Exemplary intermediate I-19 compounds include:

7-deaza-7-(3″,3″-dimethyl)butyl-2′,3′-O-(1-methylethylidene)guanosine (I-19a). (89% yield).

7-deaza-7-(3″-methyl)butyl-2′,3′-O-(1-methylethylidene)guanosine (I-19b). (44% yield).

7-deaza-7-(2″-cyclohexyl)ethyl-2′,3′-O-(1-methylethylidene)guanosine (I-19c). (80% yield).

7-deaza-7-(2″-phenyl)ethyl-2′,3′-O-(1-methylethylidene)guanosine (I-19d). (77% yield).

7-deaza-7-[2″-(4-fluoro)phenyl]ethyl-2′,3′-O-(1-methylethylidene)guanosine (I-19e). (90% yield).

7-deaza-7-(2″-phenyl)propyl-2′,3′-O-(1-methylethylidene)guanosine (I-19f). (97% yield).

7-deaza-7-(2″-(1-naphtyl))ethyl-2′,3′-O-(1-methylethylidene)guanosine (I-19g). (85% yield).

### Exemplary Synthesis: Isopropylidene Deprotection

As a final step in the preparation of the nonphosphorylated guanosine derivatives of the present invention, the isopropylidene protecting group on the ribose moiety is removed.

The compounds (0.20 mmol) were dissolved in 70% aqueous TFA and stirred at room temperature for 1 hour. The solvents were removed in vacuo and the residue was dissolved in MeOH (3×20 mL) and purified by preparative TLC (20% MeOH/CHCl₃) to yield the desired C(7)-modified guanosine-based modulator compounds (65-96% yield). Exemplary GTPase modulators based upon a nonphosphorylated guanosine scaffold as prepare by this method include:

7-deaza-7-(3″-methyl)butyl guanosine (65% yield).

7-deaza-7-(3″,3″-dimethyl)butyl guanosine (96% yield).

7-deaza-7-(2″-cyclohexyl)ethyl guanosine (63% yield).

7-deaza-7-(2″-phenyl)ethyl guanosine (94% yield).

7-deaza-7-[2″-(4-fluoro)phenyl]ethyl guanosine (71% yield).

7-deaza-7-(2″-phenyl)propyl guanosine (68% yield).

7-deaza-7-(2″-(1-naphtyl)ethyl guanosine (85% yield).

The di-phosphorylated modulator structures corresponding to these guanosine modulators are provided in FIG. 6, compounds 57-63.

### General Synthetic Method for (6)-N-Modified Guanosines

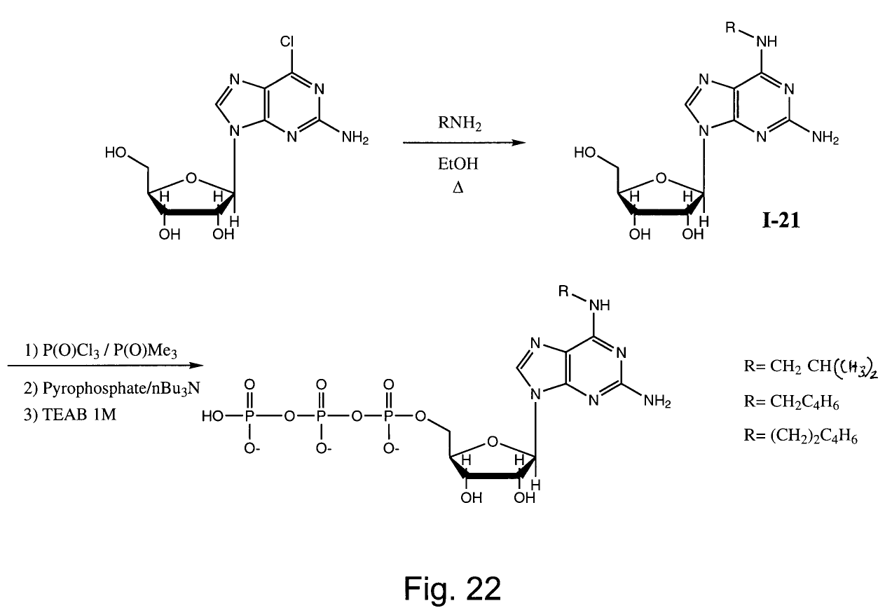

As noted previously, (6)-N-modified guanosine compounds are also contemplated as GTPase modulators in the present invention, and can be prepared as follows (also see FIG. 22).

To a solution of 2-amino-6-chloropurine riboside (100 mg, 0.33 mmol) in anhydrous EtOH (10 mL) was added a primary amine (3.33 mmol). The resulting solution was refluxed overnight. The solvent was removed in vacuo and the residue was purified by column chromatography (10% MeOH/CHCl₃) to afford the N-linked (C-6)-modified compounds of the present invention (I-21, 76-100% yields). Exemplary (C-6)-modified modulators prepared by this method include, but are not limited to:

6-N-benzyl guanosine: 100% yield. Rf: 0.40 (10% MeOHI/CHCl₃). ¹³C NMR (CD₃OD): δ 61.3, 62.8, 71.1, 72.0, 72.2, 73.9, 87.2, 90.3, 114.7, 127.1, 127.4, 127.6, 127.8, 128.5, 128.7, 139.5, 155.6, 160.5; MS (M+H⁺): 373.2.

6-N-(2′-methylpropyl)guanosine: 90% yield. Rf: 0.38 (10% MeOH/CHCl₃). ¹H NMR (CD₃OD): δ 0.98 (6H, brs), 1.93 (1H, brs), 3.36 (2H, s), 3.75 (1H, d, J=12 Hz), 3.9 (1H, d, J=12 Hz), 4.18 (1H, s), 4.33 (1H, s), 5.82 (1H, brs), 7.86 (1H, s); ¹³C NMR (DMF): δ14.1, 20.4, 32.4, 61.6, 63.3, 71.0, 72.5, 74.4, 87.5, 89.6, 115.3, 137.5, 151.0, 156.4, 161.0; MS (M+H⁺): 338.6.

6-N-(2′-ethylphenyl)guanosine: 76% yield. Rf: 0.48 (10% MeOH/CHCl₃). ¹H NMR (CD₃OD): δ 2.87 (2H, t, J=7.4 Hz), 3.70 (3H, dd, J₁=2.2 Hz, J₂=12.6 Hz), 3.86 (1H, dd, J₁=2.2 Hz, J₂=12.6 Hz), 4.15 (1H, d, J=2.0 Hz), 4.31 (1H, dd, J₁=1.9 Hz, J₂=5.0 Hz), 4.77 (1H, dd, J₁=5.2 Hz, J₂=6.6 Hz), 5.79 (1H, d, J=6.8 Hz), 7.09-7.23 (5H, m), 7.78 (1H, s); ¹³C NMR (CD₃OD): 36.8, 63.9, 73.1, 74.9, 88.3, 91.3, 115.7, 127.4, 129.5, 130.0, 140.7, 156.6, 161.5; MS (M+H⁺): 386.7.

### Exemplary Synthesis: Preparation of (6)-O-Modified Guanosines

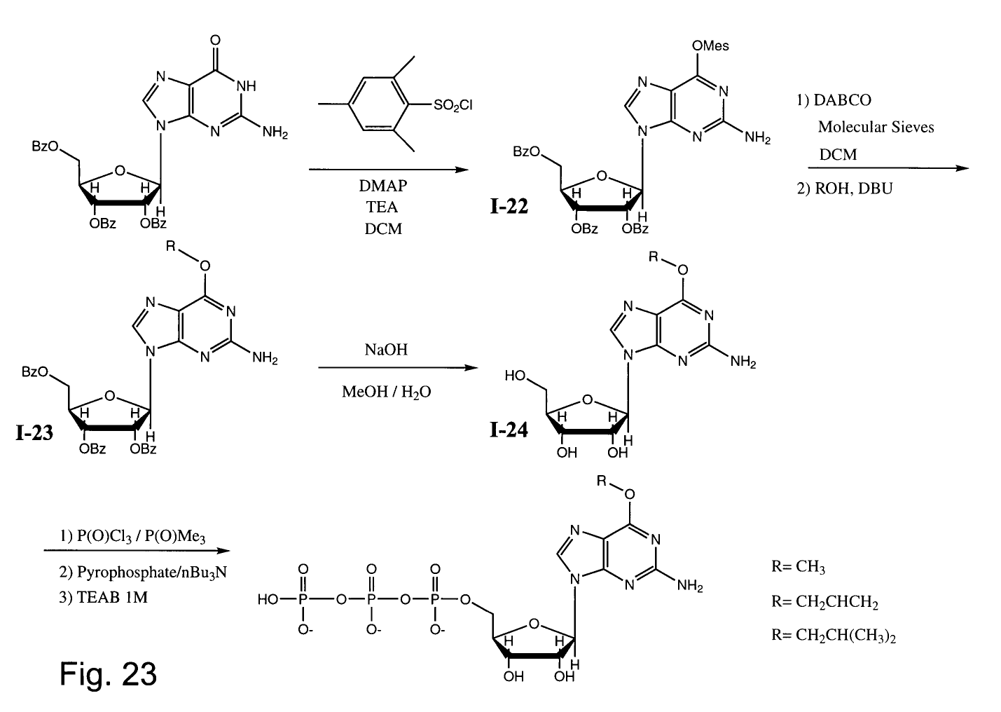

The GTPase modulators of the present invention having structures based upon a (6)-O-modified guanosine scaffold can be prepared as follows (also see FIG. 23).

2′,3′,5′-Tri-O-benzoyl-guanosine (5.95 g, 10 mmol) and 4-dimethylaminopyridine (DMAP, 122 mg, 1 mmol) was suspended in CH₂Cl₂. After the addition of TEA (4.15 mL, 30 mmol), the reaction mixture was chilled to 0° C. for 15 min. The addition of (2,4,6-trimethylphenyl)sulfonyl chloride (4.66 g, 21.3 mmol) was followed by stirring at 0° C. for 30 min. after which the reaction mixture was allowed to warm up to room temperature over 4 h. Silica gel (50 g) was added to the pink solution and the solvents were removed in vacuo. Column chromatography (EtOAc/hexanes) afforded 7.65 g of the 6-Mes derivative (I-22, 99% yield) as a pale yellow solid. Rf: 0.42 (50% EtOAc/hexanes). ¹H NMR (CD₃OD): δ 2.30 (3H, s), 2.74 (6H, s), 4.66 (1H, dd, J₁=4.5 Hz, J₂=12.0 Hz), 4.79 (1H, q, J=4.6 Hz), 4.87 (1H, dd, J₁=3.8 Hz, J₂=12.0 Hz), 4.92 (2H, s), 6.22 (1H, d, J=4.5 Hz), 6.32 (H, t, J=4.8 Hz), 6.37 (1H, t, J=4.8 Hz), 6.97 (2H, s), 7.38 (6H, g, J=7.8 Hz), 7.55 (3H, dq, J₁=1.1 Hz, J₂=4.8 Hz), 7.55 (3H, q, J=6.2 Hz), 7.81 (1H, s), 7.94 (4H, dt, J₁=1.3 Hz, J₂=6.9 Hz), 8.00 (2H, dd, J₁=1.3 Hz, J=7.2 Hz); MS (M+H⁺): 778.2.

For coupling at the (6)-O-position, the 2′,3′,5′-tri-O-benzoyl-6-O-[(2,4,6-trimethylphenyl)sulfonyl]-guanosine (I-22, 1 g, 1.28 mmol) and 1,4-diazabicyclo[2.2.2]octane (DABCO, 300 mg, 2.56 mmol) were dissolved in CH₂Cl₂ (25 mL) containing activated 3 Å molecular sieves prior to the addition of the selected alkyl alcohol ROH (12.8 mmol). After 30 min of room temperature stirring, 1,8-diazabicyclo[5.4.0]undec-7-ene (DBU, 300 μL, 1.92 mmol) was added to the solution and stirring was applied for another 18 h. Silica gel (20 g) was added to the suspension and the solvents were removed in vacuo. Following column chromatography (EtOAc/hexanes) the intermediate product was immediately dissolved in MeOH (5 mL) containing NaOH 1 M (0.1 mL) and the resulting solution was stirred at room temperature for 90 min to deprotect the ribosyl hydroxyl groups. After removal of the solvents in vacuo, the residue was purified by column chromatography (10% MeOH/CHCl₃) to afford the corresponding (6)-O-modified guanosine compositions I-24 in 39-79% yield. Exemplary compositions prepared by this method include, but are not limited to:

6-O-methyl guanosine (I-24a). 39% yield. Rf: 0.31 (10% MeOH/CHCl₃). ¹H NMR (CD₃OD): δ 3.73 (2H, dd, J₁=2.3 Hz, J₂=12.4 Hz), 3.99 (3H, s), 4.15 (1H, d, J=2.4 Hz), 4.34 (1H, dd, J₁=2.3 Hz, J₂=5.1 Hz), 4.77 (1H, t, J=5.6 Hz), 5.83 (1H, d, J=6.5 Hz), 7.95 (1H, s); ¹³C NMR (CD₃OD): δ 54.5, 63.7, 72.9, 75.0, 88.0, 91.1, 116.1, 140.5, 153.9, 161.4, 162.7; MS (M+H⁺): 298.1.

6-O-(2′-methyl propyl)guanosine (I-24b). 53% yield. Rf: 0.31 (10% MeOW/CHCl₃). ¹H NMR (CD₃OD): δ 0.99 (6H, d, J=6.7 Hz), 2.09 (1H, qu, J=6.5 Hz), 3.73 (1H, d, J=12.1 Hz), 4.14-4.20 (3H, m), 4.34 (1H, s) 4.77 (1H, t, J=5.4 Hz), 5.84 (1H, d, J=6.3 Hz), 7.97 (1H, s); ¹³C NMR (CD₃OD): δ 18.5, 18.6, 28.1, 62.6, 71.8, 72.9, 74.0, 87.0, 90.1, 115.2, 139.5, 153.0, 160.3, 161.7; MS (M+H⁺): 340.2.

6-O-allyl guanosine (I-24c). 79% yield. Rf: 0.36 (10% MeOH/CHCl₃). ¹H NMR (CD₃OD): δ 3.75 (1H, dd, J₁=3.7 Hz, J₂=12.6 Hz), 3.85 (1H, dd, J₁=2.9 Hz, J₂=12.0 Hz), 4.09 (1H, q, J=3.5 Hz), 4.34 (1H, t, J=4.6 Hz), 4.65 (1H, t, J=5.0 Hz), 5.08 (2H, d, J=5.3 Hz), 5.30 (1H, d, J=11.0 Hz), 5.48 (1H, d, J=17.0 Hz), 6.02 (1H, d, J=5.1 Hz), 6.14 (1H, m, J=6.0 Hz), 8.41 (1H, s); MS (M+H⁺): 324.1.

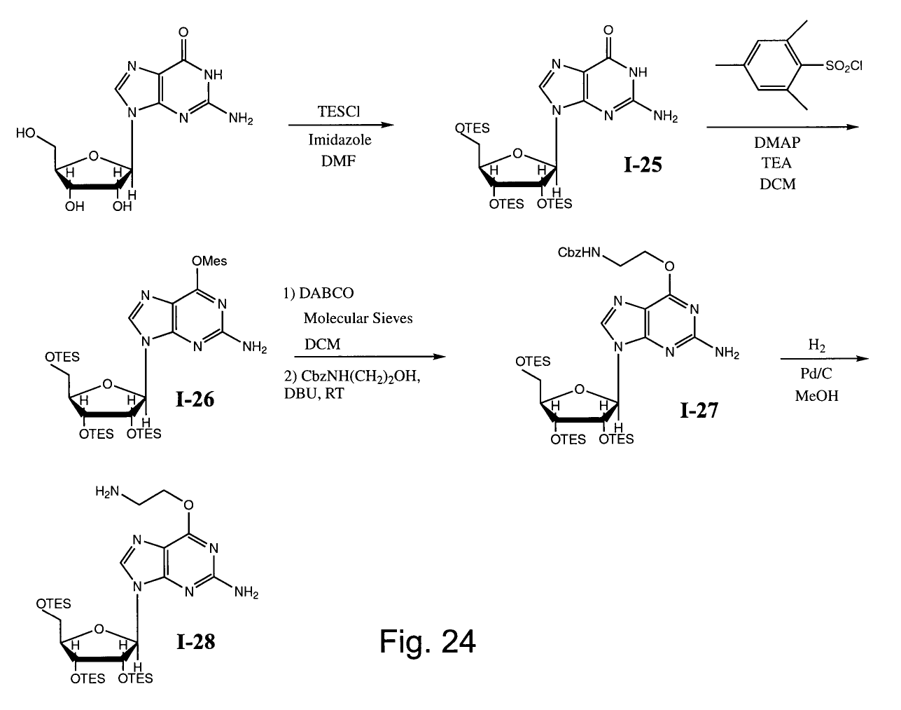

In an alternate embodiment, 2′,3′,5′-tris(O-triethylsilyl)guanosine (I-25) is used as the guanosine substrate for the synthesis of the (6)-O-substituted modulator compounds (FIG. 24). This alternative (6)-O-activated substrate is prepared as follows.

### Exemplary Synthesis: Alternative Preparation Method for (6)-O-modified Guanosines

Guanosine (286 mg, 1 mmol) and imidazole (850 mg, 12 mmol) were suspended in anhydrous DMF (2 mL). The reaction mixture was cooled to 0° C. prior to the addition of chlorotriethylsilane (0.5 mL, 3 mmol). After 30 min of stirring, another addition of chlorotriethylsilane (0.5 mL, 3 mmol) was conducted to drive the reaction to completion. As the reactants solubilized, the reaction was allowed to warm up to room temperature over the next 3 hours. The solvent was removed in vacuo and the residue was purified by column chromatography (MeOH/CHCl₃) to yield 604 mg of a protected intermediate (I-25, 81% yield) as a white solid. Rf: 0.70 (10% MeOH/CHCl₃). ¹H NMR (CDCl₃): δ 0.64-0.68 (18H, m), 0.87-1.01 (27H, m), 3.75 (1H, dd, J₁=2.2 Hz, J₂=11.3 Hz), 3.94 (1H, dd, J₁=3.5 Hz, J₂=11.3 Hz), 4.08 (1H, q, J=3.5 Hz), 4.30 (1H, t, J=4.3 Hz), 4.49 (1H, t, J=4.0 Hz), 5.84 (1H, J=4.2 Hz), 6.32 (2H, brs), 7.90 (1H, s), 11.99 (1H, brs); MS (M+H⁺): 626.3.

The 2′,3′,5′-tris(O-triethylsilyl)guanosine (I-25, 550 mg, 0.74 mmol) and DMAP (9 mg, 0.074 mmol) were suspended in CH₂Cl₂. After the addition of TEA (512 μL, 3.7 mmol), the reaction mixture was chilled to 0° C. for 15 min. The addition of (2,4,6-trimethylphenyl)sulfonyl chloride (357 mg, 1.63 mmol) was followed by stirring at 0° C. for 4 h after which the reaction mixture was allowed to warm up to room temperature over 18 h. Silica gel (5 g) was added to the pink solution and the solvents were removed in vacuo. Column chromatography (EtOAc/hexanes) afforded 368 mg of 2′,3′,5′-Tris(O-triethylsilyl)-6-O-[(2,4,6-trimethylphenyl)sulfonyl]-guanosine (I-26, 62% yield) as a pale yellow solid. Rf: 0.45 (10% EtOAc/hexanes). ¹H NMR (CD₃OD): δ 0.38-0.58 (6H, m), 0.61-0.66 (12H, m) 0.81 (9H, t, J=8.0 Hz), 0.94-0.98 (18H, m), 2.29 (3H, s), 2.73 (6H, s), 3.73 (1H, dd, J₁=2.3 Hz, J₂=11.4 Hz), 3.89 (1H, dd, J₁=3.5 Hz, J₂=11.3 Hz), 4.06 (1H, q, J=2.7 Hz), 4.27 (1H, t, J=3.9 Hz), 4.51 (1H, t, J=4.4 Hz), 4.87 (2H, s), 5.89 (1H, d, J=5.0 Hz), 6.97 (2H, s), 8.07 (1H, s); MS (M+H⁺): 808.5.

Next, the 2′,3′,5′-tris(O-triethylsilyl)-6-O-[(2,4,6-trimethylphenyl)sulfonyl]-guanosine (I-26, 245 mg, 0.30 mmol) and DABCO (67 mg, 0.60 mmol) were dissolved in CH₂Cl₂ (20 mL) containing activated 3 Å molecular sieves prior to the addition of alcohol (585 mg, 3.0 mmol). In the embodiment provided in FIG. 24, benzyl-N-(2-hydroxyethyl)carbamate was employed as the alcohol for coupling to the (6)-position. After 30 min of room temperature stirring, DBU (67 μL, 0.45 mmol) was added to the solution and stirring was applied for another 18 h. Silica gel (5 g) was added to the suspension and the solvents were removed in vacuo. Column chromatography (EtOAc/hexanes) afforded 184 mg of 2′,3′,5′-Tris(O-triethylsilyl)-6-O-[2-N-(benzylcarbamate)ethyl]guanosine (I-27, 88% yield) as a white solid. Rf: 0.50 (50% EtOAc/hexanes).

The 2′,3′,5′-tris(O-triethylsilyl)-6-O-[2-N-(benzylcarbamate)ethyl]guanosine (I-27, 184 mg, 0.23 mmol) was dissolved in EtOAc (10 mL). Following the addition of Pd/C (20 mg), the reaction vessel was repeatedly evacuated and backfilled with H₂. The reaction mixture was then stirred at room temperature for 18 h under an H₂ atmosphere. Filtration of the mixture through a cotton plug, removal of the solvent in vacuo and column chromatography (MeOH/CHCl₃) afforded 158 mg of 2′,3′,5′-Tris(O-triethylsilyl)-6-O-(2″-amino)ethyl guanosine (I-28, 100% yield). Rf: 0.37 (10% MeOH/CHCl₃).

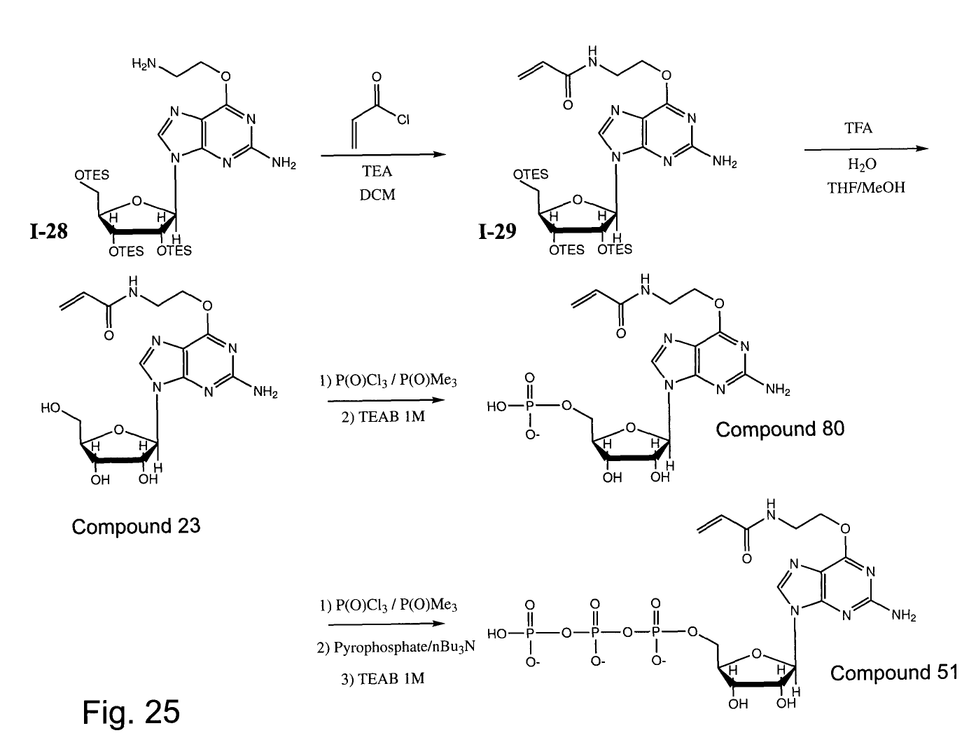

In some cases, the amino group at the 2″ position of the O-ethyl linker was further functionalized to form additional modulator compositions (FIG. 25). For example, the protected compound 2′,3′,5′-tris(O-triethylsilyl)-6-O-(2-amino)ethyl guanosine (I-28, 160 mg, 0.24 mmol) was dissolved in CH₂Cl₂ (5 mL) and TEA (62 μL, 0.45 mmol) was added before cooling the solution to 0° C. Acryloyl chloride (18.7 μL, 0.23 mmol) was added, and the resulting solution was stirred at 0° C. for 1 h and then allowed to warm up to room temperature. To drive the reaction to completion, an optional additional aliquot of acryloyl chloride (10 μL, 0.12 mmol) was added to the reaction mixture. After another 15 min of stirring, the solvent was removed in vacuo and the residue purified by column chromatography (MeOH/CHCl₃) to yield 170 mg of 2′,3′,5′-Tris(O-triethylsilyl)-6-O-(2-N-acrylamido)ethyl guanosine (I-29, 98% yield) as a white solid. Rf: 0.72 (10% MeOH/CHCl₃). ¹H NMR (CDCl₃): δ 0.38-0.58 (6H, m), 0.64-0.72 (12H, m) 0.83 (9H, t, J=8.0 Hz), 0.95-1.00 (18H, m), 3.75 (1H, dd, J₁=2.3 Hz, J₂=11.3 Hz), 3.80 (2H, q, J=4.7 Hz), 3.92 (1H, dd, J₁=3.4 Hz, J₂=11.3 Hz), 4.07 (1H, q, J=2.7 Hz), 4.29 (1H, t, J=3.8 Hz), 4.52 (1H, t, J=4.8 Hz), 4.59 (2H, t, J=4.7 Hz), 5.12 (1H, brs), 5.62 (1H, d, J=10.2 Hz), 6.00 (1H, d, J=5.2 Hz), 6.10 (1H, m), 6.28 (1H, dd, J₁=1.1 Hz, J₂=16.9 Hz), 6.49 (1H, brs), 8.01 (1H, s); MS (M+H⁺): 723.4.

To a solution of 2′,3′,5′-Tris(O-triethylsilyl)-6-O-(2-N-acrylamido)ethyl guanosine (I-29, 148 mg, 0.20 mmol) in THF (4 mL), MeOH (4 mL) and water (0.05 mL) was added TFA (0.2 mL). The resulting solution was stirred at room temperature for 36 h. After removal of the solvents in vacuo, the residue was purified by preparative TLC (10% MeOH/CHCl₃) to afford 66 mg of the deprotected product 6-O-(2″-N-acrylamido)ethyl guanosine (23, 87% yield) as a white solid. Rf: 0.27 (10% MeOH/CHCl₃). ¹H NMR (CD₃OD): δ 3.70 (2H, t, J=5.4 Hz), 3.73 (1H, dd, J₁=2.7 Hz, J₂=12.5 Hz), 3.86 (1H, dd, J₁=2.6 Hz, J₂=12.5 Hz), 4.13 (1H, q, J=2.6 Hz), 4.30 (1H, dd, J₁=2.6 Hz, J₂=5.0 Hz), 4.56 (2H, t, J=5.3 Hz), 4.71 (1H, t, J=5.2 Hz), 5.66 (1H, dd, J₁=3.2 Hz, J₂=8.8 Hz), 5.85 (1H, d, J=6.4 Hz), 6.22-6.30 (2H, m), 8.04 (1H, s).

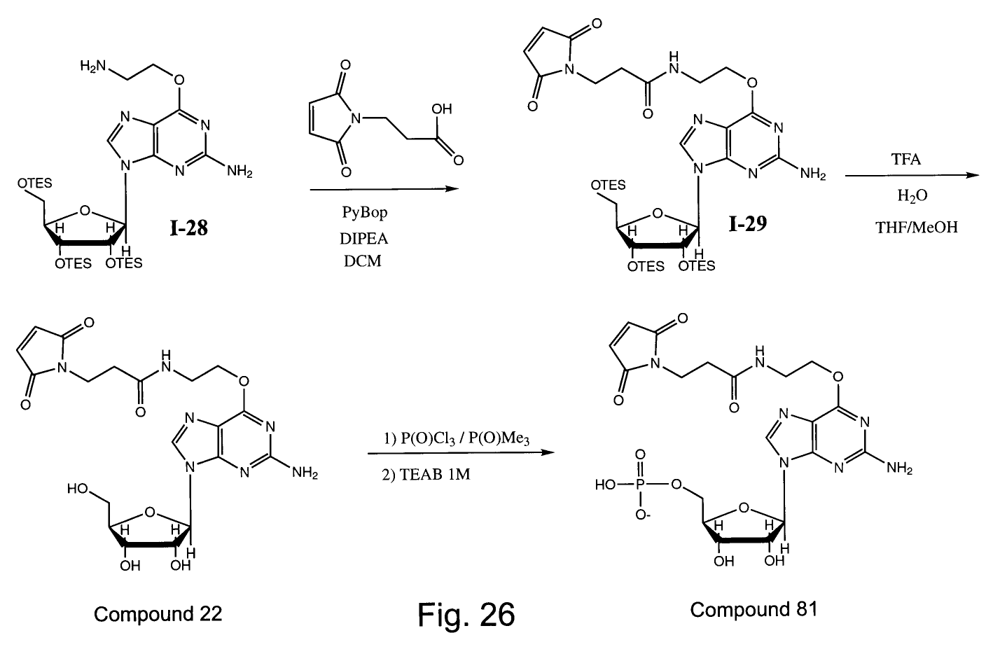

Maleimide-containing GTPase modulators were prepared from the protected compound 2′,3′,5′-tris(O-triethylsilyl)-6-O-(2-amino)ethyl guanosine (I-28) as follows (FIG. 26). The 2′,3′,5′-tris(O-triethylsilyl)-6-O-(2″-amino)ethyl guanosine (25 mg, 0.037 mmol), 3-maleimido propionic acid (11 mg, 0.065 mmol) and PyBop (34 mg, 0.065 mmol) were dissolved in CH₂Cl₂ (2 mL). The resulting solution was cooled down to 0° C. prior to the addition of DIPEA (12.2 μL, 0.07 mmol) and was then stirred at 0° C. for 18 h. The solvent was removed in vacuo and the residue purified by preparative TLC (5% MeOH/CHCl₃) to afford 27.5 mg of 2′,3′,5′-Tris(O-triethylsilyl)-6-O-{[(3″-maleimido)propylamido]ethyl}guanosine (I-29, 90% yield) as a bright yellow solid. Rf: 0.75 (10% MeOH/CHCl₃). ¹H NMR (CDCl₃): δ 0.40-0.50 (6H, m), 0.62-0.68 (12H, m) 0.85 (9H, t, J=8.0 Hz), 0.95-1.00 (18H, m), 2.65 (2H, t, J=6.8 Hz), 3.74 (1H, dd, J₁=2.9 Hz, J₂=11.2 Hz), 3.85 (4H, t, J=6.8 Hz), 3.93 (1H, dd, J₁=4.2 Hz, J₂=11.2 Hz), 4.05 (1H, q, J=3.2 Hz), 4.27 (2H, t, J=5.3 Hz), 4.31 (1H, t, J=4.0 Hz), 4.66 (3H, brs), 5.85 (1H, d, J=5.0 Hz), 5.94 (1H, brs), 6.69 (2H, s), 7.81 (1H, s); MS (M+H⁺): 820.5.

The 2′,3′,5′-tris(O-triethylsilyl)-6-O-{[(3″-maleimido)propylamido]ethyl}guanosine (I-29, 25 mg, 0.030 mmol) was then dissolved in MeOH (5 mL) and TFA (0.4 mL) was added. The resulting solution was stirred at room temperature for 20 h. Following the removal of the solvents in vacuo, the residue was purified by preparative TLC (7% MeOH/CHCl₃) to afford 12.7 mg of the deprotected compound 6-O-{[(3″-maleimido)propylamido]ethyl}guanosine (22, 87% yield) as a bright yellow solid. Rf: 0.31 (10% MeOH/CHCl₃). ¹H NMR (CD₃OD): δ 2.61 (2H, t, J=6.9 Hz), 3.72 (1H, dd, J₁=2.4 Hz, J₂=12.6 Hz), 3.74 (4H, t, J=6.7 Hz), 3.87 (1H, dd, J₁=2.3 Hz, J₂=12.5 Hz), 4.14 (1H, q, J=2.2 Hz), 4.26 (2H, t, J=5.4 Hz), 4.28 (1H, dd, J₁=1.9 Hz, J₂=5.0 Hz), 4.74 (1H, dt, J₁=1.6 Hz, J₂=6.0 Hz), 5.78 (1H, d, J=6.8 Hz), 6.74 (2H, d, J=4.1 Hz), 7.85 (1H, s); MS (M+H⁺): 478.1.

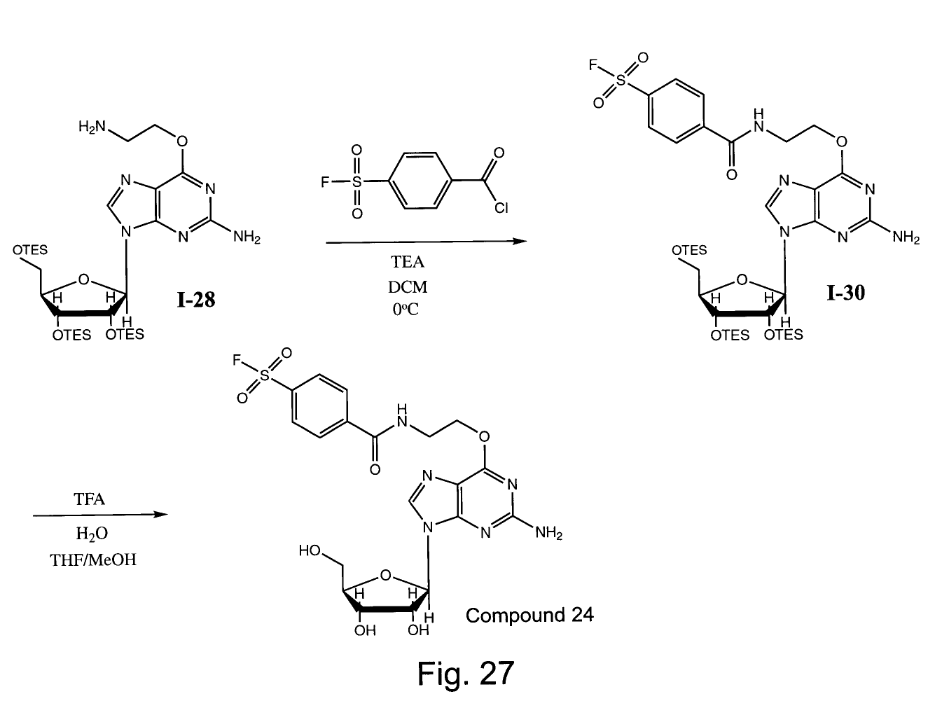

In yet another embodiment (FIG. 27), 2′,3′,5′-Tris(O-triethylsilyl)-6-O-(2-amino)ethyl guanosine (I-28, 935 mg, 1.4 mmol) and TEA (276 μL, 2.0 mmol) were dissolved in CH₂Cl₂ (20 mL). The resulting solution was cooled down to 0° C. prior to the addition of p(fluorosulfonyl) benzoyl chloride in a CH₂Cl₂ (4 mL) solution and was then stirred at 0° C. for an additional 2 h. The solvent was removed in vacuo and the residue purified by column chromatography (EtOAc/hexanes) to afford 1.065 g of the protected intermediate 2′,3′,5′-Tris(O-triethylsilyl)-6-O-{[(4′-fluorosulfonyl)benzamido]ethyl}guanosine (I-30, 90% yield) as a slightly yellow solid. Rf: 0.81 (10% MeOH/CHCl₃). ¹H NMR (CDCl₃): δ 0.35-0.51 (6H, m), 0.59-0.68 (12H, m) 0.80 (9H, t, J=8.0 Hz), 0.95 (18H, dt, J₁=1.2 Hz, J₂=13.1 Hz), 3.73 (1H, dd, J₁=2.7 Hz, J₂=11.2 Hz), 3.90 (1H, dd, J₁=4.0 Hz, J₂=11.2 Hz), 3.93 (2H, brs), 4.06 (1H, q, J=3.1 Hz), 4.27 (1H, t, J=3.7 Hz), 4.51 (2H, t, J=5.2 Hz), 4.56 (1H, t, J=4.8 Hz), 5.06 (2H, brs), 5.87 (1H, d, J=5.4 Hz), 6.58 (1H, t, J=5.8 Hz), 7.83 (1H, s), 8.00 (2H, d, J=8.5 Hz), 8.16 (2H, d, J=8.3 Hz); MS (M+H⁺): 855.3.

To a solution of 2′,3′,5′-Tris(O-triethylsilyl)-6-O-{[(4-fluorosulfonyl)benzamido]ethyl}guanosine (I-30, 15 mg, 0.017 mmol) in THF (1 mL), MeOH (1 mL) and water (0.05 mL) was added TFA (0.5 mL). The resulting solution was stirred at room temperature for 3 h. After removal of the solvents in vacuo, the residue was purified by preparative TLC (15% MeOH/CHCl₃) to afford 3.5 mg of 6-O-{[(4′-fluorosulfonyl)benzamido]ethyl}guanosine (24, 39% yield) as a yellowish glassy solid. Rf: 0.36 (10% MeOH/CHCl₃). MS (M+H⁺): 513.1

### General synthetic method for (6)-O-alkylmercapto guanosines

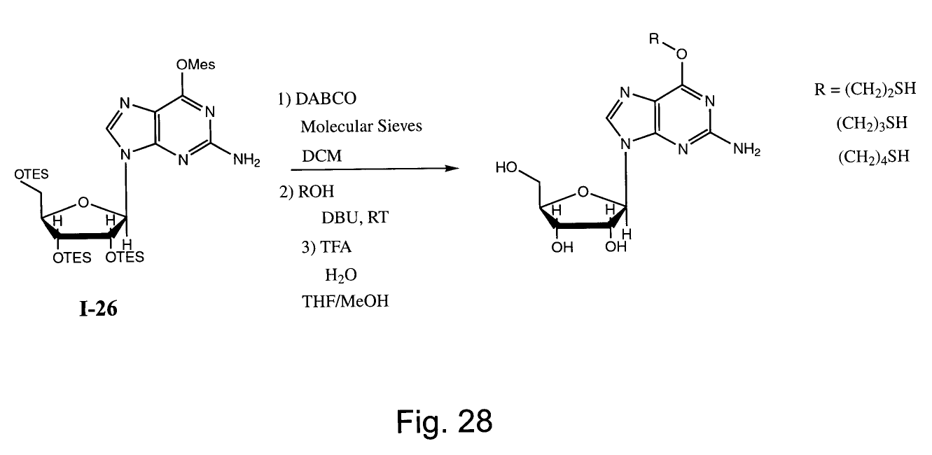

As another example, the present invention provides methods for synthesizing alkylmercapto-containing GTPase modulators (FIG. 28).

### Exemplary Synthesis: Preparation of (6)-O-alkylmercapto guanosines

2′,3′,5′-Tris(O-triethylsilyl)-6-O-[(2,4,6-trimethylphenyl)sulfonyl]-guanosine (I-26, 1 g, 1.23 mmol) and DABCO (276 mg, 2.43 mmol) were dissolved in CH₂Cl₂ (40 mL) containing activated 3 Å molecular sieves prior to the addition of the mercaptoalkyl alcohol (12.3 mmol). After 30 min of room temperature stirring, DBU (275 μL, 1.845 mmol) was added to the solution and stirring was applied for another 18 h. Silica gel (5 g) was added to the suspension and the solvents were removed in vacuo. Rapid column chromatography (EtOAc/hexanes) afforded the protected products as in intermediates. To a solution of these intermediates in THF (10 mL) and MeOH (10 mL) was added TFA (1 mL), the resulting solution was stirred at room temperature for 2 h. After removal of the solvents in vacuo, the residue was purified by column chromatography (10% MeOH/CHCl₃) to afford compounds 27-29 (39-76% yield). Exemplary GTPase modulator compounds prepared by this method include:

(6)-O-(2′-mercapto)ethyl guanosine (27). (39% yield). Rf=0.25 (10% MeOH/CHCl₃). ¹H NMR (CD₃OD): δ 3.42 (2H, t, J=6.5 Hz), 3.73 (1H, dd, J₁=2.9 Hz, J₂=12.4 Hz), 3.80 (2H, t, J=6.6 Hz), 3.86 (1H, dd, J₁=2.7 Hz, J₂=12.4 Hz), 4.11 (1H, q, J=2.8 Hz), 4.30 (1H, dd, J₁=2.8 Hz, J₂=5.1 Hz), 4.68 (1H, dt, J₁=0.8 Hz, J₂=5.7 Hz), 5.85 (1H, d, J=6.2 Hz), 8.08 (1H, s); MS (M+H⁺): 344.0.

(6)-O-(3′-mercapto)propyl guanosine (28). (69% yield). Rf=0.26 (10% MeOH/CHCl₃). ¹H NMR (CD₃OD): δ 1.88 (2H, q, J=7.0 Hz), 3.26 (2H, t, J=4.7 Hz), 3.62 (2H, t, J=6.1 Hz), 3.70 (1H, dd, J₁=2.7 Hz, J₂=12.4 Hz), 3.82 (1H, dd, J₁=2.4 Hz, J₂=12.5 Hz), 4.14 (1H, q, J=2.5 Hz), 4.28 (1H, dd, J₁=2.4 Hz, J₂=5.2 Hz), 4.80 (1H, t, J=5.9 Hz), 5.87 (1H, d, J=6.3 Hz), 7.99 (1H, s); MS (M+H⁺): 358.0.

(6)-O-(4′-mercapto)butyl guanosine (29). (76% yield). Rf=0.26 (10% MeOH/CHCl₃). ¹H NMR (CD₃OD): δ 1.61-1.66 (2H, m), 1.71-1.76 (2H, m), 3.18-3.26 (2H, m), 3.56 (2H, t, J=6.3 Hz), 3.74 (1H, dd, J₁=2.5 Hz, J₂=12.3 Hz), 3.87 (1H, dd, J₁=2.5 Hz, J₂=12.5 Hz), 4.17 (1H, brs), 4.39 (1H, brs), 4.83 (1H, t, J=5.5 Hz), 5.90 (1H, d, J=6.3 Hz), 8.03 (1H, s); MS (M+H⁺): 372.0.

### General synthetic method for 6-O-(maleimido)alkyl guanosines

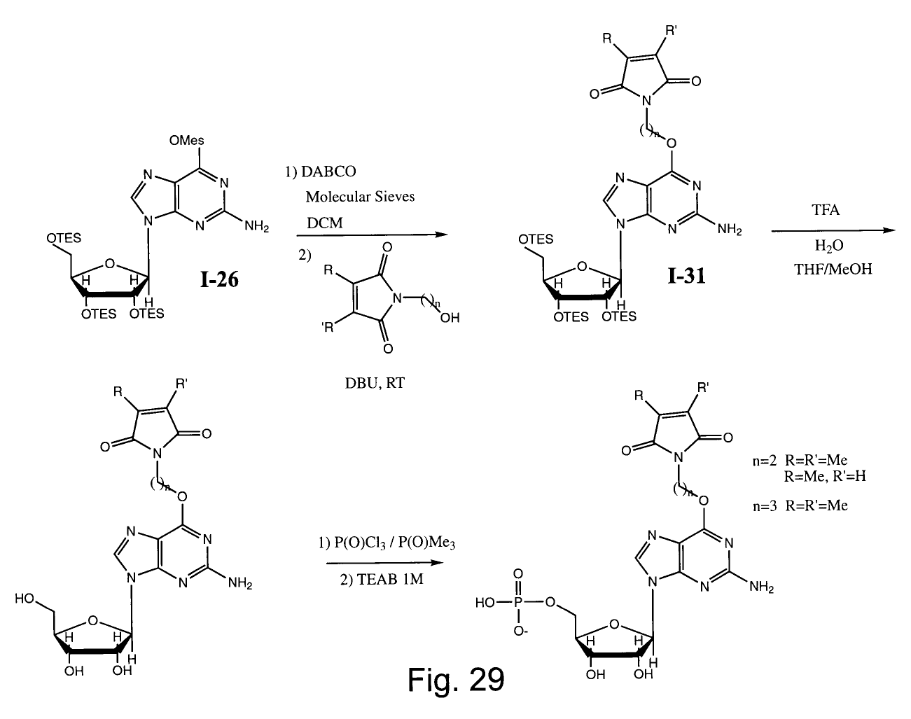

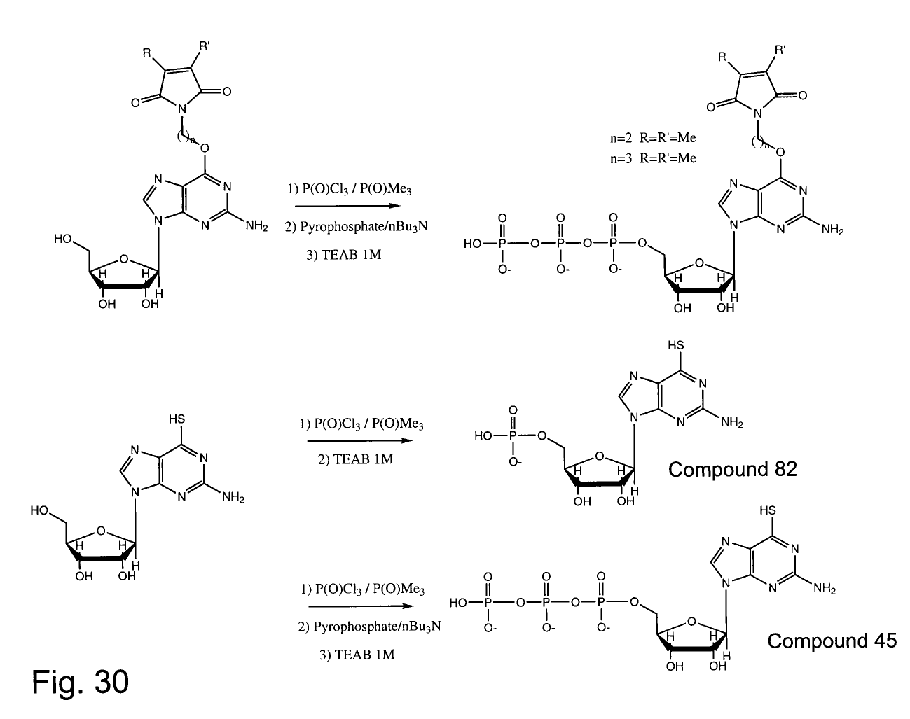

As a further example, the present invention provides methods for synthesizing alkylmaleimido-containing GTPase modulators (FIG. 29).

### Exemplary Synthesis: Preparation of 6-O-(maleimido)alkyl guanosines

2′,3′,5′-Tris(O-triethylsilyl)-6-O-[(2,4,6-trimethylphenyl)sulfonyl]-guanosine (I-26, 900 mg, 1.11 mmol) and DABCO (250 mg, 2.22 mmol) were dissolved in CH₂Cl₂ (20 mL) containing activated 3 Å molecular sieves prior to the addition of the selected maleimide-derived alcohol (11.1 mmol). After 30 min of room temperature stirring, DBU (249 μL, 1.67 mmol) was added to the solution and stirring was applied for another 18 h. Silica gel (5 g) was added to the suspension and the solvents were removed in vacuo. Rapid column chromatography (EtOAc/hexanes) afforded the protected intermediates. To a solution of these intermediates in THF (4 mL), MeOH (14 mL) and water (0.1 mL) was added TFA (0.1 mL), the resulting solution was stirred at room temperature for 18 h. After removal of the solvents in vacuo, the residue was purified by column chromatography (10% MeOH/CHCl₃) to afford the maleimido-containing modulator compounds 36-38 (3.5-68% yield). Exemplary GTPase modulator compounds prepared by this method include:

6-O-[(3″,4″-dimethyl)maleimido]ethyl guanosine (37). (67% yield). Rf=0.49 (10% MeOH/CHCl₃).

6-O-[(3″-methyl)maleimido]ethyl guanosine (36). (3.5% yield). Rf=0.24 (10% MeOH/CHCl₃). ¹H NMR (CD₃OD): δ 2.00 (3H, t, J=1.7 Hz), 3.73 (1H, dd, J₁=2.8 Hz, J₂=12.4Hz), 3.86 (1H, dd, J₁=2.6 Hz, J₂=12.4 Hz), 3.91 (2H, t, J=5.4 Hz), 4.12 (1H, q, J=2.7 Hz), 4.30 (1H, dd, J₁=2.7 Hz, J₂=5.2 Hz), 4.67-4.71 (3H, m), 5.83 (1H, d, J=6.4 Hz), 7.99 (1H, s).

6-O-[(3″,4″-dimethyl)maleimido]propyl guanosine (38). (68% yield). Rf=0.30 (10% MeOH/CHCl₃).

### Synthesis of β-D-ribofuranosyl-pyrazolo[3,4-d]pyrimidine based modulators

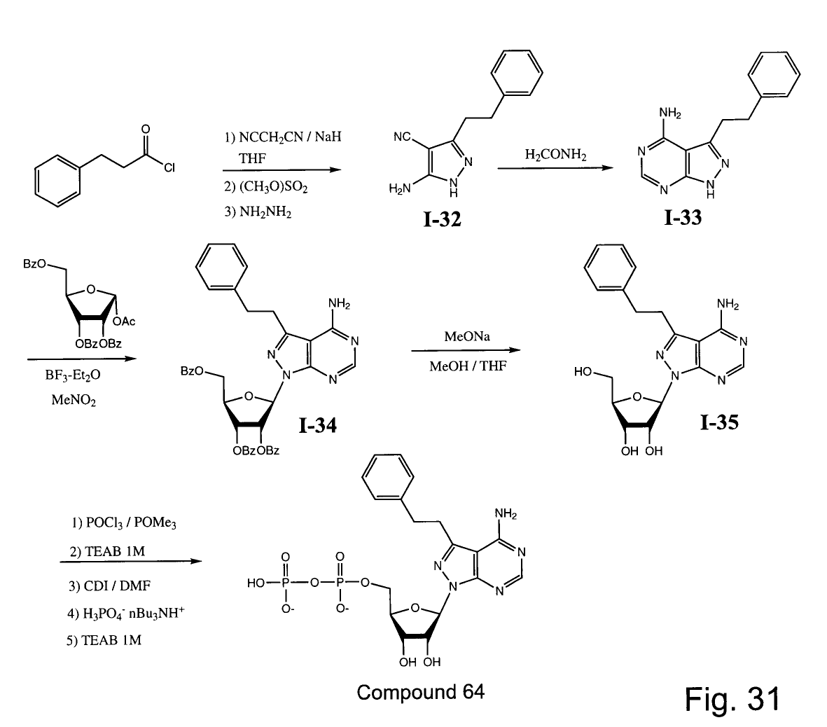

The present invention also provides methods for the preparation of β-D-ribofuranosyl-pyrazolo[3,4-d]pyrimidine based modulators (FIG. 31). The pyrazolo-pyrimidine scaffold structure (similar to the guanosine scaffold except for the reversal in position of the N-7 and C-8 atoms of the pentene ring) can be prepared as follows.

### Exemplary Synthesis: Preparation of β-D-ribofuranosyl-pyrazolo[3,4-d]pyrimidines

Malononitrile (30 mmol) was added slowly to a suspension of sodium hydride (60 mmol) in THF (50 mL). Following the addition of 3-phenylpropyonyl chloride(4.5 mL, 30 mmol), the resulting exothermic reaction was complete after stirring for 1 h. Dimethyl sulfate (40 mmol) was then added and the reaction mixture was stirred at reflux for 5 h and at 25° C. for a further 48 h. After the additions of triethylamine (100 mmol) and hydrazine (30 mmol), the reaction mixture was refluxed for another 5 h. After removal of the solvents in vacuo, extraction of the residue was carried out using aqNaHCO₃ (20 mL), Brine (20 mL) and EtOAc (3×20 mL). The desired compound was purified by column chromatography (EtOAc/hexanes) and recrystallized from MeOH/water to yield intermediate I-32. After drying, I-32 (1 g, 4.7 mmol) was dissolved in formamide (30 mL) and the reaction mixture was refluxed at 180° C. overnight. After cooling to RT, water (60 mL) was added and the resulting precipitate was filtered off. Trituration of the precipitate with EtOAc (100 mL) was followed by a second filtration. Subsequent washes of the filtrate with brine (3×50 mL) and drying of the organic phase with Mg₂SO₄ followed by removal of the solvents in vacuo afforded 980 mg of 4-amino-3-(2′-phenyl)ethylpyrazolo[3,4-d]pyrimidine (intermediate I-33, 87% yield).

Intermediate I-33 (980 mg, 4.1 mmol) was then added to a solution of 1-acetate-2,3,5-tribenzoate ribofuranose (3.1 g, 6.1 mmol) in anhydrous nitromethane (28 mL). The reaction mixture was heated to reflux before adding BF₃-Et₂O (0.76 mL, 6.1 mmol) and then refluxed for 90 min. The solvents were removed in vacuo and the dark oily residue was dissolved in EtOAc (100 mL). After extraction with aqNaHCO₃ (3×100 mL), the organic layer was dried with Mg₂SO₄. The solvent was removed in vacuo and the residue purified by column chromatography (EtOAc/hexanes) to afford 2.1 g of intermediate I-34 (75% yield) as a dark colored foam. Intermediate I-34 (2.1 g, 3.1 mmol) was dissolved in MeOH (25 mL) and THF (5 mL) before adding sodium methoxide (0.5 mL, 25% w/v). The reaction solution was stirred at RT overnight. Filtration of the precipitate afforded 800 mg of nonphosphorylated modulator compound I-35 (68% yield) as a white solid. ¹H NMR (DMSO): δ 3.02 (2H, t, J=8.4 Hz), 3.29 (2H, t, J=8.4 Hz), 3.43 (1H, qu, J=6.4 Hz), 3.60 (1H, qu, J=4.4 Hz), 3.92 (1H, q, J=5.2 Hz), 4.55 (1H, q, J=5.2 Hz), 4.87 (1H, t, J=5.2 Hz), 5.11 (1H, d, J=5.6 Hz), 5.35 (1H, d, J=6.0 Hz), 6.07 (1H, d, J=4.4 Hz), 7.16-7.20 (1H, m), 7.26-7.31 (5H, m), 7.37 (1H, brs), 8.17 (1H, s); ¹³C NMR (DMSO): δ 31.3, 35.3, 64.4, 72.8, 75.1, 86.9, 90.2, 127.8, 130.0, 130.1, 130.4, 143.0, 147.1, 156.9, 157.7, 160.1; MS (M+H⁺): 372.1. The guanosine modulator compound I-35 can then be phosphorylated to form compound 64 via standard procedures, as discussed below.

### Guanosine mono-, di- and triphosphate-Based Modulators

The GTPase modulators of the present invention also include mono-, di- and tri-phosphate derivatives of the modified guanosine modulators.

### Exemplary Synthesis: Preparation of Guanosine mono-, di- and Triphosphates

The mono-, di- and triphosphate derivatives synthesized were purified, analyzed and characterized, e.g., using a combination of anion-exchange HPLC and negative mode LC-MS. In one embodiment, the HPLC system included a Star Chromatography™ workstation (Varian) coupled to a ProStar™ HPLC unit (Varian) with a Poros anion exchange column (PerSeptive Biosystems, #1-2312-46 HQ110-012X, 10 mm diameter×100 mL length). Samples were applied to the column through a 2 mL injection loop and were submitted to the following elution conditions: gradient of 0 to 100% solvent A (1 M TEAB) over the first 8 min and isocratic elution with 100% solvent A from 8 to 14 min. The system was then re-equilibrated with 100% solvent B (water). Under these conditions, the retention times of phosphate-bearing species were typically about 5 min for monophosphates, about 6.5 min for diphosphates and about 8 min for triphosphates.

### General synthetic method for (6)- and C(7)-modified guanosine (5′)-monophosphate derivatives

Any of the guanosine compositions provided herein are contemplated for use in the preparation of modulators having a phosphorylated guanosine scaffold. In one embodiment, the GMP-based modulator compositions of the present invention are prepared from the corresponding guanosine modulator compound as follows.

### Exemplary Synthesis: Preparation of (6)- and C(7)-modified GMP derivatives

The selected guanosine derivative (0.20 mmol) was dissolved, or suspended, in trimethyl phosphate (0.5 mL) and the reaction mixture was cooled down to 4° C. in an ice bath. Phosphorus oxychloride (0.25 mmol) was added and the reaction is allowed to proceed for 2 h at 4° C. under a nitrogen atmosphere. Quenching of the reaction was provided by the addition of 5 mL of ice cold 1 M triethylammonium bicarbonate (TEAB) pH 7.5. The solvents were removed rapidly in vacuo and the white crystalline material was resuspended in 0.025 M TEAB (2 mL). The crude product was then applied to a DEAE cellulose column (Aldrich, #2145), rinsed with 10 column volumes of 0.025 M TEAB and eluted in 2 column volumes of 1 M TEAB. The solvent was removed rapidly in vacuo and the purity of the compound was evaluated by HPLC at 254 nm and by LC-MS. Further purification of the compound was achieved by HPLC as described above. For testing, an aqueous solution of a guanosine 5′-monophosphate derivative was prepared and its concentration determined by comparing its absorption at 280 nm to those of standard solutions of the corresponding guanosine derivative.

Exemplary GTPase modulators based upon the GMP-scaffold include, but are not limited to, the following compositions:

6-S-mercaptoguanosine-5′-phosphate (82). (3.5% yield). Rt=5.234 min; MS (M−H): 499.1.

6-O-{[(3-maleimido)propylamido}ethyl guanosine-5′-phosphate (81). (3% yield). Rt=4.781 min; MS (M−H): 556.2.

6-O-(2-N-acrylamido)ethyl guanosine-5′-phosphate (80). (13% yield). Rt=5.384 min; MS (M−H): 459.1

6-O-[(3″-methyl)maleimido]ethyl guanosine-5′-phosphate (83). (7% yield). Rt=5.234 min; MS (M−H): 499.1.

6-O-[(3″,4″-dimethyl)maleimido]ethyl guanosine-5′-phosphate (84). (55% yield), Rt=4.745 min; MS (M−H): 513.1.

6-O-[(3″,4″-dimethyl)maleimido]propyl guanosine-5′-phosphate (85). (84% yield). Rt=5.194 min; MS (M−H): 527.1.

### General synthetic method for (6)- and C(7)-modified guanosine (5′)-diphosphate derivatives

In another embodiment, GDP-based modulator compositions are prepared from the corresponding GMP modulator compound as follows.

### Exemplary Synthesis: Preparation of (6)- and C(7)-modified GDP derivatives

A guanosine 5′-monophosphate derivative (0.15 mmol) was dissolved in dry ethanol and the solvent was removed in vacuo to remove water; this step was repeated three times. The solid was further dried by overnight storage in vacuo above P₂O₅. The monophosphate compound was dissolved, or suspended, in 1 mL DMF. A solution of carbonyl diimidazole (0.75 mmol) in 1 mL DMF was added and the reaction mixture was stirred for 24 h at 25° C. under a nitrogen atmosphere. Methanol (0.60 mmol) was added to the mixture and allowed to quench the remaining carbonyl diimidazole for 1 h at 25° C. Phosphoric acid (1.5 mmol) and tri-n-butylamine (1.65 mmol) were dissolved in water and the solvent was removed in vacuo. The salt obtained was then dried in the same fashion as the guanosine 5′-monophosphate derivative. A 1 mL DMF solution of this salt was added to the reaction mixture and stirring was applied for another 24 h at 25° C. Ice cold 1 M TEAB (5 mL) was used to stop the reaction. After stripping the solvents in vacuo, the crude product was resuspended in 2 mL of 0.025 M TEAB and applied to a DEAE column. The product was first washed with 10 column volumes of 0.025 M TEAB and eluted using a gradient of 0.025 M TEAB to 1 M TEAB over 50 column volumes. The solvent was removed rapidly in vacuo and the purity of the compound was evaluated by HPLC at 254 nm and by LC-MS. Optionally, further purification of the compound was achieved by HPLC as described herein.

For testing, an aqueous solution of a guanosine 5′-diphosphate derivative was prepared and its concentration determined by comparing its absorption at 280 nm to those of standard solutions of the corresponding guanosine derivative. Exemplary GTPase modulators having structures based upon the GDP-scaffold include, but are not limited to, the following compositions:

4-amino-3-(2″-phenyl)ethyl-1-β-D-ribofuranosylpyrazolo[3,4-d]pyrimidine-5′-diphosphate (64). (17% yield). Rt=7.268 min; MS (M−H): 530.1.

7-deaza-7-(3″,3″-dimethyl)butyl guanosine-5′-diphosphate (58). (33% yield). Rt=7.852 min; MS (M−H): 525.0.

7-deaza-7-(3″-methyl)butyl guanosine-5′-diphosphate (57). (26% yield).

7-deaza-7-(2″-cyclohexyl)ethyl guanosine-5′-diphosphate (59). (7% yield). Rt=8.300 min; MS (M−H): 551.1.

7-deaza-7-(2″-phenyl)ethyl guanosine-5′-diphosphate (60). (16% yield). Rt=9.960 min; MS (M−H): 545.2.

7-deaza-7-[2″-(4-fluoro)phenyl]ethyl guanosine-5′-diphosphate (61). (22% yield). Rt=11.049 min; MS (M−H): 563.2

7-deaza-7-(2″-phenyl)propyl guanosine-5′-diphosphate (62). (12% yield). Rt=10.799 min; MS (M−H): 559.1

7-deaza-7-(2″-(1-naphtyl)ethyl guanosine-5′-diphosphate (63). (72% yield). Rt=11-22 min; MS (M−H): 595.1.

### General synthetic method for (6)- and C(7)-modified guanosine (5′)-triphosphate derivatives

In a further embodiment, GTP-based modulator compositions are prepared from the corresponding guanosine modulator compound as follows.

### Exemplary Synthesis: Preparation of (6)- and C(7)-modified GTP derivatives

The selected guanosine derivative (0.20 mmol) was dissolved, or suspended, in trimethyl phosphate (0.5 mL) and the reaction mixture was cooled down to 4° C. in an ice bath. Phosphorus oxychloride (0.25 mmol) was added and the reaction is allowed to proceed for 2 h at 4° C. under a nitrogen atmosphere. Pyrophosphate (1.0 mmol) and tri-n-butylamine (2.1 mmol) were dissolved in water and the solvent was removed in vacuo. The salt obtained was then dried through repeated additions and removal of dry EtOH in vacuo, followed by overnight storage in vacuo above P₂O₅. A 1 mL DMF solution of this salt was added quickly to the reaction mixture and stirring was applied for 1 min. The resulting solution was immediately poured into 10 mL of ice cold 1 M TEAB and the solvents were removed in vacuo. The crude product was resuspended in 2 mL of 0.025 M TEAB and applied to a DEAE column. The product was first washed with 10 column volumes of 0.025 M TEAB and eluted using a gradient of 0.025 M TEAB to 1 M TEAB over 50 column volumes. The solvent was removed rapidly in vacuo and the purity of the compound was evaluated by HPLC at 254 nm and by LC-MS. Optionally, further purification of the compound was achieved by HPLC as described herein.

For testing, an aqueous solution of a guanosine 5′-triphosphate derivative was prepared and its concentration determined by comparing its absorption at 280 nm to those of standard solutions of the corresponding guanosine derivative. Exemplary GTPase modulators based upon the GTP-scaffold include, but are not limited to, the following compositions:

6-O-methyl guanosine-5′-triphosphate (44). (2% yield). Rt=8.100 min; MS (M−H): 536.0

6-O-allyl guanosine-5′-triphosphate (43). (1% yield). Rt=8.654 min.

6-O-(2″-methyl)propyl guanosine-5′-triphosphate (40).

6-N-benzyl guanosine-5′-triphosphate (39).

6-N-(2″-methyl)propyl guanosine-5′-triphosphate (42).

6-N-(2″-phenyl)ethyl guanosine-5′-triphosphate (41).

6-S-mercaptoguanosine-5′-triphosphate (45). (1% yield). Rt=7.976 min (free thiol) and 10.449 min (disulfide); MS (M−H): 538.0.

6-O-[(3″,4″-dimethyl)maleimido]ethyl guanosine-5′-triphosphate (49). (20% yield), Rt=7.480 min.

6-O-[(3″,4″-dimethyl)maleimido]propyl guanosine-5′-triphosphate (50). (37% yield). Rt=8.066 min; MS (M−H): 687.0.

6-O-(2-N-acrylamido)ethyl guanosine-5′-triphosphate (51). (1% yield). Rt=8.226 min; MS (M−H): 619.0

### Screening Assays

A wide variety of protein function assays, phenotype-based cellular assays, and/or cellular signaling assays can be employed to examine the GTPase modulators, including, but not limited to, assays which detect the release of a phosphate group, assays which detect thermodynamic changes due to binding interactions, assays that monitor a downstream signaling event via an effector molecule, and the like.

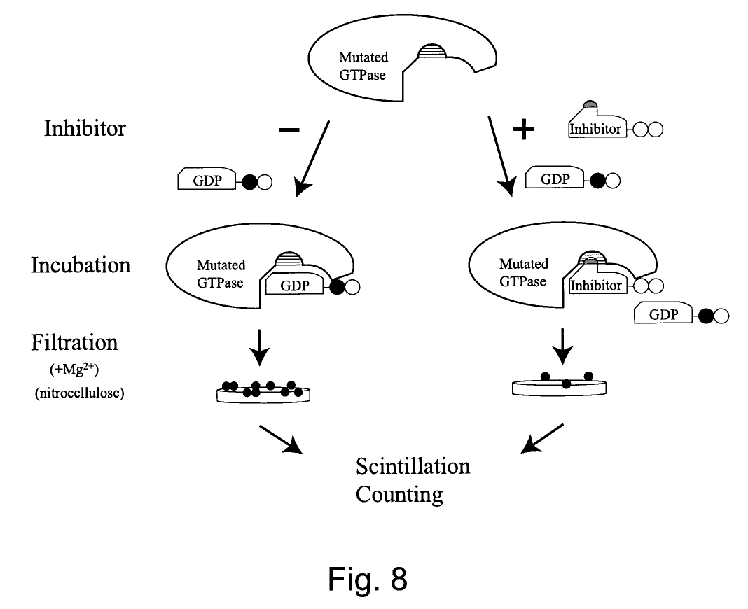

One exemplary assay which can be used are Ras “inhibition” or “guanine nucleotide exchange” assays, as schematically illustrated in FIG. 8, and described in detail in the Examples hereinbelow. The assay detects inhibition of wild-type and mutant Ras proteins by using ³H-labelled GDP and cold (non-tritiated) synthetic Ras inhibitors.

### Methods of Using Modified GTPases and GTPase Modulations

The modified GTPases, GTPase modulators, and mutant GTPase/modulator complexes of the present invention can be used in a number of manners. For example, the mutant GTPases can be used to identify cellular components that interact with or are regulated by the GTPase “switch” mechanism; to examine the biochemical and/or phenotypic response of the cell to changes in GTPase activity; to provide a mechanism for quantitative measurement of the activity of a specific GTPase; to ascertain how known (or putative) pharmaceutical products affect signal transduction, protein synthesis, or other GTPase-mediated cellular activities; and as therapeutic approaches (such as gene therapy) to treat GTPase-mediated disease conditions. Additional uses for the compositions and methods of the present invention will readily become apparent to one of skill in the art.

Typically, the methods of the present invention include the step of providing a mutant GTPase, which differs from a wild-type GTPase by one or more non-native amino acids. The wild-type GTPase can be a GTPase naturally found in the cell, or it can be an (unmodified) sequence engineered into the cell for analysis. While the mutant GTPase(s) employed in the method generally retains its ligand specificity (e.g., no change in functionality), the mutant optionally can have a decreased affinity for GTP and/or GDP, as compared to the parent enzyme. Any of the mutant GTPases of the present invention are contemplated for use in these (and other) methods described herein.

Expression of the mutant GTPase can be achieved by any of a number of methodologies known to one of skill in the art. For example, a nucleic acid construct encoding the mutant GTPase can be cloned into a plasmid or expression cassette, and transfected into the host cell by standard methodologies (e.g., electroporation, microinjection, particle bombardment, polyethylene glycol-mediated transformation, and the like). Such methodologies are known in the art (see, for example, Ausubel, Berger and Kimmel, and Sambrook, all supra, as well as Freshney (1994) Culture of Animal Cells, a Manual of Basic Technique, 3rd Edition (Wiley-Liss, New York) and the references cited therein). Optionally, the plasmid or expression vector also includes a selectable marker such as a gene coding for ampicillin, tetracycline, chloramphenicol or kanamycin resistance.

The step of expressing the mutant GTPase can further include providing a nucleic acid sequence encoding the mutant GTPase. In one embodiment, the mutant GTPase embodies an alteration of at least one amino acid sidechain as compared to the wild-type GTPase, and creation of at least one pocket in or near the nucleotide binding site of the mutant GTPase. Optionally, mutating a wild-type GTPase comprises creating a double mutation in the GTPase sequence, which double mutation provides a modulator binding site sufficiently large to allow a bulky substituent of a GTPase modulator to fit the binding site.

The GTPase construct is optionally expressed in either a prokaryotic system or a eukaryotic system (for example, a bacterium, a tissue culture, or an animal cell). Any of a number of host cells can be used to express the mutant GTPase construct, including, but not limited to, various cell lines available from cell repositories such as the American Type Culture Collection (www.atcc.org), the World Data Center on Microorganisms (wdcm.nig.ac.jp), European Collection of Animal Cell Culture (www.ecacc.org) and the Japanese Cancer Research Resources Bank (cellbank.nihs.go.jp), and companies such as Clonetics Corporation (www.clonetics.com). Cells for use in the methods of the present invention include, but are not limited to, mammalian cells (for example, murine, rodent, guinea pig, rabbit, canine, feline, primate or human cells). Preferred cell lines for use in these and other methods of the present invention include fibroblasts, myeloid leukemia cells, Raji cells, human pancreatic cell lines (such as MIAPaCa-2, and PANC-1), human glioma/glioblastoma cell lines (e.g., U-87, U343 and U373 cell lines), epithelial cells, such as mammary epithelial cells (e.g., murine EpH4 cells), endothelial cells, and the like.

Generally, one of skill in the art is fully able to culture and transfect cells from animals, plants, fungi, bacteria and other cells using available techniques. A variety of cell culture media are described in The Handbook of Microbiological Media, Atlas and Parks (eds.) (1993, CRC Press, Boca Raton, Fla.). References describing the techniques involved in bacterial and animal cell culture include Freshney, Culture of Animal Cells, a Manual of Basic Technique, third edition (1994, Wiley-Liss, New York) and the references cited therein; Humason, Animal Tissue Techniques, fourth edition (1979, W.H. Freeman and Company, New York); and Ricciardelli, et al., In Vitro Cell Dev. Biol. (1989) vol. 25, pp.1016-1024. Information regarding plant cell culture can be found in Plant Cell and Tissue Culture in Liquid Systems, by Payne et al. (1992, John Wiley & Sons, Inc. New York, N.Y.);Plant Cell, Tissue and Organ Culture: Fundamental Methods by Gamborg and Phillips, eds. (1995, Springer Lab Manual, Springer-Verlag, Berlin), and is also available in commercial literature such as the Life Science Research Cell Culture Catalogue (1998) from Sigma-Aldrich, Inc. (St Louis, Mo.) and the Plant Culture Catalogue and supplement (1997) also from Sigma-Aldrich.

Optionally, expressing the mutant GTPase further includes reducing the activity or expression of a corresponding wild-type GTPase. In some embodiments, decreased expression of the wild-type GTPase molecule can be accomplished by disrupting expression of the wild-type gene in the host cell. For example, the host cell can be contacted with an antisense nucleic acid or a small interfering RNA (siRNA) that inhibits expression of the corresponding wild-type GTPase but not the mutant GTPase. Nucleic acids designed for this purpose are available, for example, from GENSET (Boulder Colo.) and Qiagen (Valencia, Calif.); see also McManus and Sharp (2002) “Gene silencing in mammals by small interfering RNAs” Nature Rev. Gen. 3:737-747; Fire et al (1998) “Potent and specific genetic interference by double-stranded RNA in Caenorhabditis elegans” Nature 391:806-811; and Tuschl et al. (1999) “Targeted mRNA degradation by double-stranded RNA in vitro” Genes Dev. 13:3191-3197).

The GTPase modulator(s) used in the various methods of the present invention include, but are not limited to, chemical structures based upon a guanine, guanosine, GMP, GDP, or GTP scaffold. Preferably, one or more of the purine ring positions (e.g., C-2, C-6, N-7 and/or N-9) are substituted with an alkyl or aromatic substituent, such as those described above. In some embodiments, the modulators are cell permeable. Optionally, any phosphate groups present on the modulator are provided in caged form. Alternatively, modulators can be delivered into cells as described, e.g., in Cool et al. (1999) “The Ras mutant D119N is both dominant negative and activated” Mol. Cell. Biol. 19:6297-6305; Holt et al. (2002) “A chemical-genetic strategy implicates myosin-1c in adaptation by hair cells” Cell 108:371-381.

### Determining GTPase Function

In one aspect, the present invention provides methods of determining the function of a selected GTPase. The methods include the steps of a) expressing at least one mutant GTPase in one or more host cells; b) contacting the mutant GTPase with at least one GTPase modulator that binds to the mutant GTPase but does not substantially modulate a corresponding wild-type GTPase; and c) detecting at least one result of applying the GTPase modulator to the cell, thereby determining the function of the GTPase.

The methods of the present invention can be used to determine the function of any selected GTPase-encoding sequence. In one embodiment, the GTPase selected for modification is a Ras GTPase. Preferably, the mutant GTPase employed in the methods of the present invention has a non-native amino acid residue substituted at one or more positions corresponding to L19, F28, N116, K117 or T144 in the H-Ras sequence. Optionally, the selected amino acid (or amino acids) are independently substituted with an alanine, a glycine, or a cysteine residue. In some embodiments, the GTPase modulator includes an affinity label, such as a maleimide or acrylamide moiety, which can optionally react to form a covalent linkage between the modulator and the GTPase, potentially altering, e.g., reducing or inhibiting, the GTPase activity. In the protein embodiments employing a cysteine substitution, contacting the cysteine-containing mutant GTPase with a thiol-containing GTPase modulator can lead to formation of a covalent linkage between the GTPase modulator and the binding site cysteine residue, thereby altering the activity of the GTPase. Optionally, this covalent linkage leads to either irreversible inhibition or constitutive activation of the GTPase activity.

The method for determining the function of the GTPase includes the step of detecting at least one result of applying the GTPase modulator to the cell. A number of approaches can be taken to detect a biochemical or phenotypic result arising from the interaction between the GTPase and the modulator. In one embodiment of the methods, intracellular GTP and GMP levels are monitored in the presence and absence of the modulator. In another embodiment of the methods, cellular extracts are exposed to the modulator in the presence or absence of radiolabeled GDP; release of the radiolabeled component is indicative of competitive binding by the modulator. In another embodiment, the GTP-bound form of the GTPase is specifically isolated by affinity chromatography and quantitated to measure the amount of activated GTPase inside the cell. In yet another embodiment of the methods, protein translation activity is monitored.

Optionally, detection of a result of the modulator-GTPase interaction involves determining and/or analyzing one or more downstream response pathways affected by modulating the GTPase. GTPases are involved in a number of metabolic pathways, including, but not limited to, cell proliferation, apoptosis, cellular transport, cytoskeletal reorganization, vesicular trafficking, nucleo-cytoplasmic transport, and spindle formation. Several diseases, such as neurofibromatosis-1 (NF1) and X-linked mental retardation, also involve alterations in GTPase-mediated pathways. Furthermore, approximately 15% of all human cancers have been shown to involve alterations in GTPase activity. Methodologies for analyzing and/or monitoring components of these (and other) pathways are available in the art, and as such can be employed in the detection step of the methods of the present invention. A selected pathway can be monitored; alternatively, multiple pathways can be examined, e.g., using global expression analysis tools (e.g., polynucleotide or protein “chip,” e.g., gene chip, assays) or proteomics approaches such as 2D electrophoresis and mass spectrometry (see, for example U.S. Ser. No. 10/289,462 and international application PCT/US02/35607 to Brock et al., which describe methods and devices for reducing proteomics data complexity. Mechanism for detecting and/or monitoring these and other GTPase-mediated diseases can also be used for detecting at least one result of applying the GTPase modulator to the cell.

Optionally, a plurality of results (e.g., a number of different downstream biochemical responses) are monitored during the detection step. In one embodiment, the data regarding the downstream response pathways are collected and stored in at least one database within, e.g., a computer system. This information can be used, for example, to identify genes that are upregulated or downregulated in the presence of the GTPase modulator, or to generate a gene expression profile of the host cell (or series of host cells) in the presence and absence of the GTPase modulator.

As a further aspect of determining GTPase function, the methods described herein can be used to assess one or more biochemical effects, or one or more phenotypic effects, resulting from GTPase activity.

### Reducing Intrinsic GTPase Activity

In addition, the present invention also provides methods of reducing activity of a GTPase in a cell. The methods include, but are not limited to, the steps of a) introducing a mutant GTPase into the cell, wherein the mutant GTPase comprises a non-native amino acid at one or more amino acid positions that correspond to N116, T144 and L19 of H-Ras, which mutant GTPase binds to a GTPase modulator that alters the activity of the mutant GTPase but does not substantially affect the activity of a corresponding wild-type GTPase; and b) contacting the mutant GTPase with the GTPase modulator, thereby competing with the wild-type GTPase for binding to one or more cellular effector molecules and reducing the activity of the GTPase in the cell.

Unless otherwise interfered with, the host cell will express both the normal (wild-type) contingent of GTPases as well as the mutant GTPase. The mutant GTPase will be in competition with the wild-type GTPases for interaction effector molecules available in the cellular environment. Over expression of the mutant GTPase will reduced the availability of these components for the wild-type protein; however, if the mutant GTPase activity mirrors that of the wild-type GTPase, no cellular effect is seen (in the absence of a modulator). Addition of the modulator alters the activity of the mutant GTPase. While the orthogonal modulator will not directly interact with the wild-type GTPase, the available pool of effector molecules is tied up in the modulated mutant GTPase, thus effectively reducing the overall GTPase activity of the cell.

In another embodiment, the method further comprises the step of administering an antisense nucleic acid or an siRNA that inhibits expression of the corresponding wild-type GTPase but not the mutant GTPase. The presence of the antisense nucleic acid or siRNA effectively further reduces the GTPase activity in the cell.

### Methods for Determining GTPase Binding Proteins

In another aspect, the present invention provides methods of determining one or more GTPase binding proteins, or effector molecules, which interact with a selected GTPase protein. The methods include the steps of a) providing a mutant GTPase, which retains the effector specificity (binding properties) of a corresponding wild-type GTPase; b) contacting the mutant GTPase with at least one GTPase modulator that binds to the mutant GTPase but does not substantially inhibit or activate the corresponding wild-type GTPase; c) contacting the mutant GTPase complex including the orthogonal modulator with at least one GTPase binding protein or effector molecule; and d) detecting the at least one GTPase effector molecule, thereby determining one or more effector molecules that bind the GTPase. Detection can be performed, for example, by isolation and sequencing of a bound effector molecule.

For example, a mutant GTPase incorporating a sequence tag, such as a GST- or His-tagged mutant GTPase, can be expressed in vivo as described above, and then purified using ligand-coupled beads. To identify upstream and/or downstream effectors, the mutant GTPase remains attached to the beads. The beads are incubated with dialyzed whole cell lysate containing a modulator, such as compound 60 and/or compound 52 (e.g., at approximately 10 μM). The beads are washed with buffer containing MgCl₂ and 1 μM modulator. The beads are then divided into two (or more) aliquots, and incubated in a minimal amount (e.g., to disperse or suspend) of elution buffer or elution buffer containing EDTA and 100 μM GTP (inhibitor) or GDP (activator). The eluted samples are electrophoresed on separate two dimensional gels, and the gels are aligned and compared to identify proteins present only in the presence of either compound 60 or compound 52. Proteins identified following incubation with compound 60 are upstream effectors, whereas proteins identified following incubation with compound 52 are downstream effectors of the GTPase.

Because all GTPases are turned on and off using GTP and GDP, respectively, while the mutant GTPases (e.g., GTPases comprising a mutation at positions 19 and 116, relative to the Ras GTPase), can be turned off selectively using the modulators (e.g., compound 60, compound 52), the modulators of the invention can be used for the highly selective inhibition and/or activation of the mutant GTPase in the presence of all other GTPases. This is advantageous, since GTPases can have partially overlapping biological roles, and often share some upstream and downstream effectors. Additionally, the highly selective binding characteristics of the modulators (e.g., compound 60, compound 52) produce a concentration effect based on selective binding by the modulator followed by selective release induced, for example, by EDTA in the presence of GTP or GDP. This results in a greater than 1000 fold increase in the concentration of proteins that are potential effectors of the GTPase. Optionally, comparison of 2D gels obtained under different conditions provides a further enrichment by allowing differentiation between signal (proteins present in the presence of EDTA and GTP or GDP) and background noise.

### Determining Whether Test Compounds Modulate GTPase Activity

In yet another aspect, the present invention provides methods for assaying test compounds for GTPase modulatory activity. The methods include the steps of a) providing a mutant GTPase; b) contacting the GTPase with one or more members of a library of test compounds; and c) detecting a GTPase activity. Screening of compound libraries using one or more GTPase assays known to one of skill in the art. For example, GDP or GTP labeled with a radiolabel (³H or ³²P), biotin, fluorescein, or ethidium bromide can be included in the reaction mixture; release of the labeled GDP from the GTP-GTPase complex can be measured by, for example, thin layer chromatography, spectrophotometry, immunoprecipitation, or any of a number of other techniques known to one of skill in the art. See the protocols provided in, for example, Balch et al. (1995) “Small GTPases and Their Regulators”, Methods in Enzymology, volume 255, Academic Press.

Optionally, the step of providing the mutant GTPase protein includes partially purifying or isolating the protein, for example, by ammonium sulfate precipitation or immunoprecipitation. Alternatively, providing the mutant GTPase protein involves providing an intact host cell expressing the mutant GTPase (or a cellular extract thereof). The library of test compounds to be examined can include about 10, about 25, about 50, about 75, about 85, about 95, about 100, or more compounds. These compounds can be contacted with the GTPase individually or in subsets. Optionally, the methods further include the step of d) comparing a GTPase activity in the presence of a test compound with a GTPase activity in the absence of test compound.

### Treatment of GTPase-Mediated Diseases

GTPases have been shown to be involved in a number of important cellular processes. Alterations in GTPase activity (either by excessive signaling or reduced activity) can potentially lead to a number of disease states shown to have a GTPase-mediated component. For example, autoimmune diseases such as rheumatoid arthritis and lupus erythematosis result from the abnormal activation of leukocytes and/or lymphocytes, a process mediated by GTPase coupled receptors. Irregularities in GTPase activity can also potentially lead to abnormal cellular proliferation (via an excess of the signaling molecule cyclic AMP), various vesicular transport diseases, defects in protein trafficking. In addition, some microbial pathogens (such as Salmonella) target GTPase activity, e.g., to facilitate microbial invasion (see, for example, Progress in Mol. Subcell. Biol. (1999) 22: 183-199). Thus, GTPases play a pivotal role in the pathogenesis of a number of disease states.

The present invention provides methods for assessing therapeutic values of GTPase modulators. As a related aspect, the present invention also provides methods for treating GTPase-mediated diseases. The methods include the steps of a) providing a mutant GTPase to a cell; b) treating the cell with a GTPase modulator; and c) monitoring an effect of modulating a GTPase activity in the cell.

In one embodiment for assessing therapeutic values of a GTPase modulator, the GTPase is provided to a cell culture. The cell culture can then be treated with one or more putative modulators (or a library of modulators, e.g., inhibitors and/or activators). The cells (or cellular extracts thereof) are monitored for an effect resulting from exposure to the modulator: for example, cell permeability of the modulator compound, a modulatory activity, a downstream biochemical reaction, or a phenotypic effect. The detected effect can be used as a measure of therapeutic value of the modulator.

Alternatively, the GTPase can be provided to a cell in an animal, e.g., by gene therapy or other cell transformation procedures. The animal is then treated with a therapeutic quantity of a GTPase modulator, and a biochemical reaction or phenotypic effect detected (i.e., improvement in or compensation of a pathological condition arising from a GTPase-mediated biochemical pathway).

Activity or expression of a corresponding wild-type GTPase need not be reduced in order to see a remedial effect via therapeutic treatment with a mutant GTPase. This is often the case for methods targeted toward reducing GTPase activity in vivo as a therapeutic approach to a GTPase-mediated disease. In embodiments in which both the wild-type and mutant GTPase sequences are expressed, both protein pools are potentially competing for binding to the same regulatory and effector molecules. If the pool of mutant GTPase molecules is substantially larger than the wild-type pool (or if the modifications in protein structure also enhance effector binding), the majority of effector molecules will be associated with mutant constructs. Modulation of the mutant GTPases with specific modulators then reduces the overall GTPase activity in the cell, by forcing the wild-type GTPase proteins to compete with the modulated mutant GTPases.

### Enzyme Kinetic and Mechanistic Studies

The mutant GTPases and modulators of the present invention can also used in and/or characterized by classical enzymatic kinetic analysis (see, for example, Fersht Enzyme Structure and Mechanism (1985, W.H. Freeman and Company, New York), and references cited therein.

### Cell Systems

The present invention also provides cell systems and host cells incorporating one or more of the mutant GTPases of the present invention. Cells for use in the present invention include prokaryotic and eukaryotic cells. In a preferred embodiment, the cell system comprises mammalian cells. Optionally, the host cell is selected from the group consisting of fibroblasts, myeloid leukemia cells, and Raji cells.

As noted above, the cell system can be transformed to express the mutant GTPase sequence by any of a number of techniques known in the art, including, but not limited to, electroporation, microinjection, lipofection, particle bombardment, or polyethylene glycol-mediated transformation using a GTPase-containing expression vector. The expression vector (e.g., plasmid, cosmid, expression cassette, etc.) used to generate the cell system will depend in part upon the cell system selected to be transformed. Optionally, the expression vector includes a mechanism for regulating expression of the mutant GTPase, such as inducible genetic regulatory sequences. In some embodiments, the expression vector also includes a mechanism for stably transforming the cell system, e.g., recombinases.

The host cell has a nucleic acid sequence for expressing a mutant GTPase which binds to a “mutant GTPase-selective” modulator (i.e., a GTPase modulator that alters the activity of the mutant GTPase but does not substantially affect the activity of a corresponding wild-type GTPase). In a preferred embodiment, the amino acid sequence of the mutant GTPase has a non-native amino acid at one or more amino acid positions that correspond to L19, F28, N116, K117, or T144 of H-Ras.

In one embodiment of the present invention, the host cell does not express the wild-type GTPase that corresponds to the mutant GTPase. This can be achieved, for example, by disrupting the expression of the wild-type GTPase using an antisense RNA or a siRNA.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Analysis of Engineered GTPases Having a Single Amino Acid Substitution

To develop specific modulators for GTPases using an “engineered receptor/orthogonal modulator” approach, eight Ras mutants were generated having a single amino acid substitution (either alanine or glycine) in one of four positions: lysine-19 (L19), phenylalanine-28 (F28), asparagine-116 (N116) or lysine-117 (K117). Throughout these examples, wild-type H-Ras was employed, the sequence of which is provided in SEQ ID No. 1 (amino acid sequence) and SEQ ID No. 18 (nucleic acid sequence). However, any GTPase can be similarly modified, and the corresponding positions determined, e.g., using BLAST, as indicated above. The single-mutation Ras sequences are presented in amino acid SEQ ID NOS. 2 to 9 and nucleic acid SEQ ID NOS. 19 to 26, respectively.

These mutations resulted in the structural “truncation” of amino acid sidechains that are in close contact to the purine ring of the GTP substrate. Different pockets were created near the nucleotide binding site, based upon the single mutation generated. The effect of the modification in substrate pocket size were assessed by assaying GTPase activity of the mutant proteins in the presence of ³H-labeled GDP and unlabeled modulators of the present invention.

A number of GTPase modulators were synthesized using a guanine (purine ring) scaffold modified at the C-6, N-7 and/or N-9 positions (see FIG. 3). For the structures having a modification at position 7 of the purine ring, the nitrogen atom was replaced with a carbon atom at position 7, while the carbon atom present at position 8 was optionally replaced with a nitrogen atom. These modulators demonstrated approximately 10% or less inhibition of the mutant GTPases in the presence of radiolabeled GDP (data not shown).

TABLE 2
Structures of Guanine-based Modulators

| Cpd | GNF | | N-7 (or C-7) | |
|---|---|---|---|---|
| No. | Ref. | C-6 moiety | moiety | N-9 moiety |
| 1 | GB1 | O-benzyl | n/a | benzyl |
| 2 | GB2 | O—(CH₂)₂-phenyl | n/a | benzyl |
| 3 | GB3 | NH-benzyl | n/a | benzyl |
| 4 | GB4 | NH—(CH₂)₂-phenyl | n/a | CH₂ C(CH₃)₃ |
| 5 | GB5 | O—CH₂ C(CH₃)₃ | n/a | benzyl |
| 6 | GB6 | NH—(CH₂)₂ phenyl | n/a | benzyl |
| 7 | GB7 | O-isopropyl | n/a | benzyl |
| 8 | GB8 | O-benzyl | n/a | CH₂ C(CH₃)₃ |
| 9 | GB9 | OCH₂ naphthyl | n/a | benzyl |
| | | (linked via | | |
| | | the naphthyl C1) | | |
| 10 | GB10 | O—(CH₂)₃ cyclohexyl | n/a | benzyl |
| 11 | GB11 | O—(CH₂)₂ cyclohexyl | n/a | benzyl |
| 12 | GB12 | NH-isopropyl | n/a | benzyl |
| 13 | GB13 | NH-isobutyl | n/a | CH₂ C(CH₃)₃ |
| 14 | GB14 | O—CH₂ cyclohexyl | n/a | benzyl |
| 15 | GB15 | O—(CH₂)₂ naphthyl | n/a | benzyl |
| | | (linked via | | |
| | | the naphthyl C2) | | |
| 16 | GA1 | ketone | phenyl | benzyl |
| 17 | GA2 | ketone | CH₂ naphthyl | benzyl |
| | | | (linked via the | |
| | | | naphthyl C1) | |
| 18 | GA3 | ketone | cyclohexyl | benzyl |
| 19 | GA4 | ketone | CH₂ naphthyl | CH₂ C(CH₃)₃ |
| | | | (linked via the | |
| | | | naphthyl C1) | |

### Example 2

### Analysis of Engineered GTPases Having Two Amino Acid Substitutions

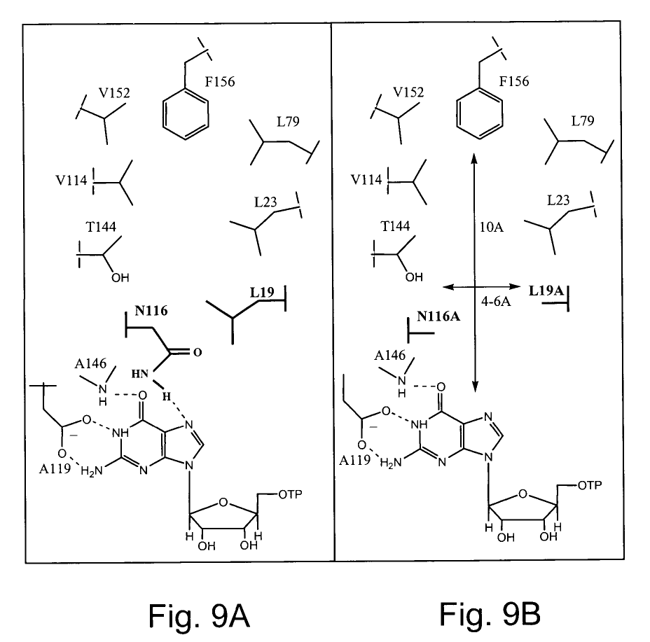

Typically, the sidechains of N116 and L19 act as gatekeepers to the GTP binding site, since they are positioned at the back side of the pocket (see Figure BBB, panel A) and thereby delineate the binding interaction between wild-type Ras and GTP. As noted previously, mutation of these amino acid positions and alteration of the steric hindrance at the back of the pocket would affect modulator affinity. Further enlargement of the binding pocket to create a cavity large enough such that synthetic modulator having a bulky substituent would be able to enter, and potentially make hydrophobic interactions, was also considered in the present invention. For example, mutation of N116 to alanine (N116A) or glycine (N116G) concomitant with mutation of L19 to alanine (L19A) or glycine (L19G) generated approximately 4 to 6 angstroms of space between the two residues and an accessible hydrophobic pocket that was approximately 10 angstroms deep. (See FIG. 9, panel B).

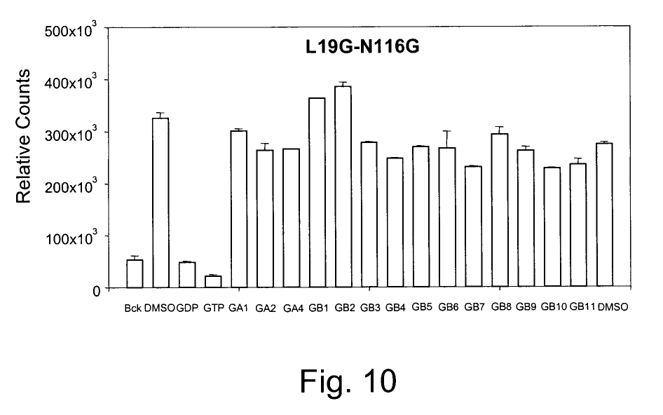

Several Ras mutants were engineered with double mutations as represented in SEQ ID No. 10 to 15 (amino acid sequences) and 27 to 32 (corresponding nucleic acid sequences). These mutants were expressed and tested against the compounds provided in Table 2, as well as the control compounds GTP, GDP, and DMSO. FIG. 10 graphically illustrates the modulation of one of the double mutants, L19G-N116G (SEQ ID No. 13). The assay performed in the absence of mutant GTPase provided a negative control (Bck, or background counts).

The ³H-labeled GDP exhibited strong binding to the L19G-N116G mutant GTPase, as shown by the high counts of the DMSO sample (in which the radiolabeled GDP has not been displaced from the protein). It should be noted that the actual counts do not have any particular significance in determining modulatory activity. The relative intensity is the important factor; comparisons can only be made within each particular evaluation. The term “relative counts” refer to the counts for the various GTPase modulators graphed as a percentage of the control counts when the control is given the value of 100%. Anything below 50% of the Control's Counts was considered significant inhibition.

While a number of the modulator compounds demonstrated some minor inhibitory effect against the double mutant, the binding affinities were not great enough to substantially compete for the binding site and thereby induce the release of the labeled GDP substrate. One reason for the weaker binding affinities might be the absence of the phosphate groups present in GTP and GDP. In order to increase the binding affinity, additional modulator compounds based upon phosphorylated scaffolds were prepared.

### Example 3

### Analysis of Modulations Based Upon the GTP-Scaffold

A second set of GTPase modulators were synthesized using a guanosine-triphosphate scaffold modified at the C-6 and/or N-7(or C-7) positions (position N-9 being attached to the phosphorylated ribose moiety; see FIG. 4). The C-7 moieties described were linked to C-7 through a two methylene unit linker.

TABLE 3
Structures of GTP-based Modulators

| Com- | | N-7 | |
|---|---|---|---|
| pound | | (or C-7) | |
| No. | C-6 moiety | moiety | N-9 moiety |
| 39 | —NH—CH₂C₆H₅ | n/a | ribose-triphosphate |
| 40 | —O—CH₂CH(CH₃)₂ | n/a | ribose-triphosphate |
| 41 | —NH—(CH₂)₂C₆H₅ | n/a | ribose-triphosphate |
| 42 | —NHCH₂CH (CH₃)₂ | n/a | ribose-triphosphate |
| 43 | —O—CH₂CH═CH₂ | n/a | ribose-triphosphate |
| 44 | —O—CH₃ | n/a | ribose-triphosphate |
| 45 | —SH | n/a | ribose-triphosphate |
| 49 | —O—CH₂CH₂ (3,4- | n/a | ribose-triphosphate |
| | dimethyl)maleimide | | |
| 50 | —O—CH₂CH₂CH₂-(3,4- | n/a | ribose-triphosphate |
| | dimethyl)maleimide | | |
| 51 | —OCH₂CH₂NHCOCH═CH₂ | n/a | ribose-triphosphate |

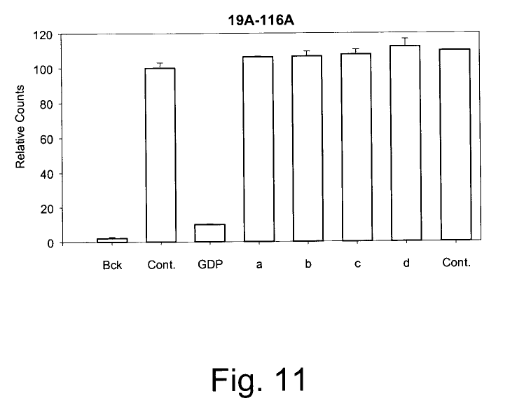

FIG. 11 graphically illustrates the inhibition of double mutant L19A-N116A (SEQ ID No. 11) by various C-6 substituted GTP moieties (compounds 39 through 42, presented as a-d in the figure), as compared to the negative control (nothing added to displace the radiolabeled GDP), positive control (cold GDP), and background count (no GTPase enzyme added). The GTPase modulator compounds 39-42 showed very little inhibitory effect on the activity of the L19A-N116A double mutant. The data are displayed as relative counts, with the negative control (Cont.) set to a maximum measurement of 100%, and the counts for inhibitor results presented as a percentage relative to the control.

### Example 3

### Part 2: Analysis of Modulations Based Upon the GTP-Scaffold

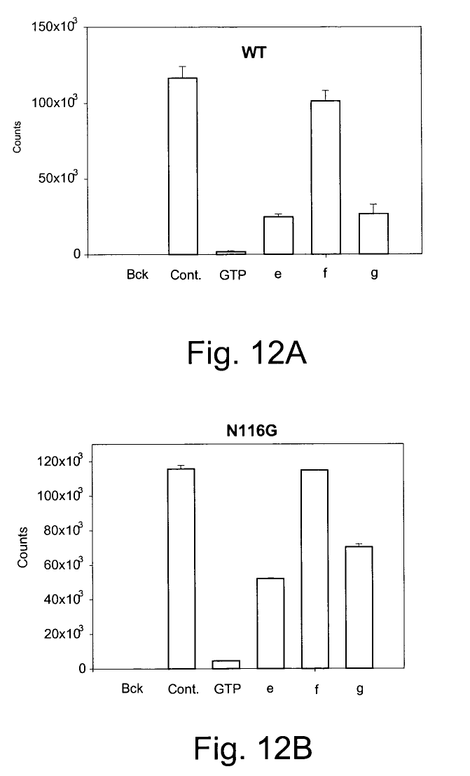

FIG. 12 provides the inhibitor assay results for compounds 43 through 45 (e.g, respectively) as compared to the positive control GTP, for the wild-type Ras protein (panel A) and the single mutant N116G (SEQ ID No. 7). The test compounds 43 and 45, which are structurally based upon a C-6 modified GTP scaffold, are shown to significantly inhibit the mutant GTPase, as reflected in the relative decrease in radiolabel remaining bound to the protein. Thus, substitution of the N116 residue with a glycine appears to be a favorable mutation leading to expansion of the GTP binding pocket.

Since the modulators also displayed significant inhibition of the wild-type Ras, it was concluded that the C-6 modified to the GTP-based modulator scaffold does not provide as desired a selectivity for the N116G mutated enzyme. However, the combination of this mutation with other types of modulators would probably result in improved inhibitory effects.

### Example 4

### Analysis of Modulations Based Upon the GDP-Scaffold

C-7 substituted GDP-based modulators were synthesized and tested for inhibitory activity versus various mutant GTPases. As noted previously, the nitrogen atom normally present at position 7 of the purine ring has been replaced by a carbon atom. In addition, for most of the modulator compounds tested, the moiety present in guanosine at C-6 is an hydroxyl group, thereby allowing ketone formation at C-6. The C-7 moieties described were linked to C-7 through a two methylene unit linker.

TABLE 4
Structures of GDP-based Modulators

| | | | C-7 moiety | |
|---|---|---|---|---|
| No. | compound | C-6 moiety | (—CH₂CH₂R) | N-9 moiety |
| 57 | a | ketone | —CH(CH₃)₂ | ribose-diphosphate |
| 58 | b | ketone | —C(CH₃)₃ | ribose-diphosphate |
| 59 | c | ketone | —C₆H₁₁ | ribose-diphosphate |
| 60 | d | ketone | —C₆H₅ | ribose-diphosphate |
| 61 | e | ketone | —C₆H₄ p-F | ribose-diphosph ate |
| 62 | f | ketone | —CH₂C₆H₅ | ribose-diphosphate |
| 63 | g | ketone | -(2-napthyl) | ribose-diphosphate |
| 64 | h | —NH₂ (deaza | —C₆H₅ | ribose-diphosphate |
| | | adenosine | | |
| | | scaffold) | | |

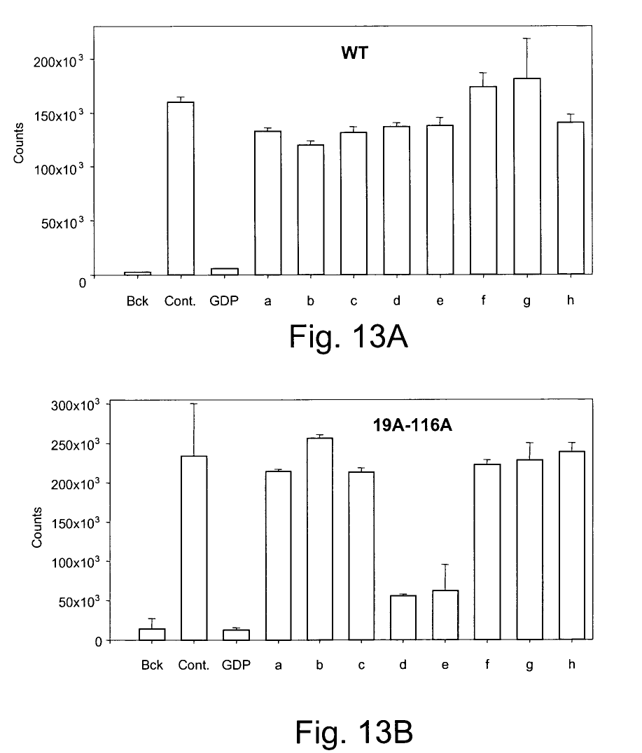

FIG. 13 graphically illustrates the inhibitory effect of the C-7 substituted GDP-scaffold modulators on wild-type Ras (panel A) and on the double mutant L19A-N116A. As shown in the figures, the C-7 substituted GDPs showed very little inhibition of the wild-type GTPase. However, at least two of the modulators (d and e, compounds 60 and 61) showed strong inhibition of the L19A-116A mutant. The inhibitory effect was in the range of approximately 80-95%. Thus, modification at the N-7 position precluded binding of the modulator to the wild-type GTPase protein and displacement of the radiolabeled GDP.

### Example 5

### Analysis of Modulations Based Upon the GMP and Guanosine-Scaffolds

In addition, modulators based upon a modified GMP scaffold or a guanosine ring were also synthesized and tested for GTPase inhibitory activity. By reducing the number of phosphates on the scaffold, these modulators are potentially more cell permeable than the more phosphorylated modulators.

TABLE 5
Structures of GMP-based Modulators

| No. | C-6 moiety | C-7 moiety | N-9 moiety |
|---|---|---|---|
| 66 | ketone | OCH₂CH₂NHCOCH₂CH₂- | ribose-monophosphate |
| | | maleimide | |
| 80 | —O— | n/a | ribose-monophosphate |
| | CH₂CH₂NHCOCH═CH₂ | | |
| 81 | —O— | n/a | ribose-monophosphate |
| | CH₂CH₂NHCOCH₂CH₂- | | |
| | maleimide | | |
| 82 | —SH | n/a | ribose-monophosphate |
| 83 | —O—CH₂CH₂-(3- | n/a | ribose-monophosphate |
| | methyl)maleimide | | |
| 84 | —O—CH₂CH₂-(3,4- | n/a | ribose-monophosphate |
| | dimethyl)maleimide | | |
| 85 | —O—CH₂CH₂CH₂-(3,4- | n/a | ribose-monophosphate |
| | dimethyl)maleimide | | |

TABLE 6
Structures of Guanosine-based Modulators

| No. | C-6 moiety | N-7 moiety | N-9 moiety |
|---|---|---|---|
| 21 | —O—CH₂NHCOCH₂Br | n/a | ribose |
| 22 | —O— | n/a | ribose |
| | CH₂NHCOCH₂CH₂- | | |
| | maleimide | | |
| 23 | —O—CH₂NHCOCH═CH₂ | n/a | ribose |
| 24 | —O—CH₂NHCOC₆H₄- | n/a | ribose |
| | pSO₂F | | |
| 25 | | | |
| 26 | —SH | n/a | ribose |
| 27 | —O—(CH₂)₂SH | n/a | ribose |
| 28 | —O—(CH₂)₃SH | n/a | ribose |
| 29 | —O—(CH₂)₄SH | n/a | ribose |
| 36 | —O—CH₂CH₂-(3- | n/a | ribose |
| | methyl)maleimide | | |
| 37 | —O—CH₂CH₂-(3,4- | n/a | ribose |
| | dimethyl)maleimide | | |
| 38 | O—CH₂CH₂CH₂-(3,4- | n/a | ribose |
| | dimethyl)maleimide | | |

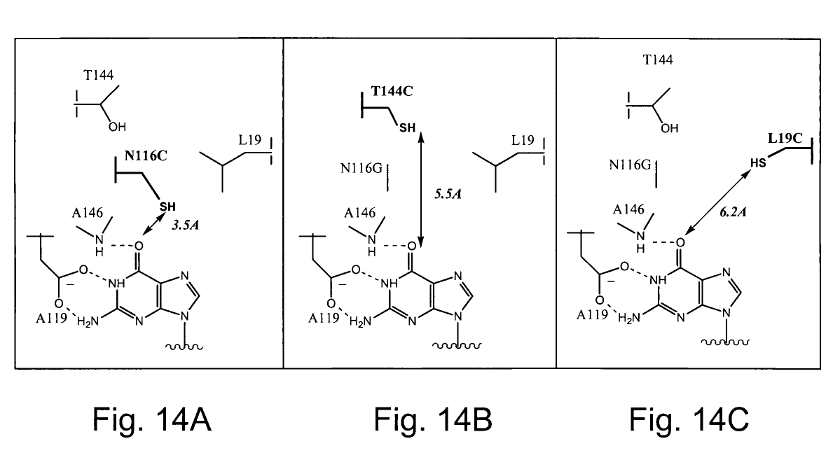

FIG. 14 is a schematic representation of the position of three residues in the GTP binding site (L19, N116 and T144) with respect to the purine ring of a substrate structure. Panels A, B and C depicts the distances generated between the purine ring ketone at C-6 and a thiol group sidechain upon substitution with the non-native amino acid cysteine at residue N116, T144 and L19, respectively. Modulators based upon a guanosine or guanosine monophosphate (GMP) scaffold were synthesized for analysis; certain modulators included an electrophilic group at the C-6 or N-7 position. The optional presence of either an electrophilic or a thiol group makes a covalent protein-modulator interaction possible.

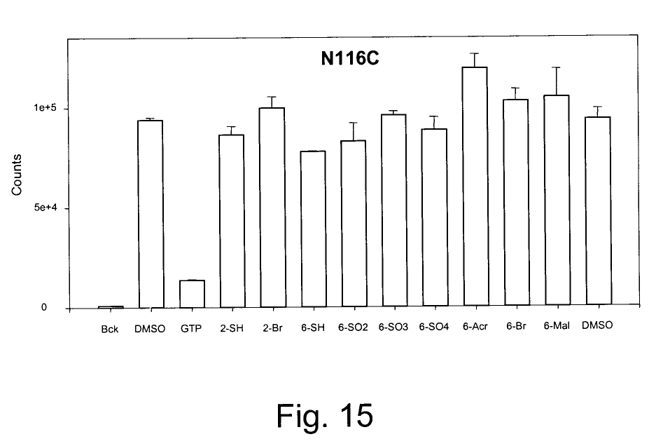

FIG. 15 provides a bar chart depicting the ability of various guanosine-based modulators to displace radiolabeled GDP from the GTPase mutant N116C. The lack of inhibition observed suggested that introduction of a monophosphate group on the ribose moiety might be beneficial since GMP is known to have higher affinity than guanosine for wild type GTPases.

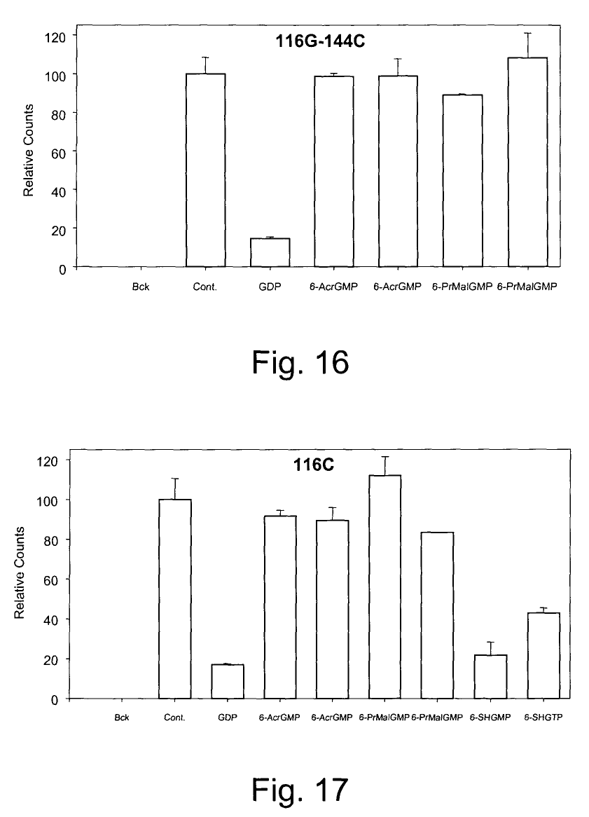

FIGS. 16 and 17 graphically illustrates the inhibitory effect of the C-6 or N-7 substituted GMP-scaffold modulators on the double mutant N116G-T114C and the mutant N116C, respectively. As shown in FIG. 17, compound 82 (6-SHGMP) having a thiol group positioned at C-6 showed strong inhibition of the N116C mutant. The inhibitory effect was in the range of approximately 50-80%.

### Example 6

### Signal Transmission Inhibition and Activation

GTPases cycle between GDP- and GTP-bound conformations, as shown in FIG. 32. Upstream signals determine the amount of GTP-bound enzyme that can bind to effectors and thus modulate the “switch” transmitting the signals downstream. The cyclical nature of GTP/GDP binding provides the basis for the “guanine nucleotide exchange assay” (FIG. 8) used to evaluate binding specificity and inhibitory or activating activities of potential modulators.

The GTPase modulators of the present invention were screened against wild-type and/or mutant GTPases for their effectiveness as GTPase inhibitors. Ras inhibitor assays were performed to test the activity of the synthesized modulators against various wild-type and/or mutant GTPases of interest, and/or to compare the different modulators based upon the relative observed level of inhibition.

For example, purified mutant Ras (19A-116A) or wild type H-Ras protein (0.5 to 20 μg) was incubated in 40 μL of GTP exchange buffer (50 mM Tris-HCl pH 7.5, 50 mM NaCl, 5 mM EDTA, 1 mM DTT, 1 mg/mL BSA) containing either DMSO, GTP (100 μM) or a modulator (100 μM) for 30 min at 25° C. This pre-incubation was followed by the additions of 2 μCi ³H-GDP (NEN, #NET966) and, 10 min later, 25 mM MgCl₂. After another 5 min of incubation, the reaction mixture was applied to a nitrocellulose filter (Millipore, #HAWP02500) pre-wetted with wash buffer (50 mM Tris-HCl pH 7.5, 10 mM MgCl₂), the filter was then washed using 10 mL of wash buffer. Filters were dried and the bound radioactivity was measured by scintillation counting (Beckman, #LS6500).

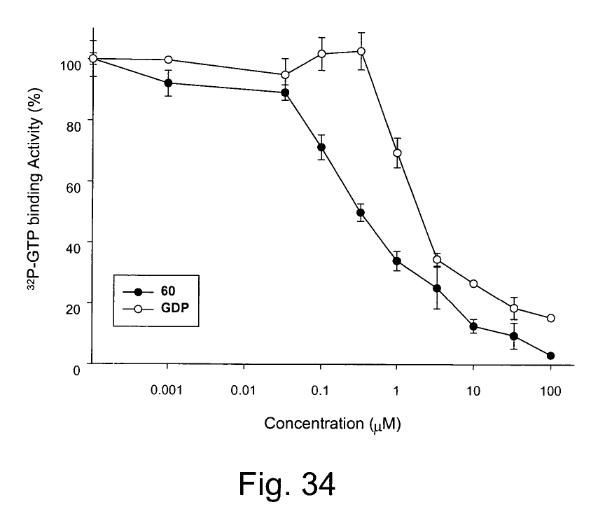

Cold GDP was used as a positive control. Since the positive control naturally replaces the radioactive GDP in both the wild-type and mutant GTPase proteins, significant drops in radiolabeling are observed. Water was used as a negative control. All of the measurements are in comparison to a background radioactivity count, such that the level of inhibition for a given compound was determined by the number of counts in comparison to the control. Thus, a high radioactivity count is indicative of a high level of binding between the mutant GTPase and the radioactive GDP. Potent GTPase modulators, e.g., Compound 52 (activator) and Compound 60 (inhibitor) displaced the radioactive GDP, and lowered the remaining radioactivity count as shown in FIG. 34A. For example, Compound 60 was found to be 5 times more potent than GDP at displacing GTP from the mutant GTPase protein (as measured by determining the IC₅₀ for Compound 60 (300 nM) and GDP (approximately 1.5 μM), respectively, as shown in FIG. 34. The IC₅₀ is the concentration of “inhibitor” at which 50% inhibition of GTP binding is observed. In contrast, Compound 60 does not inhibit wild-type H-Ras at a concentration of 100 μM.

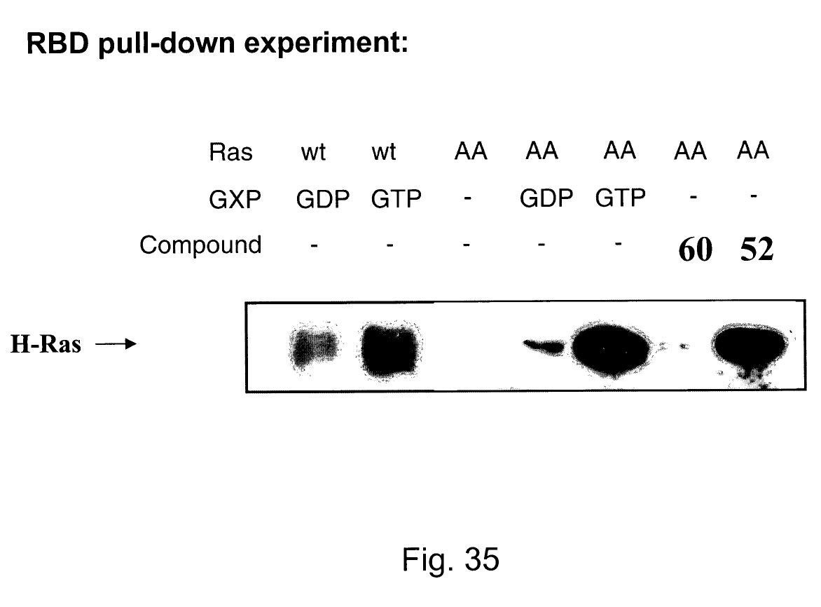

The ability of the mutant enzyme (19A-116A) to bind to a known effector of H-Ras was confirmed. Using the Ras binding domain (RBD) of the Raf kinase, the ability of the mutant enzyme to undergo a conformational change upon GTP binding, and, thus, to bind the effector was determined. In brief, purified mutant or wild-type proteins (150 ng) were incubated in 1 mL of loading buffer (100 mM Tris-HCl pH 7.5, 2 mM EDTA, 1 mM DTT, 1 mg/mL BSA) in the presence of 10 mM of GDP, GTP, 7 d or both GTP and 7 d for 15 min at 25° C. for loading the protein with the appropriate nucleotides. Following loading, MgCl₂ (20 mM) was added along with GST-RBD (1.2 mg), comprising Raf kinase amino acids 53-131, bound to glutathione sepharose beads. The resulting suspensions were incubated with rotation for 30 min at 4° C. The beads were then washed three times with 1 mL of washing buffer (50 mM Tris-HCl pH 7.5, 50 mM NaCl, 20 mM MgCl₂, 1 mM DTT) to remove unbound H-Ras proteins. The beads were boiled in SDS loading buffer, and the samples were separated by electrophoresis. Following transfer, the PVDF membrane was probed with anti-H-Ras and the amount of H-Ras bound to the RBD glutathione sepharose beads was visualized using anti-rabbit HRP in conjunction with WestPico (Pierce). As shown in FIG. 35, Compound 60 binds to the mutant enzyme and prevents effector binding, thus, inhibiting signal transmission. In contrast, Compound 52 binds to the mutant enzyme and prompts effector binding, activating signal transduction.

### Example 7

### Applicability to Other GTPases

To confirm the general applicability of the methods and compositions of the present invention in the context of GTPases other than H-Ras, alanine substitutions at amino acids corresponding to positions 19 and 116 of Ras were introduced into the Rap1B GTPase. As shown in the alignment of Table 1, the identical amino acid residues, i.e., leucine at position 19, and asparagine at position 116, are present in Rap1B.

The mutant Rap1B protein construct was made as described above with respect to the Ras mutant proteins. All mutant proteins were made in the pGEX-4T bacterial expression system using the Quickchange protocol (Qiagen) according to the manufacturer's instructions. Mutant Rap1B cDNAs were transformed individually in BL21-Gold competent cells (Stratagene) according to the manufacturer's instructions. An overnight culture of BL21-Gold cells containing one of the GST-Rap1B plasmids described above was diluted 1:50 in Superbroth medium (Q-Biogene) supplemented with 50 mg/L carbenicillin. After it had grown to an OD=0.6, synthesis of the GST-Rap1B protein was induced by the addition of IPTG (50 mM) and by further incubation at 30° C. for 12 h. After centrifugation at 3,500 g for 15 min at 4° C., the cell pellet was frozen at −80° C. for 1 h. The bacterial cell pellet was then resuspended in lysis buffer (20 mM HEPES pH 7.5, 75 mM KCl, 25 mM MgCl2, 5 mM DTT, 0.1 mM EDTA, 0.05% Triton X-100, 5 mM Benzamidine, 10 mg/L Aprotinin, 10 mg/L Antipain, 10 mg/L Pepstatin, 10 mg/L Leupeptin and 1 mM PMSF), chilled on ice and sonicated (Sonics, Vibracell) for 60 sec. The resulting lysate was centrifuged at 30,000 g for 30 min at 4° C., and the supernatant was added to glutathione sepharose beads (Pharmacia) for a 30 min incubation at 4° C. on a rotating wheel. The beads were washed with lysis buffer once, and then with wash buffer (50 mM Tris-HCl pH 7.5, 50 mM NaCl, 1 mM DTT) three times. The protein was eluted using 10 mM glutathione in wash buffer, concentrated and dialysed overnight against storage buffer (50 mM Tris-HCl pH 7.5, 50 mM NaCl, 0.2 mM DTT, 0.1 mM EDTA). Protein concentration was determined by a Bradford assay and the protein purity was assessed by gel electrophoresis.

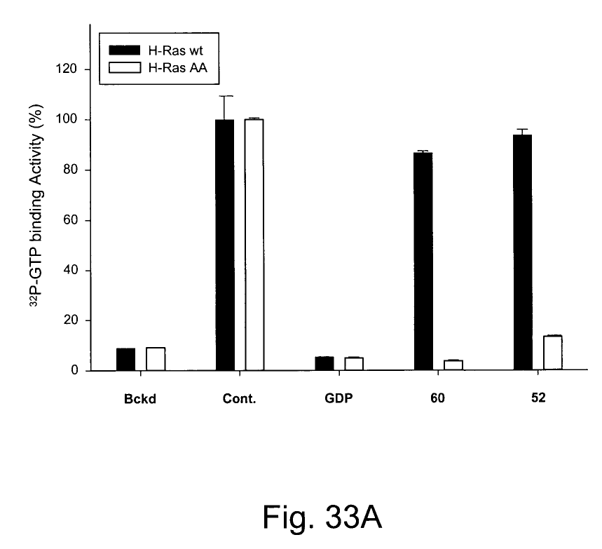

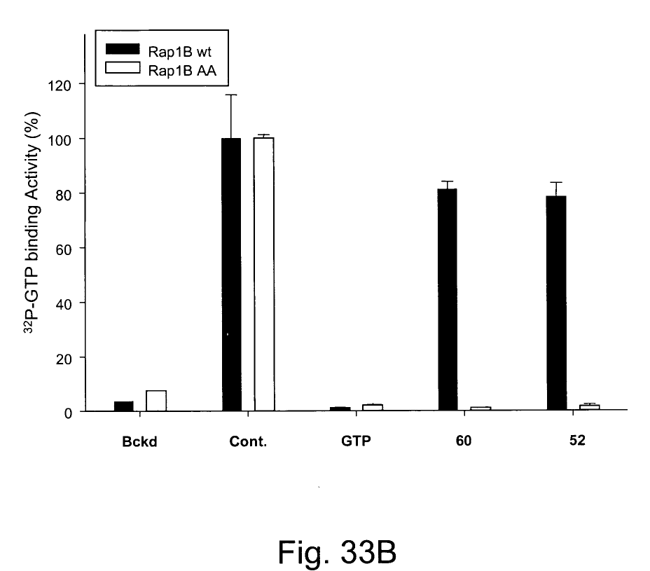

Both compounds 52 and 60 were found to bind tightly to the mutant Rap1B enzyme while negligably affecting the wild-type enzyme, as shown in FIG. 33B. These results demonstrate selective inhibition of the mutant Rap1B enzyme by modulators that similarly inhibit mutant Ras proteins, thus, indicating transferability of the methods and compositions of the present invention across members of the GTPase superfamily.

### Example 8

### Molecular Modeling of Modular Binding

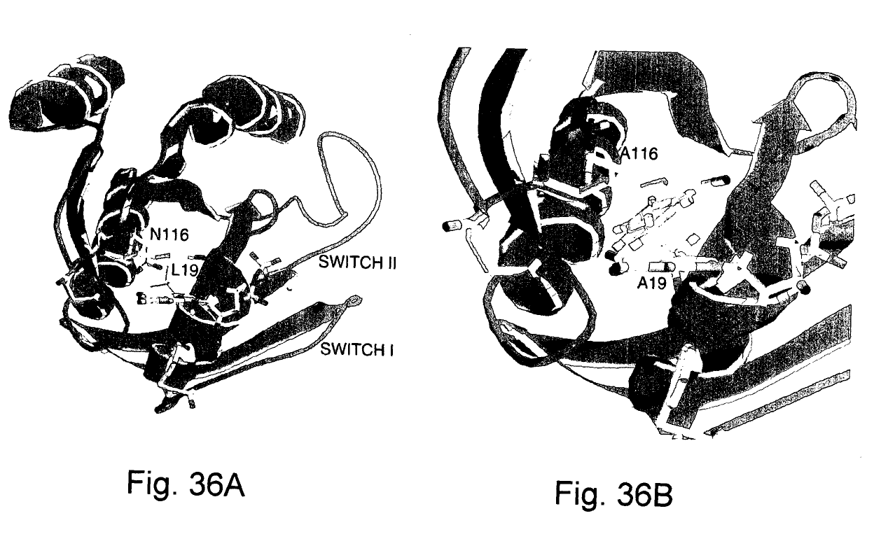

Using PDB entry 1Q21 as a template, docking of compound 60 to H-Ras L19A-N116A was accomplished using MOE. Briefly, amino acids 19 and 116 were mutated to alanine residues using MOE software. The resulting mutant GTPase bound to GDP was then energy minimized. The conformation of the mutant GTPase bound to compound 60 was then determined by fixing all atoms of the protein complex except for those part of the added 1-ethylphenyl unit. Random conformational searches revealed only one possible conformation for the phenyl group inside the substrate binding pocket of the mutant GTPase. This confirmation was further energy minimized and the output was visualized using a ray tracer. FIG. 36A illustrates a three dimensional model of the wild-type H-Ras GTPase-GDP complex. FIG. 36B provides a view of the (L19A-N116A) H-Ras GTPase-compound 60 complex.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. An orthogonal modulator of a mutant GTPase, which orthogonal modulator modulates an activity of a mutant GTPase but does not substantially modulate an activity of a corresponding wild-type GTPase.

2. The othogonal GTPase modulator of claim 1, wherein the GTPase modulator comprises a guanine ring modified at one or more of a C-6, N-7 and/or N-9 position.

3. The othogonal GTPase modulator of claim 1, wherein the GTPase modulator comprises a guanosine modified at a C-2, C-6 and/or N-7 position.

4. The othogonal GTPase modulator of claim 1, wherein the GTPase modulator comprises a structure selected from the group consisting of: a guanosine triphosphate modified at a C-6 and/or N-7 position; a guanosine diphosphate modified at a C-6 and/or N-7 position; and a guanosine monophosphate modified at a C-6 and/or N-7 position.

5. The othogonal GTPase modulator of claim 1, wherein the GTPase modulator comprises a cell permeable compound.

6. The othagonal GTPase modulator of claim 1, wherein the GTPase modulator comprises:
a) a sulfhydryl group that forms a disulfide linkage with a sulfhydryl group on N116C of the mutant GTPase, or
b) an electrophilic group that forms a covalent bond with a sulfhydryl group on N116C of the mutant GTPase.

7. The othogonal GTPase modulator of claim 1, wherein the GTPase modulator comprises:
a) a sulfhydryl group that forms a disulfide linkage with a sulfhydryl group on T144C, or
b) an electrophilic group that forms a covalent bond with a sulfhydryl group on T144C.

8. The othogonal GTPase modulator of claim 1, wherein the GTPase modulator comprises:
a) a sulfhydryl group that forms a disulfide linkage with a sulfhydryl group on L19C, or
b) an electrophilic group that forms a covalent bond with a sulfhydryl group on L19C.

9. The othogonal GTPase modulator of claim 1, wherein the GTPase modulator comprises the structure: 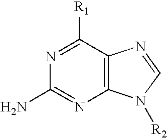
wherein R1 is selected from the group consisting of: O-benzyl, O—(CH₂)₂phenyl, NH-benzyl, NH—(CH₂)₂phenyl, O—CH₂tert-butyl, O-isopropyl, O—(CH₂)₂naphthyl, O—(CH₂)₃cyclohexyl, O—(CH₂)₂cyclohexyl, O—CH₂ cyclohexyl, and N-isobutyl; and
wherein R2 comprises a benzyl group or a methyl-tert-butyl group.

10. The othogonal GTPase modulator of claim 1, wherein the GTPase modulator comprises the structure: 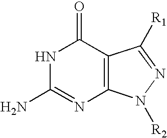
wherein R1 is selected from the group consisting of phenyl, CH₂-naphthyl, and cyclohexyl; and
wherein R2 is selected from the group consisting of benzyl and methyl-tert-butyl.

11. The othogonal GTPase modulator of claim 1, wherein the GTPase modulator comprises the structure: 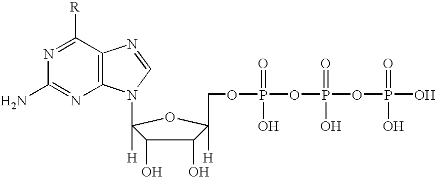
wherein R is selected from the group consisting of NH-benzyl, O-isobutyl, NH-(2-phenyl)ethyl, NH-isobutyl, O-2-propene, O-methyl, and thiol.

12. The othogonal GTPase modulator of claim 1, wherein the GTPase modulator comprises the structure: 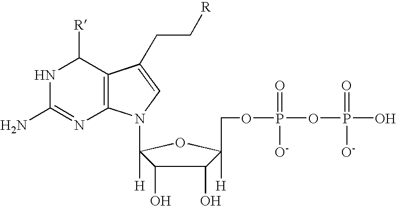
wherein R is selected from the group consisting of: isopropyl, tert-butyl, cyclohexyl, phenyl, 4-fluorophenyl, benzyl, and 2-naphthyl; and
wherein R′ comprises a ketone or NH₂.

13. The othogonal GTPase modulator of claim 1, wherein the GTPase modulator comprises the structure: 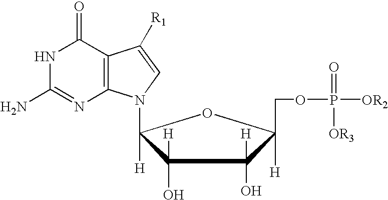
wherein R1 comprises thiol, CH₂SH, —CH₂CH₂SH, —CH₂CH₂CH₂SH, —CH₂CH₂CH₂CH₂SH, —CH₂CH₂NHCOCH₂CH₂—N-maleimide, —CH₂CH₂NHCOCH═CH₂, —CH₂CH₂NHCO-(4-fluorosulfonyl)phenyl, or 4-fluorophenyl; and
wherein R2 and R3 are independently selected from the group consisting of —OCH₂OCH₂CH₃, —OCH₂CH₂SCOCH₃, and —OCH₂CH₂CH₂CH₂CH₂SCOCH₃.

14. The othogonal GTPase modulator of claim 1, wherein the GTPase modulator comprises the structure: 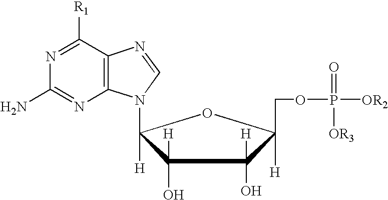
wherein R1 comprises thiol, —OCH₂CH₂SH, —OCH₂CH₂CH₂SH, —OCH₂CH₂CH₂CH₂SH, —OCH₂CH₂NHCOCH₂CH₂—N-maleimide, —OCH₂CH₂NHCOCH═CH₂, —OCH₂CH₂NHCO-(4-fluorosulfonyl)phenyl, OCH₂CH₂-(3-methyl)maleimide, —OCH₂CH₂-(3,4-dimethyl)maleimide, or OCH₂CH₂CH₂-(3,4-dimethyl)maleimide; and
wherein R2 and R3 are independently selected from the group consisting of —OCH₂OCH₂CH₃, —OCH₂CH₂SCOCH₃, and —OCH₂CH₂CH₂CH₂CH₂SCOCH₃.

15. The othogonal GTPase modulator of claim 1, wherein the GTPase modulator comprises the structure:

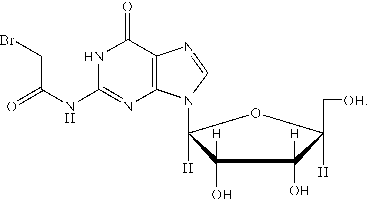

16. The othogonal GTPase modulator of claim 1, wherein the GTPase modulator comprises a substituted guanine ring having R1 attached at a C6 position, R2 attached at a C-7 position, and R3 attached at an N9 position;
wherein R1 is selected from the group consisting of a ketone, a thiol, a methyl thiol, an ethyl thiol, a propyl thiol, a butyl thiol, an O-thiol, an O-ethylthiol, an O-propylthiol, an O-butylthiol, an O-propyl group, an O-isopropyl group, an O-isobutyl group, an O-sec-butyl group an O-tert-butyl group, an O-(2,2-dimethyl)propyl group, an O-cyclohexyl group, an O-methylcyclohexyl group, an O-(2-cyclohexyl)ethyl group, an O-(3-cyclohexyl)propyl group, an O-phenyl group, an O-benzyl group, an O a (2-phenyl)ethyl group, an O-[2-(1-naphthyl)]ethyl group, an O-[2-(2-naphthyl)]ethyl group, an N-propyl group, an N-isopropyl group, an N-isobutyl group, an N-sec-butyl group, an N-tert-butyl group, an N-(2,2-dimethyl)propyl group, an N-cyclohexyl group, an N-methylcyclohexyl group, an N-(2-cyclohexyl)ethyl group, an N-(3-cyclohexyl)propyl group, an N-phenyl group, an N-benzyl group, an N-(2-phenyl)ethyl group, an N-[2-(1-naphthyl)]ethyl group, an N-[2-(2-naphthyl)]ethyl group, an O-[(3-maleimido)propylamido]ethyl group, an O-[(3-methyl)maleimido]ethyl group, an O-[(3,4-dimethyl)maleimido]ethyl group, an O-[(3,4-dimethyl)maleimido]propyl group, an-(2-N-acrylamido)ethyl group, an O-(n-N-acrylamido)alkyl group, an alkyl halide group; a (2-phenyl)ethyl group, a [2-(1-naphthyl)ethyl group, and a [2-(2-naphthyl)ethyl group
wherein R2 is selected from the group consisting of a ketone, a thiol, a methyl thiol, an ethyl thiol, a propyl thiol, a butyl thiol, an n-propyl group, an isopropyl group, an isobutyl group, a sec-butyl group, a tert-butyl group, a (2,2-dimethyl)propyl group, a cyclohexyl group, a methylcyclohexyl group, a (2-cyclohexyl)ethyl group, a (3-cyclohexyl)propyl group, a phenyl group, a benzyl group, a (2-phenyl)ethyl group, a pyridine group, a 3-pyrroline group, a [2-(1-naphthyl)]ethyl group, and a [2-(2-naphthyl)]ethyl group; and
wherein R3 comprises a hydrogen, a ribose sugar, a monophosphorylated ribose, a diphosphorylated ribose, a triphosphorylated ribose, a ribose comprising one or more caged phosphate groups, a benzyl group or a methyl-tert-butyl group.

17. The othogonal GTPase modulator of claim 1, wherein the GTPase modulator comprises a substituted guanine ring having a first substituent attached at a C6 position, a second substituent attached at a C-7 position, and a third substituent attached at an N9 position, and wherein at least one of the substituents comprises an electrophilic moiety.

18. The othogonal GTPase modulator of claim 17, wherein the electrophilic moiety comprises a [(3-maleimido)propylamido]ethyl group, a [(3-methyl)maleimido]ethyl group, a [(3,4-dimethyl)maleimido]ethyl group, a [(3,4-dimethyl)maleimido]propyl group, a (2-N-acrylamido)ethyl group, a (n-N-acrylamido)alkyl group, a thiol group, an alkyl halide or an alkyl thiol group.

19. The othogonal GTPase modulator of claim 1, wherein the electrophilic moiety comprises an O-linked substituent or an N-linked substituent.

20. The othogonal GTPase modulator of claim 1, wherein the GTPase modulator is:

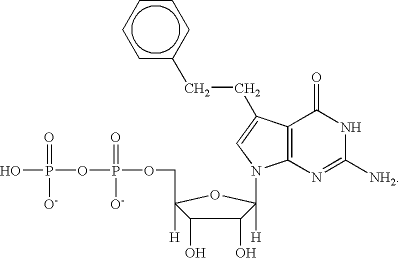

21. The othogonal GTPase modulator of claim 1, wherein the GTPase modulator is:

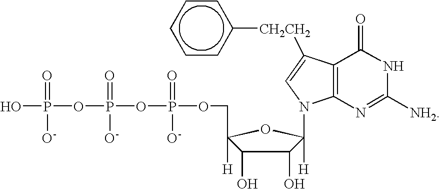

22. A mutant GTPase comprising a non-native amino acid at one or more amino acid positions that correspond to L19, F28, N116, K117 and T144 of H-Ras, which mutant GTPase binds to a GTPase modulator that modulates the mutant GTPase but does not substantially modulate a corresponding wild-type GTPase.

23. The mutant GTPase of claim 22, wherein the GTPase modulator comprises an inhibitor of GTPase activity.

24. The mutant GTPase of claim 22, wherein the GTPase modulator comprises an activator of GTPase activity.

25. The mutant GTPase of claim 22, wherein the non-native amino acid is selected from the group consisting of N116A, N116G and N116C.

26. The mutant GTPase of claim 22, wherein the GTPase modulator comprises a sulfhydryl group that forms a disulfide linkage with a sulfhydryl cysteine sidechain on the mutant GTPase.

27. The mutant GTPase of claim 26, wherein the mutant GTPase comprises N116C or T144C.

28. The mutant GTPase of claim 22, wherein the GTPase modulator comprises an electrophilic group that forms a covalent bond with an amino acid sidechain on the mutant GTPase.

29. The mutant GTPase of claim 28, wherein the mutant GTPase comprises N116C or T144C.

30. The mutant GTPase of claim 22, wherein the non-native amino acid is selected from the group consisting of L19A, L19G, and L19C.

31. The mutant GTPase of claim 22, wherein the mutant GTPase comprises a first non-native amino acid at a position that corresponds to N116 of H-Ras and a second non-native amino acid at a position that corresponds to L19 of H-Ras.

32. The mutant GTPase of claim 31, wherein the first non-native amino acid is N116A or N116G and the second non-native amino acid is L19A, L19G, or L19C.

33. The mutant GTPase of claim 32, wherein the first non-native amino acid is N116A or N116G and the second non-native amino acid is L19C.

34. The mutant GTPase of claim 22, wherein the GTPase is a Ras GTPase.

35. The mutant GTPase of claim 34, wherein the mutant GTPase comprises an amino acid sequence selected from the group consisting of SEQ ID NO: 2 to SEQ ID NO: 17.

36. The mutant GTPase of claim 35, wherein the mutant GTPase is encoded by a polynucleotide that comprises a nucleotide sequence selected from the group consisting of SEQ ID NO: 19 to SEQ ID NO: 34.

37. The mutant GTPase of claim 22, wherein the GTPase modulator comprises a structure selected from the group consisting of: a guanine ring modified at one or more of a C-6, N-7 and/or N-9 position; a guanosine triphosphate modified at a C-6 and/or N-7 position; a guanosine diphosphate modified at a C-6 and/or N-7 position; a guanosine monophosphate modified at a C-6 and/or N-7 position; and a guanosine modified at a C-2, C-6 and/or N-7 position.

38. The mutant GTPase of claim 22, wherein the GTPase modulator comprises a cell permeable compound.

39. The mutant GTPase of claim 22, wherein the GTPase modulator comprises an orthogonal GTPase modulator of claim 9

40. The mutant GTPase of claim 22, wherein the GTPase modulator comprises an orthogonal GTPase modulator of claim 10.

41. The mutant GTPase of claim 22, wherein the GTPase modulator comprises an orthogonal GTPase modulator of claim 11.

42. The mutant GTPase of claim 22, wherein the GTPase modulator comprises an orthogonal GTPase modulator of claim 12.

43. The mutant GTPase of claim 22, wherein the GTPase modulator comprises an orthogonal GTPase modulator of claim 13.

44. The mutant GTPase of claim 22, wherein the GTPase modulator comprises an orthogonal GTPase modulator of claim 14.

45. The mutant GTPase of claim 22, wherein the GTPase modulator comprises an orthogonal GTPase modulator of claim 15.

46. The mutant GTPase of claim 22, wherein the modulator is:

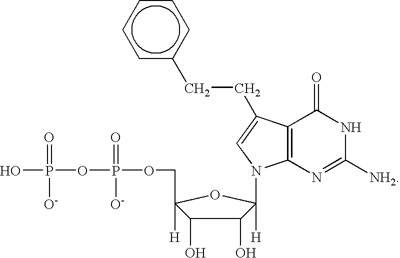

47. The mutant GTPase of claim 22, wherein the modulator is:

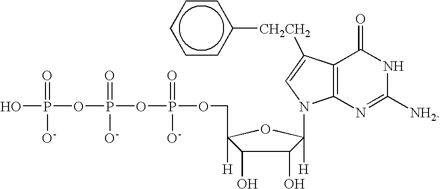

48. A complex comprising a mutant GTPase of claim 22 bound to a GTPase modulator that modulates the mutant GTPase but does not substantially modulate a corresponding wild-type GTPase.

49. The complex of claim 48, wherein the mutant GTPase comprises a double mutant having an alanine at first and second positions that correspond to L19 and N116 of H-Ras.

50. The complex of claim 48, wherein the GTPase modulator is:

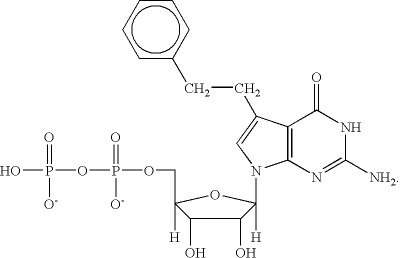

51. The complex of claim 48, wherein the GTPase modulator is

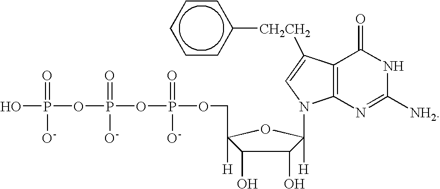

52. The complex of claim 48, wherein the GTPase modulator comprises:
a) a sulfhydryl group that forms a disulfide linkage with a sulfhydryl group on the mutant GTPase, or
b) an electrophilic group that forms a covalent bond with a sulfhydryl group on the mutant GTPase.

53. A host cell that comprises a mutant GTPase, which mutant GTPase comprises a non-native amino acid at one or more amino acid positions that correspond to N116, T144 and L19 of H-Ras, which mutant GTPase binds to a GTPase modulator that modulates the mutant GTPase but does not substantially modulate a corresponding wild-type GTPase.

54. The host cell of claim 53, wherein the host cell is selected from the group consisting of fibroblasts, myeloid leukemia cells, Raji cells, MIAPaCa-2 cells, PANC-1 cells, U-87 cells, U343 cells, U373 cells, EpH4 cells, human glioma cells, glioblastoma cells, and mammary epithelial cells.

55. The host cell of claim 53, wherein the host cells do not express a wild-type GTPase that corresponds to the mutant GTPase.

56. The host cell of claim 55, wherein a gene in the host cell that encodes the wild-type GTPase is disrupted.

57. The host cell of claim 53, wherein the host cell is present in an animal or a plant.

58. The host cell of claim 57, wherein the animal is a mammal.

59. A method of determining a GTPase function, the method comprising:
expressing at least one mutant GTPase in one or more host cells;
contacting the mutant GTPase with at least one GTPase modulator that binds to the mutant GTPase but does not substantially modulate a corresponding wild-type GTPase; and
detecting at least one result of applying the GTPase modulator to the cell, thereby determining the function of the GTPase.

60. The method of claim 59, wherein the mutant GTPase comprises a non-native amino acid at one or more amino acid positions that correspond to L19, N116 and T144 of H-Ras.

61. The method of claim 59, wherein the GTPase comprises one or more mutant Ras proteins.

62. The method of claim 59, wherein the host cells are selected from the group consisting of fibroblasts, myeloid leukemia cells, and Raji cells.

63. The method of claim 59, wherein the host cells do not express a wild-type GTPase that corresponds to the mutant GTPase.

64. The method of claim 63, wherein a gene that encodes the wild-type GTPase is disrupted in the host cell.

65. The method of claim 63, wherein the host cell is contacted with an antisense nucleic acid or an siRNA that inhibits expression of the corresponding wild-type GTPase but not the mutant GTPase.

66. The method of claim 59, wherein detecting comprises performing one or more assays to detect one or more functions of the mutant GTPase.

67. The method of claim 66, wherein the one or more assays comprise a GDP displacement assay.

68. The method of claim 59, wherein detecting further comprises determining one or more downstream response pathways affected by modulating the GTPase.

69. The method of claim 68, further comprising collecting data regarding the downstream response pathways and storing the data in at least one database.

70. The method of claim 59, wherein detecting comprises obtaining a gene expression profile of the cell in the presence and absence of the GTPase modulator to identify genes that are upregulated or downregulated in the presence of the GTPase modulator.

71. The method of claim 59, wherein contacting the mutant GTPase with the GTPase modulator comprises forming a covalent linkage between the GTPase modulator and an amino acid residue of the mutant GTPase, thereby modulating the activity of the GTPase.

72. The method of claim 71, wherein the mutant GTPase comprises a cysteine residue at one or more amino acid positions that correspond to L19, N116, or T144 of H-Ras; and
wherein the GTPase modulator comprises a sulfhydryl group or an electrophilic group.

73. The method of claim 59, wherein the GTPase modulator comprises at least one affinity label.

74. The method of claim 59, wherein the corresponding wild-type GTPase comprises Ras, and wherein the mutant GTPase has a decreased affinity for GTP and GDP.

75. The method of claim 59, wherein the GTPase modulator comprises a C-6 and N-7 substituted guanine moiety.

76. The method of claim 59, wherein contacting the mutant GTPase with the GTPase modulator further comprises forming a covalent linkage between the mutant GTPase and the GTPase modulator, leading to irreversible modulation of the GTPase.

77. The method of claim 59, wherein contacting the mutant GTPase with the GTPase modulator modulates binding of GTP or GDP to the mutant GTPase.

78. A method of modulating activity of a GTPase in a cell, the method comprising:
introducing into the cell a mutant GTPase that comprises a non-native amino acid at one or more amino acid positions that correspond to L19, N116 and T144 of H-Ras, which mutant GTPase binds to a GTPase modulator that modulates the mutant GTPase but does not substantially modulate a corresponding wild-type GTPase; and
contacting the mutant GTPase with the GTPase modulator, thereby competing with the wild-type GTPase for binding to one or more cellular effector molecule and reducing the activity of the GTPase in the cell.

79. The method of claim 78, wherein the cell is present in an animal.

80. The method of claim 78, further comprising:
administering an antisense nucleic acid or an siRNA that inhibits expression of the corresponding wild-type GTPase but not the mutant GTPase, thereby reducing or further reducing the GTPase activity in the cell.

81. The method of claim 78, wherein the GTPase modulator comprises a cell permeable compound, and wherein contacting the mutant GTPase with the GTPase modulator comprises providing a therapeutic composition of the GTPase modulator.

82. The method of claim 78, wherein the GTPase modulator comprises an inhibitor.

83. The method of claim 78, wherein the GTPase modulator comprises an activator.

84. A method of screening for proteins that specifically bind a GTPase, the method comprising:
a) providing a mutant GTPase, which mutant GTPase retains the effector specificity of a corresponding wild-type GTPase;
b) contacting the mutant GTPase with at least one orthogonal GTPase modulator, which GTPase modulator binds to the mutant GTPase but does not substantially inhibit or activate the corresponding wild-type GTPase, thereby providing a mutant GTPase-modulator complex;
c) contacting the mutant GTPase-modulator complex with at least one GTPase binding protein; and,
d) detecting the at least one GTPase binding protein.

85. The method of claim 84, wherein the binding protein comprises an effector of the GTPase.

86. The method of claim 84, comprising providing the mutant GTPase by providing a cell lysate comprising the mutant GTPase

87. The method of claim 84, comprising providing the mutant GTPase by expressing a polynucleotide sequence encoding the mutant GTPase in a cell.

88. The method of claim 84, wherein the mutant GTPase further comprises a sequence tag.

89. The method of claim 88, wherein the sequence tag comprises a GST- or a histidine-sequence tag.

90. The method of claim 88, wherein the mutant GTPase is bound to a solid substrate.

91. The method of claim 90, wherein the solid substrate comprises a bead.

92. The method of claim 84, wherein:
a) a dialyzed cell lysate comprising at least one orthogonal GTPase modulator is contacted with a mutant GTPase bound to a solid substrate, thereby providing a substrate-bound GTPase-modulator complex;
b) contacting the substrate-bound GTPase-modulator complex with at least one GTPase binding protein; and,
c) detecting the at least one GTPase binding protein.

93. The method of claim 92, further comprising:
eluting GTPase binding protein bound to the GTPase-modulator complex prior to detecting the at least one GTPase binding protein.

94. The method of claim 93, comprising eluting in a buffer comprising GTP.

95. The method of claim 93, comprising eluting in a buffer comprising GDP.

96. The method of claim 84, comprising detecting the at least one GTPase binding protein by two-dimensional electrophoresis.

97. The method of claim 84, further comprising identifying and/or isolating the at least one binding protein.

98. The method of claim 84, wherein the GTPase modulator is

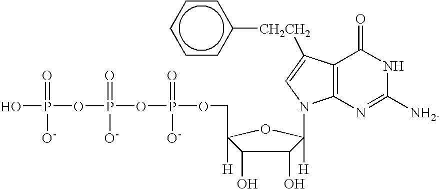

99. The method of claim 84, wherein the GTPase modulator is [chemical structure]
